(19) United States
(12) Patent Application Publication
(10) Pub. No.: US 2023/0218733 A1
(43) Pub. Date: Jul. 13, 2023

(54) RAPID VACCINE PLATFORM

(71) Applicant: CYTONUS THERAPEUTICS, INC., Columbus, OH (US)

(72) Inventors: Remo MOOMIAIE, Columbus, OH (US); Richard KLEMKE, Columbus, OH (US)

(21) Appl. No.: 18/190,838

(22) Filed: Mar. 27, 2023

Related U.S. Application Data
(63) Continuation of application No. 17/885,867, filed on Aug. 11, 2022.
Application No. 17/885,867 is a continuation of application No. PCT/US2021/017506, filed on Feb. 10, 2021.
(60) Provisional application No. 62/975,044, filed on Feb. 11, 2020.
(60) Provisional application No. 63/014,002, filed on Apr. 22, 2020.

(51) Int. Cl. A61K 39/00; C12N 5/0775; C12N 15/88; A61K 35/761; A61K 35/763; A61K 35/768; A61P 35/00

(52) U.S. Cl. CPC A61K 39/0011; C12N 5/0662; C12N 15/88; A61K 35/761; A61K 35/763; A61K 35/768; A61P 35/00; C12N 2770/20022; C12N 2770/20034; C12N 2770/20061; C12N 2770/20071; C12N 2710/16622; C12N 2710/16632; C12N 2710/16671; A61K 2039/5156; A61K 2039/5256

(57) ABSTRACT

Provided are methods of making and delivering vaccine compositions using an enucleated cell-based platform. Methods of clearing pathogenic infections in a subject using the enucleated cell-based platform is also provided. Such enucleated cell-based platform reduces the vaccine development timeline as compared with conventional biological vaccines, and improves vaccine efficacy.

### CROSS REFERENCE

This application is a continuation of U.S. patent application Ser. No. 17/885,867, filed on Aug. 11, 2022, which is a continuation of PCT Application No. PCT/US2021/017506 filed on Feb. 10, 2021, which claims the benefit of U.S. Provisional Application Ser. No. 62/975,044 filed on Feb. 11, 2020, and U.S. Provisional Application Ser. No. 63/014,002 filed on Apr. 2, 2020, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Oct. 5, 2022, is named 53712-706_302_SL.xml and is 1,091,372 bytes in size.

### BACKGROUND

The severe acute respiratory syndrome coronavirus 2 (SARS-CoV-2) pandemic and its attendant morbidity and mortality underscores a need for safe and efficacious vaccines that induce protective and durable immune responses. The pandemic also revealed severe shortcomings in the conventional vaccine development pipelines around the world to address urgent medical needs, such as the widespread transmission of Coronavirus disease 2019 (COVID-19). There exists an urgent and unmet need for a new vaccine development platform that can improve time-to-market for safe and efficacious vaccines and therapeutic agents to treat diseases or conditions caused by rapidly evolving pathogens, such as SARS-CoV-2.

### SUMMARY

Described herein, in some embodiments, is a cell without a nucleus, the cell without the nucleus comprising: one or more intracellular organelles for synthesis or secretion of a vaccine against a pathogen in absence of the nucleus. In some embodiments, the pathogen is a virus. In some embodiments, the virus is a coronavirus. In some embodiments, the coronavirus is a severe acute respiratory syndrome (SARS) coronavirus. In some embodiments, the SARS coronavirus is severe acute respiratory syndrome coronavirus 2 (SARS-CoV-2). In some embodiments, the virus is an oncolytic virus. In some embodiments, the pathogen is a bacterium. In some embodiments, the bacterium is Bacillus anthracis, Yersinia pestis, Francisella tularensis, Brucella, salmonella, Escherichia coli O157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof. In some embodiments, the pathogen is a toxin. In some embodiments, the toxin is Clostridium botulinumoxin, epsilon toxin of Clostridium perfringens, Staphylococcal enterotoxin B, or Ricin toxin from Ricinus communis, or any combination thereof. In some embodiments, the one or more intracellular organelles is an endoplasmic reticulum or a Golgi apparatus. In some embodiments, the vaccine is coupled to a surface of the cell without the nucleus. In some embodiments, the vaccine comprises a transmembrane domain that couples the vaccine to the surface of the cell without the nucleus. In some embodiments, the cell without the nucleus further comprises an immune-modulator comprising granulocyte-macrophage colony-stimulating factor. In some embodiments, the cell without the nucleus further comprises a homing receptor comprising: (a) Leukosialin; (b) L-selectin, lymphocyte function-associated antigen 1; (c) very late antigen-4; a portion of any one of (a) to (c); or any combination of (a) to (d). In some embodiments, the cell without the nucleus has a diameter that is between about 1 micrometers (μm) to 100 μm. In some embodiments, the diameter is about 8 μm. In some embodiments, the cell without the nucleus is viable following cryohibernation for at least 24 hours. In some embodiments, the cell without the nucleus is viable following cryohibernation for at least 48 hours. In some embodiments, the cell without the nucleus is viable following cryopreservation for at least 24 hours. In some embodiments, the cell without the nucleus is viable following lyophilization for at least 24 hours. In some embodiments, the cell without the nucleus is cryopreserved, cryohybernated, or lyophilized. In some embodiments, the cell without a nucleus is isolated or purified. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, the cell without the nucleus is not a red blood cell or a red blood cell precursor.

Described herein, in some embodiments, is a pharmaceutical formulation comprising: the cell without the nucleus or a plurality of the cell without the nucleus described herein; and a pharmaceutically acceptable: excipient, diluent, or carrier.

Described herein, in some embodiments, is a method of producing a vaccine, the method comprising: (a) removing a nucleus from a cell to produce an enucleated cell comprising one or more intracellular organelles for synthesis or secretion of a vaccine against a pathogen; and (b) introducing an exogenous mRNA encoding the vaccine to the enucleated cell, wherein the enucleated cell expresses the vaccine in absence of the nucleus. In some embodiments, the pathogen is a virus. In some embodiments, the virus is a coronavirus. In some embodiments, the coronavirus is a severe acute respiratory syndrome (SARS) coronavirus. In some embodiments, the SARS coronavirus is severe acute respiratory syndrome coronavirus 2 (SARS-CoV-2). In some embodiments, the virus is an oncolytic virus. In some embodiments, the pathogen is a bacterium. In some embodiments, the bacterium is Bacillus anthracis, Yersinia pestis, Francisella tularensis, Brucella, salmonella, Escherichia coli O157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof. In some embodiments, the pathogen is a toxin. In some embodiments, the toxin is Clostridium botulinum toxin, epsilon toxin of Clostridium perfringens, Staphylococcal enterotoxin B, or Ricin toxin from Ricinus communis, or any combination thereof. In some embodiments, the enucleated cell was stored at or below 4° C. to reversibly slow or stop biological activity of enucleated cell, and subsequently thawed prior to introducing in (b). In some embodiments, the cell without the nucleus was lyophilized and subsequently rehydrated prior to introducing in (b). In some embodiments, the enucleated cell was stored at or below −120° C. to reversibly slow or stop biological activity of enucleated cell, and subsequently thawed prior to introducing in (b). In some embodiments, the removing the nucleus from the cell in (a) is performed without differentiation of the cell. In some embodiments, the one or more intracellular organelles is an endoplasmic reticulum or a Golgi apparatus. In some embodiments, the cell without the nucleus has a diameter that is between about 1 micrometers (μm) to 100 μm. In some embodiments, the diameter is about 8 μm. In some embodiments, the method further comprises introducing to the cell prior to removing the nucleus in (a) an exogenous nucleic acid molecule with a nucleic acid sequence encoding an immune-modulator comprising granulocyte-macrophage colony-stimulating factor. In some embodiments, the method further comprises introducing to the cell prior to removing the nucleus in (a) an exogenous nucleic acid molecule with a nucleic acid sequence encoding a homing receptor comprising: Leukosialin; L-selectin, lymphocyte function-associated antigen 1; very late antigen-4; C-X-C chemokine receptor type 3; CD44 antigen; C-C chemokine receptor type 7; a portion of any one of the homing receptor thereof; or any combination of any one of the homing receptor thereof. In some embodiments, the method further comprises introducing to the cell without the nucleus an exogenous mRNA molecule comprising a sequence encoding an immune-modulator comprising granulocyte-macrophage colony-stimulating factor. In some embodiments, the method further comprises introducing to the cell without the nucleus an exogenous mRNA molecule comprising a sequence encoding a homing receptor comprising: Leukosialin; L-selectin, lymphocyte function-associated antigen 1; very late antigen-4; C-X-C chemokine receptor type 3; CD44 antigen; C-C chemokine receptor type 7; a portion of any one of the homing receptor thereof; or any combination of any one of the homing receptor thereof. In some embodiments, the cell without the nucleus is not a red blood cell or a red blood cell precursor.

Described herein, in some embodiments, is a method of delivering a vaccine against severe acute respiratory syndrome coronavirus 2 (SARS-CoV-2) to a subject, the method comprising: administering to the subject a cell without a nucleus comprising one or more intracellular organelles for synthesis or secretion of the vaccine against SARS-CoV-2 in absence of the nucleus. In some embodiments, the one or more intracellular organelles is an endoplasmic reticulum or a Golgi apparatus. In some embodiments, the cell without the nucleus further comprises an immune-modulator comprising granulocyte-macrophage colony-stimulating factor. In some embodiments, the cell without the nucleus further comprises a homing receptor comprising: Leukosialin; L-selectin, lymphocyte function-associated antigen 1; very late antigen-4; C-X-C chemokine receptor type 3; CD44 antigen; C-C chemokine receptor type 7; a portion of any one of the homing receptor thereof; or any combination of any one of the homing receptor thereof. In some embodiments, the cell without the nucleus has a diameter that is between about 1 micrometers (μm) to 100 μm. In some embodiments, the diameter is about 8 μm. In some embodiments, administrating comprises systemic administration. In some embodiments, the cell without the nucleus is administered in a dosage amount of between about 10³ cells/kg body weight to about 10¹² cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years. In some embodiments, the subject is human. In some embodiments, the method further comprises administering an adjuvant. In some embodiments, the cell without the nucleus is not a red blood cell or a red blood cell precursor.

Described herein, in some embodiments, is a kit comprising: a plurality of cells substantially free of nuclei, wherein at least one cell without a nucleus of the plurality comprises one or more intracellular organelles for synthesis or secretion of a vaccine against a pathogen in absence of the nucleus; and instructions for administering the plurality of cells substantially free of nuclei to a subject. In some embodiments, the plurality of cells substantially free of nuclei are cryopreserved, cryo-hibernated, or lyophilized. In some embodiments, the kit further comprises instructions for restoring biological activity of the plurality of cells substantially free of nuclei prior to administering the plurality of cells substantially free of nuclei to the subject. In some embodiments, the kit further comprises instructions for introducing an exogenous mRNA encoding the vaccine to the enucleated cell.

Described herein, in some embodiments, is a cell without a nucleus, the cell without the nucleus comprising: one or more intracellular organelles for synthesis of a receptor for a pathogen antigen or a pathogen antigen-binding fragment thereof in absence of the nucleus, wherein the receptor or an expression level of the receptor is exogenous to the cell without the nucleus. In some embodiments, the one or more intracellular organelles is an endoplasmic reticulum or a Golgi apparatus. In some embodiments, the receptor for the pathogen antigen or the pathogen antigen-binding fragment thereof is coupled to a surface of the cell without the nucleus. In some embodiments, the receptor for the pathogen antigen or the pathogen antigen-binding fragment thereof comprises a transmembrane domain within a cell membrane of the cell without the nucleus. In some embodiments, the cell without the nucleus further comprises an exogenous mRNA molecule having a sequence encoding an immune-modulator comprising granulocyte-macrophage colony-stimulating factor, or a portion thereof. In some embodiments, the cell without the nucleus has a diameter that is between about 1 micrometers (μm) to 100 μm. In some embodiments, the diameter is about 8 μm. In some embodiments, the cell without the nucleus is viable following cryohibernation for at least 24 hours. In some embodiments, the cell without the nucleus is viable following cryohibernation for at least 48 hours. In some embodiments, the cell without the nucleus is viable following cryopreservation for at least 24 hours. In some embodiments, the cell without the nucleus is viable following lyophilization for at least 24 hours. In some embodiments, the cell without the nucleus is cryopreserved, cryohybernated, or lyophilized. In some embodiments, the cell without a nucleus is isolated or purified. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, the cell without a nucleus is isolated or purified. In some embodiments, the cell further comprises a neutralizing antibody that blocks binding between the pathogen antigen and its natural receptor produced by a host cell. In some embodiments, the neutralizing antibody is synthesized by the one or more intracellular organelles of the cell without the nucleus. In some embodiments, the cell further comprises: a homing receptor comprising: Leukosialin; L-selectin, lymphocyte function-associated antigen 1; very late antigen-4; C-X-C chemokine receptor type 3; CD44 antigen; C-C chemokine receptor type 7; a portion of any one of the homing receptor thereof; or any combination of any one of the homing receptor thereof. In some embodiments, the pathogen is a virus. In some embodiments, the virus is a coronavirus. In some embodiments, the coronavirus is a severe acute respiratory syndrome (SARS) coronavirus. In some embodiments, the SARS coronavirus is severe acute respiratory syndrome coronavirus 2 (SARS-CoV-2). In some embodiments, the virus is an oncolytic virus. In some embodiments, the pathogen is a bacterium. In some embodiments, the bacterium is Bacillus anthracis, Yersinia pestis, Francisella tularensis, Brucella, salmonella, Escherichia coli O157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof. In some embodiments, the pathogen is a toxin. In some embodiments, the toxin is Clostridium botulinum toxin, epsilon toxin of Clostridium perfringens, Staphylococcal enterotoxin B, or Ricin toxin from Ricinus communis, or any combination thereof. In some embodiments, the vaccine is a vaccine described herein. In some embodiments, the cell without the nucleus is not a red blood cell or a red blood cell precursor.

Described herein, in some embodiments, is a method of reducing an infection by a pathogen in a subject or a method of reducing a pathogen in the process of infecting a subject, the method comprising: administering to a subject the cell without the nucleus described herein or the pharmaceutical formulation described herein, thereby trapping a pathogen having the pathogen antigen in the cell and preventing the pathogen from propagating within the cell. In some embodiments, the pathogen is cleared from the subject in fewer than or equal to about 14 days following administration. In some embodiments, the cell without the nucleus releases a neutralizing antibody or nanobody, thereby blocking binding between the pathogen antigen of the pathogen and its natural receptor produced by a host cell. In some embodiments, the administrating comprises systemic administration. In some embodiments, the cell without the nucleus is administered in a dosage amount of between about 10³ cells/kg body weight to about 10¹² cells/kg body weight. In some embodiments, the cell without the nucleus is administered to the subject twice within at least an hour, 2 hours, 4 hours, 6 hours, 8 hours, 12 hours, 1 day, 2 days, a week, 2 weeks, 3 weeks, a month, 2 months, 3 months, 4 months, 5 months, 6 months, 7 months, 8 months, 9 months, 10 months, 11 months, a year, 2 years, 3 years, or 4 years. In some embodiments, the pathogen is a virus. In some embodiments, the virus is a coronavirus. In some embodiments, the coronavirus is a severe acute respiratory syndrome (SARS) coronavirus. In some embodiments, the SARS coronavirus is severe acute respiratory syndrome coronavirus 2 (SARS-CoV-2). In some embodiments, the virus is an oncolytic virus. In some embodiments, the pathogen is a bacterium. In some embodiments, the bacterium is Bacillus anthracis, Yersinia pestis, Francisella tularensis, Brucella, salmonella, Escherichia coli O 157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof. In some embodiments, the pathogen is a toxin. In some embodiments, the toxin is Clostridium botulinum toxin, epsilon toxin of Clostridium perfringens, Staphylococcal enterotoxin B, or Ricin toxin from Ricinus communis, or any combination thereof. In some embodiments, the vaccine is a vaccine described herein. In some embodiments, the cell without the nucleus further comprises an immune-modulator comprising granulocyte-macrophage colony-stimulating factor. In some embodiments, the cell without the nucleus further comprises a homing receptor that is specific to a ligand expressed on one or more cells in lymph tissue. In some embodiments, the homing receptor comprises C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the cell without the nucleus has a diameter that is between about 1 micrometers (μm) to 100 μm. In some embodiments, the cell without the nucleus has a diameter that is about 8 μm. In some embodiments, the cell without the nucleus is viable following cryohibernation for at least 24 hours. In some embodiments, the cell without the nucleus is viable following cryohibernation for at least 48 hours. In some embodiments, the cell without the nucleus is viable following cryopreservation for at least 24 hours. In some embodiments, the cell without the nucleus is viable following lyophilization for at least 24 hours. In some embodiments, the cell without the nucleus is cryopreserved, cryohybernated, or lyophilized. In some embodiments, the cell without a nucleus is isolated or purified. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, the cell without the nucleus is not a red blood cell or a red blood cell precursor.

Aspects disclosed herein provide a cell without a nucleus, the cell comprising: one or more intracellular organelles for synthesis or secretion, in absence of the nucleus, of a vaccine against a virus encoded by a sequence with a sequence identity that is greater than or equal to about 80%, 81%, 82%, 83%, 84%, 85%, 86%, 87% 88%, 89% 90%, 91%, 92%, 93%, 94%, 95%, 96%, 97%, 98%, 99% to one or more of SEQ ID NOs: 1, 301-347, or 501-512. In some embodiments, the cell without the nucleus is not a red blood cell or a red blood cell precursor. In some embodiments, the cell without the nucleus is derived from a nucleated parent cell to which the one or more intracellular organelles is endogenous. In some embodiments, the virus is a coronavirus. In some embodiments, the vaccine composition is a DNA, a RNA, an antigenic peptide, an attenuated live virus, or an inactivated virus, or a combination thereof. In some embodiments, the antigenic peptide comprises an amino acid sequence having a sequence identity that is greater than or equal to about 80%, 81%, 82%, 83%, 84%, 85%, 86%, 87% 88%, 89% 90%, 91%, 92%, 93%, 94%, 95%, 96%, 97%, 98%, 99% to one or more of SEQ ID NOs: 2, 3-7, 151-154, 251-260, 401-447, 551-562, 651-660, 751-761, 851-859, 951-984, 1051-1057, or 1151-1153. In some embodiments, the antigenic peptide comprises an amino acid sequence having a sequence identity that is greater than or equal to about 80%, 81%, 82%, 83%, 84%, 85%, 86%, 87% 88%, 89% 90%, 91%, 92%, 93%, 94%, 95%, 96%, 97%, 98%, 99% to one or more of SEQ ID NOs: 2, 8, 401-447 or 551-562. In some embodiments, the antigenic peptide is encoded from a nucleic acid sequence having a sequence identity that is greater than or equal to about 80%,81%, 82%, 83%, 84%, 85%, 86%, 87% 88%, 89% 90%, 91%, 92%, 93%, 94%, 95%, 96%, 97%, 98%, 99% to one or more of SEQ ID NOs: 101-104, 201-209, 301-347, 501-512, 601-610, 701-711, 801-809, 901-934, 1001-1007, or 1101-1103. In some embodiments, the antigenic peptide further comprises an amino acid sequence encoding albumin, or a portion thereof. In some embodiments, the vaccine is coupled to a surface of the cell. In some embodiments, the vaccine is secretory. In some embodiments, the cell without the nucleus further comprises an immune-modulator comprising granulocyte-macrophage colony-stimulating factor. In some embodiments, the cell without the nucleus further comprises a homing receptor that is specific to a ligand expressed on one or more cells in lymph tissue. In some embodiments, the homing receptor comprises C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the cell without the nucleus has a diameter that is between about 1 micrometers (μm) to 100 μm. In some embodiments, the cell without the nucleus has a diameter that is about 8 μm. In some embodiments, the cell without the nucleus is viable following cryohibernation for at least 24 hours. In some embodiments, the cell without the nucleus is viable following cryopreservation for at least 24 hours. In some embodiments, the cell without the nucleus is viable following cryohibernation for at least 48 hours. In some embodiments, the cell without the nucleus is viable following cryopreservation for at least 48 hours. In some embodiments, the cell without the nucleus is viable following lyophilization for at least 24 hours. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, the cell without the nucleus is cryopreserved, cryohybernated, or lyophilized. In some embodiments, synthesis or secretion of the vaccine in the absence of the nucleus is performed by the cell without the nucleus for greater than or equal to about 3 days. In some embodiments, the cell without the nucleus is in a pharmaceutically acceptable carrier. In some embodiments, the cell without the nucleus is in a dosage of between about 10³ cells/kg body weight to about 10¹² cells/kg body weight. In some embodiments, the cell without the nucleus is in a dosage of between at least or about 10³, 10⁴, 10⁵, 10⁶, 10⁷, 10⁸, 10⁹, 10¹⁰,10¹¹, 10¹²cells/kg body. In some embodiments, the cell without the nucleus is in a dosage of between at most or about 10³, 10⁴, 10⁵, 10⁶, 10⁷, 10⁸, 10⁹, 10¹⁰, 10¹¹, 10¹² cells/kg body. In some embodiments, the cell without the nucleus is isolated and purified.

Aspects disclosed herein provide a cell without a nucleus, the cell comprising: one or more intracellular organelles for synthesis or secretion of a vaccine against a bacteria or a toxin in absence of the nucleus. In some embodiments, the cell without the nucleus is not a red blood cell or a red blood cell precursor. In some embodiments, the cell without the nucleus is derived from a nucleated parent cell to which the one or more intracellular organelles is endogenous. In some embodiments, the toxin is Clostridium botulinum toxin, epsilon toxin of Clostridium perfringens, Staphylococcal enterotoxin B, or Ricin toxin from Ricinus communis, or any combination thereof. In some embodiments, the bacterium is Bacillus anthracis, Yersinia pestis, Francisella tularensis, Brucella, salmonella, Escherichia coli O 157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof. In some embodiments, the vaccine is coupled to a surface of the cell. In some embodiments, the vaccine is secretory. In some embodiments, the cell without the nucleus further comprises an immune-modulator comprising granulocyte-macrophage colony-stimulating factor. In some embodiments, the cell without the nucleus further comprises a homing receptor that is specific to a ligand expressed on one or more cells in lymph tissue. In some embodiments, the homing receptor comprises C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the cell without the nucleus has a diameter that is between about 1 micrometers (μm) to 100 μm. In some embodiments, the cell without the nucleus has a diameter that is about 8 μm. In some embodiments, the cell without the nucleus is viable following cryohibernation for at least 24 hours. In some embodiments, the cell without the nucleus is viable following cryopreservation for at least 24 hours. In some embodiments, the cell without the nucleus is viable following cryohibernation for at least 48 hours. In some embodiments, the cell without the nucleus is viable following cryopreservation for at least 48 hours. In some embodiments, the cell without the nucleus is viable following lyophilization for at least 24 hours. In some embodiments, the cell without the nucleus is cryopreserved, cryohybernated, or lyophilized. In some embodiments, the cell without a nucleus is isolated or purified. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, the cell without the nucleus is cryopreserved, cryohybernated, or lyophilized. In some embodiments, synthesis or secretion of the vaccine in the absence of the nucleus is performed by the cell without the nucleus for greater than or equal to about 3 days. In some embodiments, the cell without the nucleus is in a pharmaceutically acceptable carrier. In some embodiments, the cell without the nucleus is in a dosage of between about 10³ cells/kg body weight to about 10¹² cells/kg body weight. In some embodiments, the cell without the nucleus is in a dosage of between at least or about 10³, 10⁴, 10⁵, 10⁶, 10⁷, 10⁸, 10⁹, 10¹⁰, 10¹¹, 10¹² cells/kg body. In some embodiments, the cell without the nucleus is in a dosage of between at most or about 10³, 10⁴, 10⁵, 10⁶, 10⁷, 10⁸, 10⁹, 10¹⁰, 10¹¹, 10¹² cells/kg body. In some embodiments, the cell without the nucleus is isolated and purified.

Aspects disclosed here provide a population of cells comprising a plurality of the cell without the nucleus described herein.

Aspects disclosed herein provide methods of delivering to a subject a vaccine, the method comprising administering to the subject a first dose of a cell of the plurality of cells described herein. In some embodiments, the subject becomes vaccinated following administration. In some embodiments, administering is performed at least 24 hours following removing the cell from cryohibernation or cryopreservation. In some embodiments, administering is performed at least 48 hours following removing the cell of from cryohibernation or cryopreservation. In some embodiments, the cell without the nucleus is viable following lyophilization for at least 24 hours. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, the cell synthesizes or secretes the vaccine in the subject in the absence of the nucleus for greater than or equal to about 3 days. In some embodiments, the cell synthesizes or secretes the vaccine in the subject in the absence of the nucleus for between about 3 to 5 days. In some embodiments, methods further comprise administering a second dose of a second cell of the population of cells to the subject at least 1 month following administering the first dose of the cell. In some embodiments, methods further comprise administering a third dose of a second cell of the population of cells to the subject at least 2 months following administering the first dose of the cell.

Aspects disclosed herein provide methods comprising administering to a subject in need thereof a cell without a nucleus that synthesizes or secretes a therapeutic agent in an absence of the nucleus, wherein the therapeutic agent is therapeutically effective to treat a disease or condition associated with an infection by a virus encoded by a sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% of SEQ ID NO: 1. In some embodiments, methods further comprise treating the disease or condition in the subject. In some embodiments, the therapeutic agent is: (a) an agonist of interleukin 10; (b) an antagonist of interleukin 10; (c) interleukin 6; (d) tumor necrosis factor (TNF); (e) a portion of any one of (a) to (d); or (e) a combination of any of (a) to (d). In some embodiments, the agonist of interleukin 10 is interleukin 10, or portion thereof, comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 13. In some embodiments, the agonist of interleukin 10, or portion thereof, further comprises an amino acid sequence encoding albumin or a portion thereof. In some embodiments, the therapeutic agent is secreted by the cell. In some embodiments, the agonist of interleukin 6, or portion thereof, comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 14. In some embodiments, the agonist of TNF comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 15. In some embodiments, the cell without the nucleus further comprises a homing receptor that is specific to a ligand expressed on one or more cells in lung tissue of the subject. In some embodiments, the homing receptor comprises P-selectin glycoprotein ligand-1, C-C Motif Chemokine Receptor 2, or C-X-C Motif Chemokine Receptor 4, or a combination thereof. In some embodiments, the cell further comprises a homing receptor that is specific to a ligand expressed on one or more cells in lymph tissue of the subject. In some embodiments, the homing receptor comprises C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the cell without the nucleus further comprises an immune-modulator comprising granulocyte-macrophage colony-stimulating factor (GM-CSF). In some embodiments, the disease or condition is a respiratory disease or condition. In some embodiments, the disease or condition comprises symptoms of coronavirus disease (COVID). In some embodiments, the COVID is COVID-19.

Aspects disclosed herein provide methods comprising administering to a subject in need thereof a cell without a nucleus that synthesizes or secretes a therapeutic agent in an absence of the nucleus, wherein the therapeutic agent is therapeutically effective to treat a disease or condition caused, at least in part, by an infection by a pathogen. In some embodiments, the pathogen is a virus, a bacterium, a fungus, or a toxin. In some embodiments, the virus is an oncolytic virus. In some embodiments, the toxin is Clostridium botulinum toxin, epsilon toxin of Clostridium perfringens, Staphylococcal enterotoxin B, or Ricin toxin from Ricinus communis, or any combination thereof. In some embodiments, the bacterium is Bacillus anthracis, Yersinia pestis, Francisella tularensis, Brucella, salmonella, Escherichia coli O 157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof. In some embodiments, the therapeutic agent is: (a) an agonist of interleukin 10; (b) an antagonist of interleukin 10 (e.g., GIT27, AS101, mesopram, or rituximab); (c) interleukin 6; (d) tumor necrosis factor (TNF); (e) a portion of any one of (a) to (d); or (e) a combination of any of (a) to (d). In some embodiments, the agonist of interleukin 10 is interleukin 10, or portion thereof, comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 13. In some embodiments, the agonist of interleukin 10, or portion thereof, further comprises an amino acid sequence encoding albumin or a portion thereof. In some embodiments, the therapeutic agent is secreted by the cell. In some embodiments, the agonist of interleukin 6, or portion thereof, comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 14. In some embodiments, the agonist of TNF comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 15. In some embodiments, the cell without the nucleus further comprises a homing receptor that is specific to a ligand expressed on one or more cells in lung tissue of the subject. In some embodiments, the homing receptor comprises P-selectin glycoprotein ligand-1, C-C Motif Chemokine Receptor 2, or C-X-C Motif Chemokine Receptor 4, or a combination thereof. In some embodiments, the cell further comprises a homing receptor that is specific to a ligand expressed on one or more cells in lymph tissue of the subject. In some embodiments, the homing receptor comprises C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the cell without the nucleus further comprises an immune-modulator comprising granulocyte-macrophage colony-stimulating factor (GM-CSF). In some embodiments, the disease or condition is provided in Tables 3-6.

Aspects disclosed herein provide methods of treating a pathogen-associated disease or condition, the method comprising: (a) administering to a subject with an infection by a pathogen a plurality of cells substantially free of nuclei, thereby sequestering the pathogen from the subject in vivo by (i) permitting infection of at least one cell without a nucleus of the plurality of cells administered to the subject in (a) by the pathogen; and (ii) following (i), preventing propagation of the pathogen within the at least one cell without the nucleus; and (b) treating the pathogen-associated disease or condition by at least one of: (i) removing or reducing the pathogen from the at least one cell of the plurality of cells in vivo; and (ii) substantially removing the at least one cell without the nucleus from the subject. In some embodiments, the at least one cell without the nucleus comprises a homing receptor that is specific to a ligand expressed on one or more cells in lymph tissue of the subject. In some embodiments, the homing receptor comprises C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the pathogen is a coronavirus. In some embodiments, the coronavirus is encoded by a nucleic acid sequence with a sequence identity that is greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NO: 1. In some embodiments, the at least one cell without the nucleus comprises an immune-modulator comprising: (a) granulocyte-macrophage colony-stimulating factor; (b) a cytokine; (c) a portion of (a) or (b); or (d) any combination of (a) to (c). In some embodiments, the at least one cell without the nucleus comprises one or more intracellular organelles sufficient to synthesize or secrete one or more of (a) to (d). In some embodiments, the cytokine comprises an amino acid sequence with a sequence identity of greater than or equal to about 80%, 81%, 82%, 83%, 84%, 85%, 86%, 87% 88%, 89% 90%, 91%, 92%, 93%, 94%, 95%, 96%, 97%, 98%, 99% to SEQ ID NOs: 13, 14, or 15, or a combination thereof. In some embodiments, the cytokine is secretory. In some embodiments, the at least one cell without the nucleus has a diameter that is between bout 1 micrometers (μm) to 100 μm. In some embodiments, the at least one cell without the nucleus has a diameter is about 8 μm. In some embodiments, methods further comprise removing the plurality of cells substantially free of nuclei from cryohybernation or cryopreservation prior to administering in (a). In some embodiments, the plurality of cells substantially free of nucleic is viable for at least 24 hours following removing the plurality of cells substantially free of nuclei from cryohybernation, cryopreservation, or lyophilization. In some embodiments, the cell without the nucleus is viable following lyophilization for at least 24 hours. In some embodiments, the cell without the nucleus is cryopreserved, cryohybernated, or lyophilized. In some embodiments, the cell without a nucleus is isolated or purified. In some embodiments, viability is measured using Trypan blue dye exclusion as described herein. In some embodiments, the Trypan blue dye exclusion is performed by: (a) centrifuging an aliquot of a plurality of the cell without the nucleus in a suspension to create a cell pellet; (b) resuspending the cell pellet in serum-free medium to produce a serum-free cell suspension; (c) mixing 1 part Trypan blue dye and 1 part of the serum-free cell suspension; (d) counting the plurality of the cells without the nucleus within 3-5 minutes of (c), wherein at least some of the plurality of cells without the nucleus are unstained with the Trypan blue dye, which is indicative of viability. In some embodiments, viability is measured using Annexin-5 cell surface staining as described herein. In some embodiments, treating the pathogen-associated disease or condition in (b) is by removing or reducing the pathogen from the at least one cell of the plurality of cells. In some embodiments, the at least one cell comprises an anti-viral agent effective to reduce or removing the pathogen from the at least one cell. In some embodiments, treating the pathogen-associated disease or condition in (b) is by substantially removing the at least one cell without the nucleus from the subject. In some embodiments, the plurality of cells are not red blood cells or red blood cell precursors. In some embodiments, the at least one cell without the nucleus comprises an heterologous polynucleotide encoding a neutralizing antibody that blocks binding between the pathogen and a pathogen-recognized receptor expressed by a cell of the subject. In some embodiments, methods further comprise secreting the neutralizing antibody, by the at least one cell without the nucleus, in the absence of the nucleus, thereby reducing or ameliorating binding between the pathogen and a pathogen-recognized moiety of a cell of the subject. In some embodiments, the pathogen is a virus, bacterium, toxin, or fungus. In some embodiments, the virus is an oncolytic virus. In some embodiments, the virus is a coronavirus. In some embodiments, the coronavirus is SARS-CoV-2, or a variant thereof. In some embodiments, the toxin is Clostridium botulinum toxin, epsilon toxin of Clostridium perfringens, Staphylococcal enterotoxin B, or Ricin toxin from Ricinus communis, or any combination thereof. In some embodiments, the bacterium is Bacillus anthracis, Yersinia pestis, Francisella tularensis, Brucella, salmonella, Escherichia coli O 157:H7, Shigella, Burkholderia mallei, Burkholderia pseudomallei, Chlamydia psittaci, Coxiella burnetii, Rickettsia prowazekii, Vibrio cholerae, or Cryptosporidium parvum, or any combination thereof.

### Incorporation by Reference

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Some novel features of the methods and compositions disclosed herein are set forth in the present disclosure. A better understanding of the features and advantages of the methods and compositions disclosed herein will be obtained by reference to the following detailed description that sets forth illustrative embodiments, in which the principles of the disclosed compositions and methods are utilized, and the accompanying drawings of which:

FIG. 1 shows a process for engineering cells for the rapid virus vaccine platform according to an embodiment of the present disclosure.

FIG. 2 shows a timeline for production of a vaccine using the rapid virus vaccine platform according to an embodiment of the present disclosure, as compared to a traditional vaccine development timeline.

FIG. 3 shows a process for deploying the rapid virus vaccine platform to address a newly identified virus according to an embodiment of the present disclosure.

FIG. 4 shows a process by which cytoplasts described herein trap and clear live virus (e.g., coronavirus) according to an embodiment of the present disclosure.

FIG. 5 shows non-limiting examples of the benefits of the rapid virus vaccine platform described herein.

FIG. 6A is a representative line graph showing the viability of MSC and MSC-derived cytoplasts immediately after recovery from cryohibernation at 4 degrees Celsius for the indicated amounts of time. Viability was assessed in an automated cell count (Cell Countess) using Trypan blue dye exclusion and displayed as a ratio to the number of input cells.

FIG. 6B is a representative bar graph comparing the migrated MSC and MSC-derived cytoplasts in a Boyden chamber assay immediately after recovery from cryohibernation at 4 degrees Celsius for the indicated amounts of time. Cells and cytoplasts were allowed to migrate for 3 hours with either no serum (negative control) or 10% premium FBS (P-FBS) as a chemoattractant in the bottom chamber, and counts were normalized to loading controls.

FIG. 7A is a schematic representation of an interleukin 10 (IL-10) mRNA transfected into MSC and cytoplasts. Kozak sequence was added in front of the start codon of the IL-10 mRNA coding region (CDS). 5′UTR and 3′UTR of human beta globin (HBB) mRNA were added respectively to the 5′ and 3′ end of IL-10 CDS. An artificial 5′Cap was added to the 5′ end of the IL-10 mRNA and the pseudouridine modification was engineered to increase mRNA stability.

FIG. 7B is a bar graph showing IL-10 concentration in the culture medium of transfected (++) or non-transfected (−−) MSC or MSC-derived cytoplasts. MSC-derived cytoplasts were transfected with IL-10 mRNA, then seeded in a 24 well plate at 2.5×10⁴ cells/well. Conditioned medium (CM) was collected 24 hours after transfection and the IL-10 concentration determined by ELISA.

FIG. 7C is an immunoblot showing protein expression of Stat3 and phosphorylated Stat3 (P-Stat3, a marker of IL-10 activation) in serum-starved RAW macrophage cells treated with the indicated conditioned media (CM) from MSCs or cytoplasts treated as in FIG. 7B for 1 hour. Untreated=no CM treated control. Complete medium=RAW cells treated with MSC complete culture medium. MSC Ctrl=RAW cells treated with CM from non-transfected MSCs. MSC IL-10=RAW cells treated with CM from IL-10 mRNA transfected MSCs. Cytoplast Ctrl=RAW cells treated with CM from non-transfected cytoplasts. Cytoplasts IL-10=RAW cells treated with CM from IL-10 mRNA transfected cytoplasts.

FIG. 7D is a bar graph showing the concentration of secreted IL-10 cytokine in the mouse blood as determined by ELISA. MSC or MSC-derived cytoplasts were treated as in FIG. 7B and retro-orbitally injected into the vasculature of C57BL/6 mice. Two hours after injection, animals were euthanized, and blood samples were collected by cardiac puncture. Mean±SEM; n=3.

FIG. 8A are representative bright field microscopy images of Crystal Violet-stained MSCs or MSC-derived cytoplasts in a Boyden chamber assay that invaded to the undersurface of 8.0 μm porous filters coated with Basement Membrane Extract (BME) towards 10% FBS as a chemoattractant for 24 hours. Negative=no FBS (negative control). Scale Bar=50 μm.

FIG. 8B is a representative bar graph showing the ratio of MSC or MSC-derived cytoplasts treated as in FIG. 8A that invaded to the undersurface of the membrane compared to the loading control. Mean±SEM; n=18.

FIG. 9A is representative epifluorescence microscopy images (upper panel) and phase contrast microscopy images (lower panel) of MSCs and cytoplasts in suspension media. Actin cortex was stained with Lifeact RFP, while the cell nucleus was stained with Vybrant® Dyecycle™ Green. Arrows point to cytoplasts and arrowhead points to MSC nucleus. Scale bar=20 μm.

FIG. 9B is a representative scatter plot showing the size distribution of MSCs and cytoplasts as measured with Nikon Element software. Mean±SEM; n=80.

FIG. 9C is a representative bar graph showing the detected Vybrant® DiD-labeled MSCs or cytoplasts present in lung. MSCs or cytoplasts were labeled with DiD dye and retro-orbitally injected into the vasculature of C57BL/6 mice. Tissues were harvested after 24 hours and cell suspensions analyzed by flow cytometry. Mean±SEM; n=3.

FIG. 9D is a representative bar graph showing the detected Vybrant® DiD labeled MSCs or cytoplasts present in liver. Mean±SEM; n=3. MSCs or cytoplasts were labeled with DiD dye and retro-orbitally injected into the vasculature of C57BL/6 mice. Tissues were harvested after 24 hours and cell suspensions analyzed by flow cytometry.

FIG. 10A is a representative scatter plot showing the number of DiD-labeled MSCs or cytoplasts detected in the lung. MSCs were cultured under standard adherent conditions (2D) or in suspension by the handing drop method (3D) to generate 3D cytoplasts. MSCs and cytoplasts were labeled with Vybrant® DiD dye and retro-orbitally injected into the vasculature of C57BL/6 mice. Tissues were harvested after 24 hours and cell suspensions analyzed by flow cytometry. Mean±SEM; n=2.

FIG. 10B is a representative scatter plot showing the number of DiD-labeled MSCs or cytoplasts detected in the liver. MSCs were cultured under standard adherent conditions (2D) or in suspension by the handing drop method (3D) to generate 3D cytoplasts. MSCs and cytoplasts were labeled with Vybrant® DiD dye and retro-orbitally injected into the vasculature of C57BL/6 mice. Tissues were harvested after 24 hours and cell suspensions analyzed by flow cytometry. Mean±SEM; n=2.

FIG. 10C is a representative scatter plot showing the number of Vybrant® DiD-labeled MSCs or cytoplasts detected in the spleen. MSCs were cultured under standard adherent conditions (2D) or in suspension by the handing drop method (3D) to generate 3D cytoplasts. MSCs and cytoplasts were labeled with DiD dye and retro-orbitally injected into the vasculature of C57BL/6 mice. Tissues were harvested after 24 hours and cell suspensions analyzed by flow cytometry. Mean±SEM; n=2.

FIG. 11A-11B illustrate Epifluorescent microscopy images of nucleated parental MSCs (top) and MSC-derived cytoplast (bottom) infected with VSV-GFP (arrows) at MOI 0.05 at 12 hrs after infection. The GFP antigen was clearly and robustly expressed by MSCs without nuclei indicating viral replication and antigen production in enucleated cells. Scale bar=50 μm. FIG. 11B. High magnification epifluorescent image of an MSC-derived cell without nucleus infected with VSV-GFP (arrowheads) at MOI 0.1 at 12 hours after infection. The cytoplast was also stained for F-actin filaments using rhodamine phalloidin (arrows) and the nuclear stain DAPI to illustrate the lack of the nucleus.

FIG. 12A-12D illustrate Epifluorescent microscopy images (FIG. 12A) of MSC and MSC without nucleus infected with oHSV encoding GFP antigen at MOI 0.05 at 48 hrs after infection. MSCs without nuclei (cytoplast) were generated from MSCs 18 hrs after inoculation with oHSV-GFP. Scale bar=50 μm. FIG. 12B illustrates that MSCs or MSCs without nuclei expressing lifeact-RFP were infected with 0.05 MOI of the oncolytic herpes simplex virus encoding GFP (oHSV-GFP) then injected into established U87 glioblastoma tumors growing in Nude mice. Images were taken 7 days after the injection. Both MSCs and MSCs without nuclei delivered oHSV to tumor cells as indicated by the strong GFP signal. It was notable that very few MSCs without nuclei were detected in the tumor after 7 days, whereas a large number of MSCs were present in the center (injection site) and at the outer edge of the growing tumor. FIG. 12C is a bar graph showing percentage of GFP-covered tumor area, which represents the portion of tumor cells infected by MSCs or MSCs without nuclei carrying the oHSV-GFP virus. FIG. 12D is a graph showing the increased ratio of CD8+ effector T cells present in established glioblastoma tumors treated with combination of IL-12 (adjuvant) engineered MSCs without nuclei and oHSV engineered MSCs without nuclei compared to PBS injected controls.

FIG. 13A-13B illustrate enucleated mesenchymal stromal cells (MSCs) (cytoplasts) readily uptake cell permeable antigen peptides. FIG. 13A shows MSCs (left) and enucleated MSCs (cytoplast) (right) incubated with 100 μM of the cell-permeable antigen peptide (Arg)9-FAM (6-Carboxyfluorescein, FAM-Arg-Arg-Arg-Arg-Arg-Arg-Arg-Arg-Arg-OH). Scale bar=50 μm. Arrows indicate Hoechst stained nuclei, arrowheads indicate positive (Arg)9-FAM. FIG. 13B illustrates bar graphs represents relative fluorescence intensity measured in Image.J. Corrected Total Cell Fluorescence=Integrated Density−(Area of selected cell X Mean fluorescence of background readings). Mean±SEM; n=10.

### DETAILED DESCRIPTION

Disclosed herein are compositions and kits, and methods of their use to treat or prevent pathogenic infections (e.g., viral, fungal, parasite, bacterial) or a disease or condition associated with such pathogenic infections. The compositions of the present disclosure comprise cytoplasts, which are enucleated cells engineered to contain, and in some cases, produce a therapeutic agent that is effective to treat the disease or the condition associated with a pathogenic infection, and/or prevent the pathogenic infection. In some embodiments, the therapeutic agent described herein may be a vaccine (e.g., attenuated viral antigen), a virus-targeting agent effective to treat acute viral infections, or combinations of the two. In some embodiments, the cytoplast may also be engineered to trap pathogens (e.g., in vivo) and inactivate them to treat acute infections and prevent further infection. In some embodiments, the pathogens are one or more viruses, such as coronavirus.

Existing cell-based therapies have many shortcomings. Development of effective cell-based therapeutics often requires genetic engineering and the introduction of new genetic material into the genome of cells ex vivo. However, this process can introduce dangerous mutations into the genome that produce cancer and other life-threatening diseases, especially if the engineered cells permanently engraft into the body or fuse with host cells. Another significant problem with many existing cell-based therapeutics is that after delivery to the body, the cells proliferate uncontrollably and can permanently engraft into the body, which can be life-threatening. Also, the lack of cell control after administration to the subject can make the delivery of precise doses of therapeutic cells and their bioactive products difficult (e.g., poor pharmacokinetics). Thus, there exists a need for a safe and controllable cell-based therapy to deliver therapeutic agents or other biomolecules.

Prior to patient or subject delivery, traditional cell-based therapeutics are commonly modified or genetically altered ex vivo to generate desirable cellular and therapeutic functions. However, when these cells are introduced into the subject, the new host environment can significantly reprogram and negatively alter, or otherwise render them ineffective. Thus, there is a need for a more predictable cell-based therapy that cannot respond to reprogramming and detrimental external signals.

Cell-based therapies that exist today are limited by the amount of DNA-damaging/gene targeting agents can be loaded into them for delivery to subjects as a therapeutic against cancer or other diseases. This includes, but is not limited to, DNA-damaging chemotherapeutic drugs, DNA-integrating viruses, oncolytic viruses, and gene therapy applications/delivery including, but not limited to, cluster regularly interspaced short palindromic repeats (CRISPR), small clusters of Cas (CRISPR/Cas system), and plasmids. Thus, there is a need for a cell-based therapy without such limitations, which may be an ideal platform delivering high doses of cytotoxic therapeutic agents.

There are several advantages to delivering a therapeutic agent to a subject using the cytoplasts of the instant disclosure. Unlike conventional cell-based therapies that transfer DNA from their nuclei (e.g., nuclear-encoded genes or foreign or mutant DNA) to host cells unintentionally, the cytoplast of the present disclosure are unable to do so without a nucleus. Additionally, delivery of the therapeutic agent to the subject using the cytoplasts described herein is controllable and finite (e.g., 14 days or fewer), at least because, without a nucleus, the cytoplasts cannot proliferate or differentiate into other cell types. The cytoplasts of the present disclosure may, in the absence of a nucleus, express and/or secrete the therapeutic agent or other biomolecules described herein, as well as migrate or home to a target cell or target tissue or environment in vivo. This is achieved, at least in part, by enucleating a parent cell using the methods described herein such that the resulting cytoplast retains the organelles from the parent cell that are sufficient for normal biological function (e.g., protein production/secretion, cell motility, chemokine sensing, and like). Even when delivered to a subject systemically, the cytoplasts described herein deliver the therapeutic agent to a target tissue or a target cell in the subject (e.g., lymph tissue, lung tissue) efficiently and effectively in a manner that is safe and controllable. Moreover, manufacturing large quantities of conventional cell-based therapies is time intensive and expensive, which limits their clinical applications. Although, it is thought that using immortalized cells containing nuclei (e.g., hTERT) to increase manufacturing capabilities could increase manufacturing scale and lower manufacturing costs, there are concerns that immortalized cells are prone to chromosomal abnormalities and promote tumor or ectopic tissue formation, rendering them unsafe for clinical applications. By enucleating such cells, or any cell type, according to the embodiments of the instant disclosure, increased scale and lower costs associated with manufacturing the cytoplasts may be achieved, while mitigating the risks to human health posed by conventional cell-based therapies.

The improved manufacturing scale and cost, safety profile, and efficiency of the compositions described herein have important benefits for vaccine development. The methods for producing the compositions described herein are faster than conventional vaccine development timelines, which usually require the isolation and purification of the vaccine (e.g., antigen, mRNA) from the producer cell line. By contrast, cytoplasts of the present disclosure are engineered to continuously produce the anti-viral composition, obviating the need for isolation and purification of the vaccine. At the point of need, the compositions described herein may be administered systemically (e.g., inhalation), rather than by intramuscular injection, avoiding a need for a medical facility to administer the vaccine and improving patient experience. Due to the ability of the cytoplast to rapidly home to the lymph tissue (or other target tissue), the vaccine may be deployed to the lymphatic system of a subject in a fraction of the time it would take certain conventional cell-based therapies (e.g., exosomes) administered systemically. In addition, the small size of the cytoplast (e.g., about 8 micrometers) ensures that the cytoplasts are not trapped small openings in the vasculature and tissue parenchyma, thereby improving biodistribution as compared with conventional cell-based therapies. Cytoplasts disclosed herein, may be engineered to express virtually any type of vaccine or anti-viral agent (e.g., anti-viral and/or neutralizing antibody) to fight an active infection as well as prevent future infections. In addition, the cytoplasts described herein may be engineered to express more than one type of vaccine (e.g., against more than one type of pathogen), enabling a panel of vaccines to be administered to a subject in a single dosage form. This is particularly beneficial for rapidly evolving pathogens (e.g., SARS-CoV-2), which may require multiple vaccines in the future for an effective immunization strategy.

The cytoplasts disclosed here are an off-the-shelf solution to an urgent medical need. The cytoplasts may be engineered before or after enucleation to express targeting moieties (e.g., homing receptors), immune-evading moieties (e.g., “don't eat me” signaling peptides), among other biomolecules sufficient to target the cytoplast to the lymph tissue without risk of clearance by the immune system before they get there. The cytoplasts may be cryopreserved, cryo-hibernated, or cryodesiccated, and stored for long periods of time with their biological activity slowed or stopped. When there is an urgent medical need, the biological function of the cytoplasts may be restored (e.g., thawing, rehydrating), and remain viable for up to 5 days for further engineering (e.g., to express a vaccine or anti-viral agent) as needed before delivery. Such biological functions include, but are not limited to expression of therapeutic surface proteins, immune stimulating antigens, or receptors, secrete cytokines, hormones, or proteins, release of exosomes, shedding membrane particles, stimulate the immune system through death processes, or create tunneling nanotubes. The cytoplasts of the instant disclosure may be frozen and thawed multiple times during the manufacturing and distribution process, without negatively impacting the cytoplast intended function, making them an ideal platform for a rapid vaccine deployment.

In some embodiments, the cytoplasts of the instant disclosure can be therapeutic without being engineered to produce or deliver an exogenous vaccine or other biomolecule described herein. For example, an unmanipulated cytoplast itself can have therapeutic properties when delivered into a patient or subject, such as for example a cytoplast derived from a cell obtained from a subject immune to a pathogen of interest, similar to a convalescent plasma therapy approach. Such cell may naturally produce neutralizing antibodies that block pathogen-host receptor engagement. In some embodiments, an unmanipulated cytoplast can produce any one of the therapeutic agents or biomolecules described herein naturally, which may be used to achieve a therapeutic effect in a subject in need thereof.

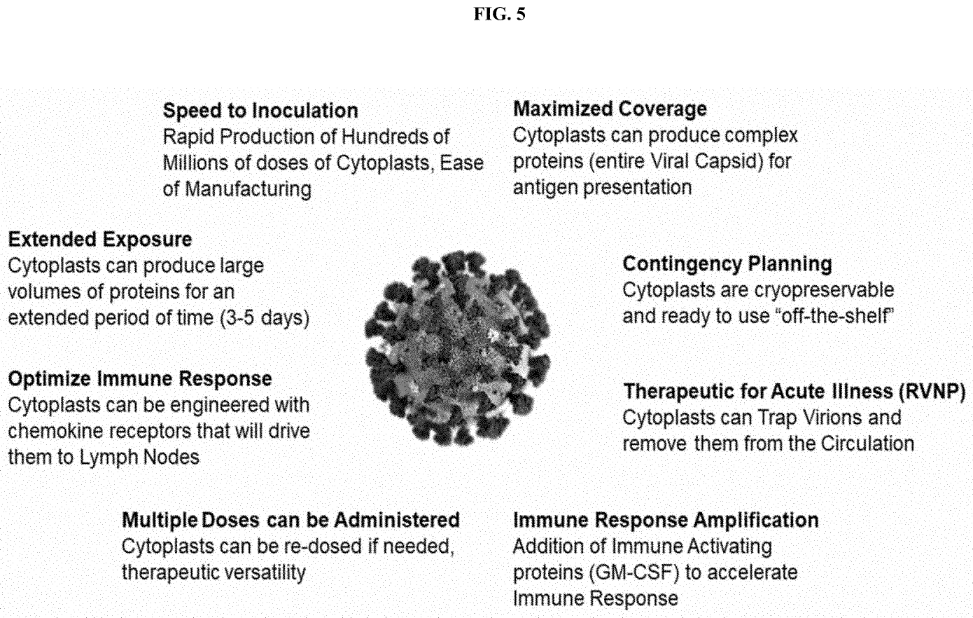

Non-limiting examples of the many benefits of the rapid vaccine platform described herein are provided in FIG. 5. The production of cytoplasts may be scaled up rapidly, where hundreds of millions cytoplasts engineered to express viral antigen may be manufactured with ease and may be stored until needed. The cytoplasts described herein, in addition to being engineered to express viral antigen, may act as a trap. Such technical feature allows the engineered cytoplast to be infected by a pathogen, thus sequestering the pathogen and preventing the pathogen from infecting other cells. For example, the cytoplast described herein can be engineered to express ACE2 receptor to be infected by a SARS-CoV-2 virus expressing the Spike protein. Upon infection, the SARS-CoV-2 virus is trapped in the cytoplast may no longer replicate. The infected cytoplast may be targeted by the immune system for degradation. The cytoplast may be engineered to express chemokine receptor to home the cytoplast to target tissue or microenvironment such as lymph node.

Provided here are compositions, methods, and kits for the prevention or treatment of pathogenic infections in a subject. In some embodiments, the pathogenic infection is a viral infection, such as infection of coronavirus or influenza virus. In some embodiments, the pathogenic infection is a bacterial infection. Disclosed herein are cytoplasts that are engineered to express an anti-viral composition that are suitable to prevent viral infection or outbreak, or treat acute infections. When delivered to a subject, the cytoplast delivers the anti-viral composition to a target tissue either by presenting the anti-viral composition on the surface of the cytoplast or by secreting the anti-viral composition into extracellular space surrounding the target tissue.

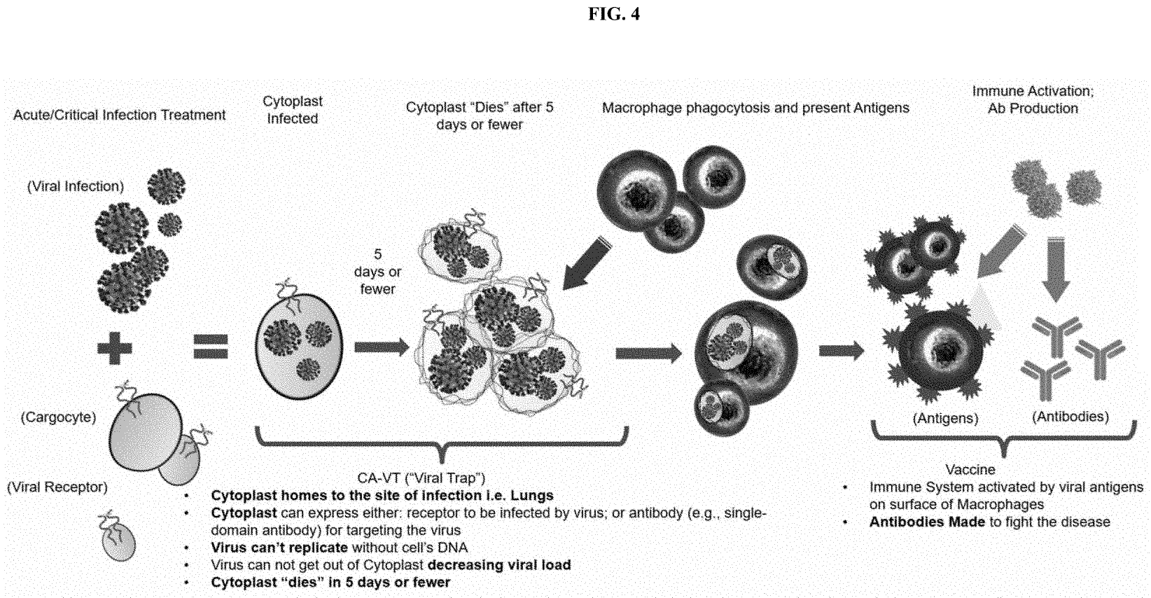

In some embodiments, the cytoplasts of the present disclosure are also suitable for trapping pathogens in a subject by permitting infection of the cytoplast by the pathogen and preventing propagation of the pathogen in vivo. As shown in FIG. 4, the cytoplast described herein can express a viral receptor that can be recognized by the pathogen, promoting infection of the cytoplast. The pathogen, upon infecting the cytoplast, is sequestered in the cytoplast unable to replicate or propagate in the absence of a nuclear genome. After 5 days or fewer, the cytoplast is cleared from the subject using natural processes of phagocytosis. In some embodiments, the cytoplast activates the immune system to accelerate clearance of the virus in the subject. At least one advantage to the cytoplasts disclosed herein for preventing the propagation of a pathogen in vivo is that they lack a nucleus containing genetic information necessary for many pathogens to replicate.

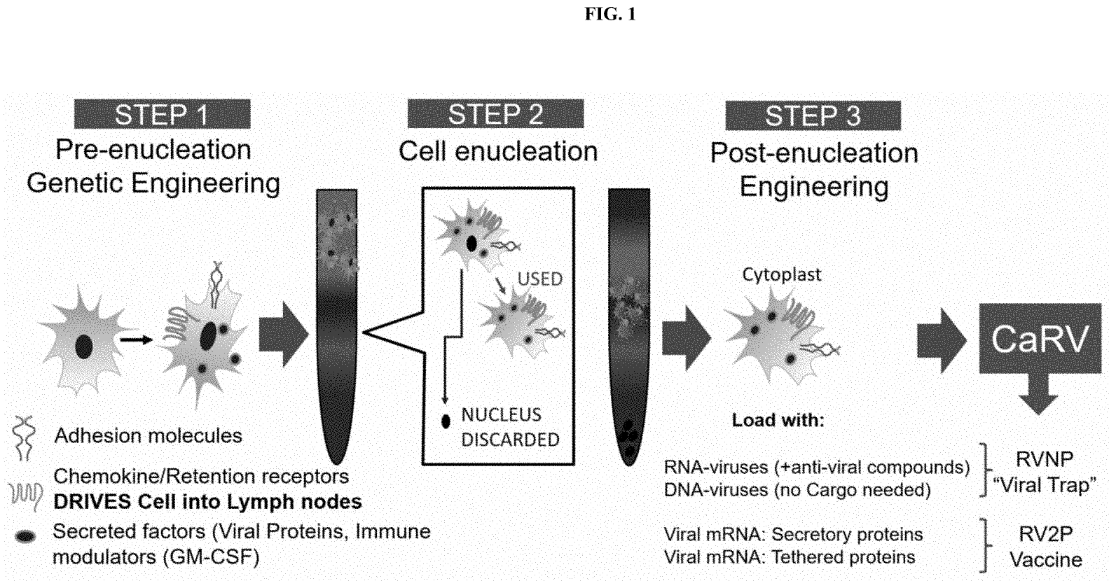

Referring to FIG. 1, in some embodiments, cells (e.g., stem cells) that are genetically engineered prior to enucleation to express adhesion molecules, chemokine or retention receptors or both, that target a target cell or tissue, such as the lymph tissue (e.g., lymph nodes) or the lung tissue in a subject (STEP 1). Next, the engineered cells are enucleated using the methods described herein to produce the cytoplasts (STEP 2). The cytoplasts may then be engineered to express and, in some embodiments, secrete a vaccine or other biomolecule (e.g., therapeutic agent, neutralizing antibody), and/or immune modulators (e.g., immune activators) to enhance the adaptive immune response in the subject (STEP 3). Cytoplasts are further engineered as needed depending on the intended function. The resulting cytoplasts may be used as a trap for viral trap or to deploy a vaccine. In the non-limiting example of the viral trap, the cytoplast may not be engineered with a therapeutic (e.g., vaccine) payload. Although, in some cases, it may be advantageous to express and/or secrete neutralizing antibodies against the pathogen of interest to prevent future infections by the virus. In some embodiments, the virus in this example is a coronavirus, such as SARS-CoV-2. However, the workflow in FIG. 1 may be applicable to any pathogen described herein, including bacterial pathogens (e.g., Bacillus anthracis) or toxins posing a significant risk to human health.

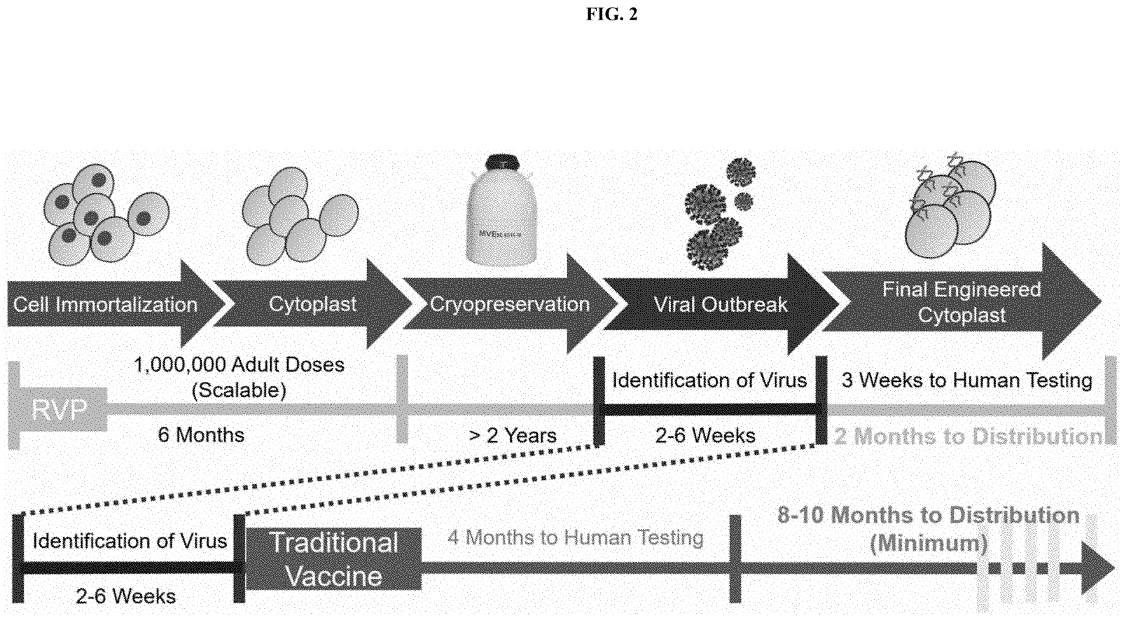

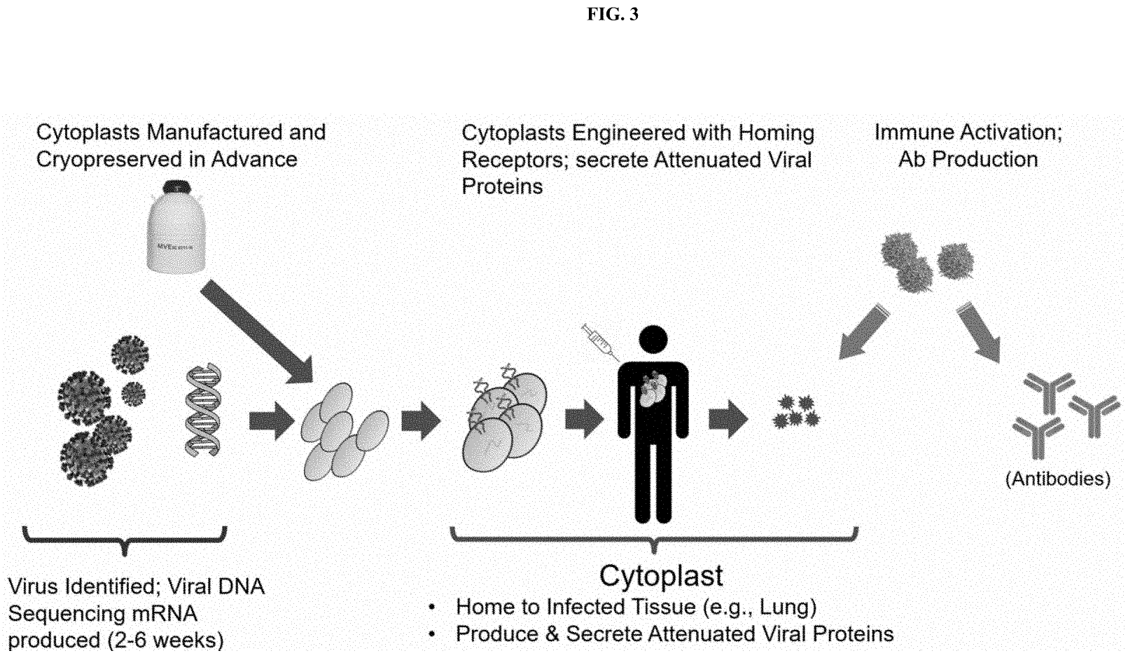

The process of manufacturing the cytoplasts of the present disclosure from identification of a new pathogen (e.g., a virus) to distribution is roughly 2 months, as compared with traditional vaccine development, which is 12 months or longer. As shown in FIG. 2, the cytoplasts of the present disclosure may be prepared in advance of the viral outbreak and cryopreserved for a length of time. This means, the cytoplasts of the present disclosure (e.g., engineered to express the homing receptors, immune activators) may be rapidly deployed to address the next viral outbreak. Referring to FIG. 3, the cytoplasts that were prepared in advance and cryopreserved are engineered to secrete attenuated viral proteins. When administered to a subject in need thereof, the cytoplasts drive immune activation and production of neutralizing antibodies against the virus in the subject.

Methods of producing cytoplasts of the present disclosure are provided. In some embodiments, cells can be treated with cytochalasin B to soften the cortical actin cytoskeleton. The nucleus can then be physically extracted from the cell body by highspeed centrifugation in gradients of Ficoll to generate a nucleus-free (enucleated) cytoplast. Because cytoplast and intact nucleated cells sediment to different layers in the Ficoll gradient, cytoplasts can, in some embodiments, be easily isolated and prepared for therapeutic purposes or fusion to other cells (nucleated or enucleated). The enucleation process can be clinically scalable to process tens of millions of cells.

Disclosed herein are methods of using or delivering the cytoplasts of the present disclosure. The cytoplasts can be used as a homing vehicle to deliver clinically relevant cargos/payloads to treat healthy individuals (e.g., to improve energy, recovery from exercise, or to deliver natural products) or various diseases (e.g., any of the diseases described herein). For example, cytoplasts may be used to deliver supplements, anti-aging factors, preventative treatments, and the like to healthy individuals, e.g., individuals who have not been diagnosed with a specific disorder for which the delivered therapeutic is effective.

Also, provided herein are kits that include any composition described herein. For example, a kit can include instructions for using any of the compositions or methods described herein. In some embodiments, the kits can include at least one dose of any of the compositions described herein.

### I. COMPOSITIONS

Provided herein are compositions useful for treating or preventing a pathogen-associated disease or condition in a subject. In some embodiments, the compositions disclosed herein comprise a cytoplast (e.g., an enucleated cell) engineered to express an active agent suitable for the treatment or the prevention of a pathogen-associated disease or condition. In some embodiments, the pathogen-associated disease or condition is a viral infection, such as a coronavirus infection. In some embodiments, cytoplast is engineered to express an anti-viral composition, such as an attenuated viral antigen or anti-viral antibody, or a combination thereof. In some embodiments, the cytoplast comprises the anti-viral composition at the surface of the cytoplast (e.g., antigen presentation). In some embodiments, the anti-viral composition is secreted by the cytoplast into extracellular space at a target tissue. In some embodiments, the cytoplast is engineered to capture or trap a pathogen in vivo by permitting infection of the cytoplast and preventing propagation of the pathogen in vivo, thereby treating an acute pathogenic infection, or pathogen-associate disease or condition.

The cytoplasts described herein are engineered to have a limited or defined (e.g., known, or programmable) life span. The cytoplasts described herein have a reduced size compared to cells in some other cell-based therapies (e.g., exosomes, red blood cells, adoptive cell therapies), which In some embodiments, improves biodistribution.

The cytoplasts described herein maintain viability following cryohibernation or cryopreservation, making them uniquely suitable for widespread adoption as a platform for drug delivery. Cryopreservation includes cooling or freezing, and storing, in the short-term or long-term, biological material (e.g., cells, cytoplasts) at very low temperatures (e.g., −80° C. in solid CO₂, −196° C. in liquid nitrogen, etc.). Cryohibernation includes short-term cooling and storing of biological material (e.g., cells, cytoplasts) in suspended animation, at non-freezing temperatures, such as, e.g., at 4° C. Cryohibernation of cytoplasts can be advantageous for one or more of the following reasons: cryohibernation is less labor-intensive than cryopreservation, and cytoplasts that have undergone cryohibernation can be transported (e.g., shipped). In some embodiments, the cytoplast is cryopreserved. In some embodiments, the cytoplast is cryohybernated. Following removal of the cytoplast from cryohybernation or cryopreservation, the cytoplasts may be used in accordance with the methods described herein. In some embodiments, the cytoplasts are viable for at least or about 24 hours, 48 hours, 72, or any increment of time between 24 and 72 hours following removal from cryohybernation or cryopreservation. In some embodiments, the cytoplasts are viable for between about 24 and about 48 hours. In some embodiments, the cytoplasts are viable for between about 48 and about 72 hours. In some embodiments, viability is measured using trypan blue dye exclusion as described herein. In some embodiments, viability is measured using Annexin-5 cell surface staining as described herein.

The cytoplasts described herein are extensively engineered, to best suit a given therapeutic application. For example, the cytoplasts are engineered (e.g., with cell-surface receptors) that increase infection of the cytoplast by a target pathogen. In some embodiments, cytoplasts are engineered to express an attenuated viral antigen for use as a vaccine or an anti-viral antibody for use in treating acute viral infections. In another example the cytoplasts are engineered to produce or express a protein that specifically targets difficult tissues (e.g., muscle) and an active agent such as an attenuated viral antigen or anti-viral antibody. In addition, in some embodiments, the cytoplasts are engineered with immune evading moieties (e.g., CD34+) to avoid an antigenic response in the host. Cytoplasts are also engineered to express cell-surface receptors (e.g., adhesion molecules, chemokine receptors) used for cellular homing, chemokine sensing, and other biological functions that are essential to targeting damaged tissue in a predominately affected area.

In some embodiments, a cytoplast has a defined life span of less than 1 hour to 14 days (e.g., less than 1 hour to 1 hour, less than 1 hour to 6 hours, 6 hours to 12 hours, 12 hours to 1 day, 1 day, 2 days, 3 days, 4 days, 5, days, 6 days, 7 days, 8 days, 9 days, 10 days, 11 days, 13 days, 14 days, 1 to 14 days, 1 to 12 days, 1 to 10 days, 1 to 9 days, 1 to 8 days, 1 to 7 days, 1 to 6 days, 1 to 5 days, 1 to 4 days, 1 to 3 days, 1 to 2 days, 2 to 14 days, 2 to 12 days, 2 to 10 days, 2 to 8 days, 2 to 7 days, 2 to 6 days, 2 to 5 days, 2 to 4 days, 2 to 3 days, 3 to 14 days, 3 to 12 days, 3 to 10 days, 3 to 8 days, 3 to 7 days, 3 to 6 days, 3 to 5 days, 3 to 4 days, 4 to 14 days, 4 to 12 days, 4 to 10 days, 4 to 8 days, 4 to 7 days, 4 to 6 days, 4 to 5 days, 4 to 7 days, 5 to 14 days, 5 to 12 days, 5 to 10 days, 5 to 8 days, 5 to 7 days, 5 to 6 days, 6 to 14 days, 6 to 12 days, 6 to 10 days, 6 to 8 days, 6 to 7 days, 7 to 14 days, 7 to 12 days, 7 to 10 days, 7 to 8 days, 8 to 14 days, 8 to 12 days, 8 to 10 days, 10 to 14 days, 10 to 12 days, 12 to 14 days, less than 14 days, less than 12 days, less than 10 days, less than 8 days, less than 7 days, less than 6 days, less than 5 days, less than 4 days, less than 3 days, less than 2 days, less than 1 day, less than 12 hours, or less than 6 hours). In some embodiments, the lifespan of a population of cytoplasts can be evaluated by determining the average time at which a portion of the cytoplast population (e.g., at least 50%, at least 60% at least 70%, at least 75%, at least 80%, at least 85%, at least 90%, at least 95%, or at least 98% of the population) is determined to be dead. Cell death can be determined by any method known in the art. In some embodiments, the viability of cytoplasts, e.g., at one or more time points, can be evaluated by determining whether morphometric or functional parameters are intact (e.g. by trypan-blue dye exclusion, evaluating for intact cell membranes, evaluating adhesion to plastics (e.g., in adherent cytoplasts), evaluating cytoplast migration, negative staining with apoptotic markers, and the like). In some embodiments, the life span of a cytoplast may be related to the life span of the cell from which it was obtained. For example, in some embodiments, a cytoplast obtained from a macrophage may live 12 to 24 hours.

In some embodiments, a cytoplast is at least or equal to 1 μm in diameter. In some embodiments, a cytoplast is greater than 1 μm in diameter. In some embodiments, a cytoplast is 1-100 μm in diameter (e.g., 1-90 μm, 1-80 μm, 1-70 μm, 1-60 μm, 1-50 μm, 1-40 μm, 1-30 μm, 1-20 μm, 1-10 μm, 1-5 μm, 5-90 μm, 5-80 μm, 5-70 μm, 5-60 μm, 5-50 μm, 5-40 μm, 5-30 μm, 5-20 μm, 5-10 μm, 10-90 μm, 10-80 μm, 10-70 μm, 10-60 μm, 10-50 μm, 10-40 μm, 10-30 μm, 10-20 μm, 10-15 μm 15-90 μm, 15-80 μm, 15-70 μm, 15-60 μm, 15-50 μm, 15-40 μm, 15-30 μm, 15-20 μm). In some embodiments, a cytoplast is 10-30 μm in diameter. In some embodiments, the diameter of a cytoplast is between 5-25 μm (e.g., 5-20 μm, 5-15 μm. 5-10 μm, 10-25 μm, 10-20 μm, 10-15 μm, 15-25 μm, 15-20 μm, or 20-25 μm. In some embodiments, a cytoplast is not an exosome. Without being bound by any particular theory, it is believed that, In some embodiments, some cytoplasts can advantageously be small enough to allow for better biodistribution or to be less likely to be trapped in the lungs of a subject.

In some embodiments, cytoplasts can be applied to or cultured with cells (e.g., xenocultured cells) to alter their properties. For example, in some embodiments, cytoplasts (e.g., unmanipulated cytoplasts or engineered cytoplasts) can upregulate health-promoting factors in xenocultured cells, and in some embodiments, the xenocultured cells can be returned to the subject from which they were taken.

A. Cells

Provided herein are cells and cell lines that are engineered to produce the cytoplasts of the present disclosure. The cytoplast may be derived from a corresponding parent cell, such as a nucleated parent cell. Non-limiting examples of parent cells include an immortalized cell, a cancer cell (e.g., any cancer cell), a primary (e.g., host-derived) cell, or a cell line. In some embodiments, the parent cell is derived from a cell is immortalized using suitable methods, such as those described in Huang et al., J. Exp. Clin. Med. 2010 October. 221 2(5):202-217. In some embodiments, the cytoplast is derived from a parent cell using suitable methods provided in U.S. patent application Ser. No. 16/715,859, which is hereby incorporated by reference in its entirety.

In some embodiments, the cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant, an algal cell, a fungal cell, an animal cell, a cell from an invertebrate animal, a cell from a vertebrate animal, a cell from a mammal (e.g., a pig, a cow, a goat, a sheep, a rodent, a rat, a mouse, a non-human primate, a human, etc.), and etcetera. In some embodiments, the cell is a somatic cell. In some embodiments, the cell is a stem cell or a progenitor cell. In some embodiments, the cell is a mesenchymal stem or progenitor cell. In some embodiments, the cell is a mesenchymal stromal cell. A cell can originate from any organism having one or more cells.

Some non-limiting examples of cells include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell). In some embodiments, the cell is a somatic cell. In some embodiments, the cell is a stem cell or a progenitor cell. In some embodiments, the cell is a mesenchymal stem or progenitor cell. In some embodiments, the cell is a hematopoietic stem or progenitor cell. In some embodiments, the cell is a muscle cell, a skin cell, a blood cell, or an immune cell. Other exemplary cells can include lymphoid cells, such as B cell, T cell (Cytotoxic T cell, Natural Killer T cell, Regulatory T cell, T helper cell), Natural killer cell, cytokine induced killer (CIK) cells; myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex -hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

Non-limiting examples of eukaryotic cells include mammalian (e.g., rodent, non-human primate, or human), non-mammalian animal (e.g., fish, bird, reptile, or amphibian), invertebrate, insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. In some embodiments, the nucleated cell is a primary cell. In some embodiments, the nucleated cell is an immune cell (e.g., a lymphocyte (e.g., a T cell, a B cell), a macrophage, a natural killer cell, a neutrophil, a mast cell, a basophil, a dendritic cell, a monocyte, a myeloid-derived suppressor cell, an eosinophil). In some embodiments, the nucleated cell is a phagocyte or a leukocyte. In some embodiments, the nucleated cell is a stem cell (e.g., an adult stem cell (e.g., a hematopoietic stem cell, a mammary stem cell, an intestinal stem cell, mesenchymal stem cell, an endothelial stem cell, a neural stem cell, an olfactory adult stem cell, a neural crest stem cell, a testicular cell), an embryonic stem cell, an inducible pluripotent stem cell (iPS)). In some embodiments, the nucleated cell is a progenitor cell. In some embodiments, the nucleated cell is from a cell line. In some embodiments, the nucleated cell is a suspension cell. In some embodiments, the nucleated cell is an adherent cell. In some embodiments, the nucleated cell is a cell that has been immortalized by expression of an oncogene. In some embodiments, the nucleated cell is immortalized by the expression of human telomerase reverse transcriptase (hTERT) or any oncogene. In some embodiments, the nucleated cell is a patient or subject derived cell (e.g., an autologous patient-derived cell, or an allogenic patient-derived cell). In some embodiments, the nucleated cell is transfected with a vector (e.g., a viral vector (e.g., a retrovirus vector (e.g., a lentivirus vector), an adeno-associated virus (AAV) vector, a vesicular virus vector (e.g., vesicular stomatitis virus (VSV) vector), or a hybrid virus vector), a plasmid) before the nucleated cell is enucleated using any of the enucleation techniques described herein and known in the art.

In some embodiments, the cytoplast can be derived from a cell autologous to the subject. In some embodiments, the cytoplast can be derived from a cell allogenic to the subject.

In some embodiments, the cytoplast is derived from an immune cell. In some embodiments, the cytoplast is derived from a natural killer (NK) cell, a neutrophil, a macrophage, a lymphocyte, a fibroblast, an adult stem cell (e.g., hematopoietic stem cell, a mammary stem cell, an intestinal stem cell, a mesenchymal stem cell, a mesenchymal stromal cell, an endothelial stem cell, a neural stem cell, an olfactory adult stem cell, a neural crest stem cell, a skin stem cell, or a testicular cell), a mast cell, a basophil, an eosinophil, or an inducible pluripotent stem cell.

In some embodiments, prior to enucleation, two or more cells (e.g., any of the cells disclosed herein) are fused by any method disclosed herein or known in the art. Enucleation of the fusion product can result in a cytoplast.

In some embodiments, a first cytoplast is fused to a cell or second cytoplast. In some embodiments, the cell is any nucleated (e.g., a mammalian cell (e.g., a human cell, or any mammalian cell described herein), a protozoal cell (e.g., an amoeba cell), an algal cell, a plant cell, a fungal cell, an invertebrate cell, a fish cell, an amphibian cell, a reptile cell, or a bird cell). In some embodiments, the second cell is a synthetic cell. Accordingly, provided are methods of altering the behavior of a cell comprising fusing the cell with any of the cytoplasts described herein. Also provided herein are methods comprising administering to a subject a therapeutically effective amount of a cell to which a cytoplast has been fused.

In some embodiments, the second cytoplast is derived from the same type of cell as the first cytoplast. In some embodiments, the second cytoplast is derived from a different type of cell as the first cytoplast. In some embodiments, the second cytoplast contains or expresses at least one therapeutic DNA molecule, therapeutic RNA molecule, therapeutic protein, therapeutic peptide, small molecule therapeutic, therapeutic gene editing factor, a therapeutic nanoparticle, or another active agent that is the same as a therapeutic DNA molecule, therapeutic RNA molecule, therapeutic protein, therapeutic peptide, small molecule therapeutic, therapeutic gene editing factor, a therapeutic nanoparticle contained in or expressed by the first cytoplast. In some embodiments, the second cytoplast contains or expresses at least one therapeutic DNA molecule, therapeutic RNA molecule, therapeutic protein, therapeutic peptide, small molecule therapeutic, therapeutic gene editing factor, a therapeutic nanoparticle, or another active agent that is different from a therapeutic DNA molecule, therapeutic RNA molecule, therapeutic protein, therapeutic peptide, small molecule therapeutic, therapeutic gene editing factor, a therapeutic nanoparticle contained in or expressed by the first cytoplast. In some embodiments, a first cytoplast can be fused to a cell or to a second cytoplast using any method known in the art, for example, electrofusion or viral fusion using viral-based cell surface peptides.

In some embodiments, a cytoplast is not a naturally occurring enucleated cell. In some embodiments, a cytoplast is not obtained from a cell that naturally undergoes enucleation. In some embodiments, a cytoplast is not a cell that has been enucleated by in the body of a subject. In some embodiments, a cytoplast is not obtained from a cell that would be enucleated by in the body of a subject. In some embodiments, a cytoplast is not obtained from an erythroblast. In some embodiments, a cytoplast is obtained from a cell that maintains a nucleus over its lifespan (e.g., in the absence of manipulations such as enucleation as described herein). In some embodiments, a cytoplast is not a cell that is found in a subject as an anucleate cell (e.g., a red blood cell (erythrocyte), a platelet, a lens cell, or an immediate nucleated precursor thereof). In some embodiments, a cytoplast includes one or more components selected from the group consisting of an endoplasmic reticulum, a Golgi apparatus, mitochondria, ribosomes, proteasomes, or spliceosomes. In some embodiments, a cytoplast is characterized by one or more of the following features: adhesion, tunneling nanotube formation, actin-mediated spreading (2D and/or 3D), migration, chemoattractant gradient sensing, mitochondrial transfer, mRNA translation, protein synthesis, and secretion of exosomes and/or other bioactive molecules. In some embodiments, a cytoplast is characterized by an ability to secrete proteins (e.g., using exosomes). In some embodiments, a cytoplast has been enucleated ex vivo. In some embodiments, a cytoplast has been enucleated in vitro. In some embodiments, a cytoplast has been physically enucleated (e.g., by centrifugation). In some embodiments, a cytoplast is an engineered enucleated cell. In some embodiments, a cytoplast is not a red blood cell. In some embodiments, a cytoplast does not contain hemoglobin. In some embodiments, a cytoplast does not have a bi-concave shape.

In some embodiments, a cytoplast is not obtained from an erythroblast. In some embodiments, a cytoplast is obtained from a cell that would not become a red blood cell (RBC). Unlike RBCs cytoplasts can be viable cell-like entities that can retain many active biological processes and all cellular organelles (e.g., ER/Golgi, mitochondrial, endosome, lysosome, cytoskeleton, etc.). Thus, cytoplasts can function like nucleated cells and exhibit critical biological functions such as adhesion, tunneling nanotube formation, actin-mediated spreading (2D and 3D), migration, chemoattractant gradient sensing, mitochondrial transfer, mRNA translation, protein synthesis, and secretion of exosomes and other bioactive molecules. One or more of these functions may not be exhibited by RBCs. Compared to RBCs, which are derived from erythroblasts, a cytoplast can be derived from any type of nucleated cell, including, but not limited to iPSC (induced pluripotent stem cells), any immortalized cell, stem cells, primary cells (e.g., host-derived cells), cell lines, any immune cell, cancerous cells, or from any eukaryotic cell. In some embodiments, a cytoplast is obtained from a lymphoid progenitor cell. In some embodiments, a cytoplast is obtained from a lymphocyte. In some embodiments, a cytoplast is obtained from a mesenchymal stem cell (e.g., from bone marrow). In some embodiments, a cytoplast is obtained from an endothelial stem cell. In some embodiments, a cytoplast is obtained from a neural stem cell. In some embodiments, a cytoplast is obtained from a skin stem cell.

B. Pathogens

The cytoplasts described herein and compositions containing the cytoplasts, in some embodiments, comprise biomolecules (e.g., vaccine, therapeutic agent, targeting moieties) that target and/or kill, or otherwise render inoperable, a pathogen. In some embodiments, the pathogen is a bacteria, a virus, a fungus, or a toxin. In some embodiments, the pathogen is naturally occurring. In some embodiments, the pathogen is synthetic.

In some embodiments, the pathogen is a virus. In some embodiments, the virus is an animal virus, a plant virus, a bacterial virus, or an archaeal virus. In some embodiments, the animal virus causes a disease or condition in the same or a different animal. In some embodiments, the virus is an RNA virus or a DNA virus. In some embodiments, the RNA or DNA virus is single-stranded or double-stranded. In some embodiments, the DNA or RNA virus is a positive-sense or a negative-sense virus.

In some embodiments, the double-stranded virus (dsDNA) virus is from the family: Myoviridae, Podoviridae, Siphoviridae, Alloherpesviridae, Herpesviridae, Malacoherpesviridae, Lipothrixviridae, Rudiviridae, Adenoviridae, Ampullaviridae, Ascoviridae, Asfaviridae, Baculoviridae, Bicaudaviridae, Clavaviridae, Corticoviridae, Fuselloviridae, Globuloviridae, Guttaviridae, Hytrosaviridae, Iridoviridae, Marseilleviridae, Mimiviridae, Nimaviridae, Pandoraviridae, Papillomaviridae, Phycodnaviridae, Plasmaviridae, Polydnaviruses, Polyomaviridae, Poxviridae, Sphaerolipoviridae, and Tectiviridae.

In some embodiments, the single-stranded (ssDNA) virus is from the family: Anelloviridae, Bacillariodnaviridae, Bidnaviridae, Circoviridae, Geminiviridae, Inoviridae, Microviridae, Nanoviridae, Parvoviridae, and Spiraviridae.

A DNA virus that contains both ss and ds DNA regions can be from the group of pleolipoviruses. In some embodiments, the pleolipoviruses include Haloarcula hispanica pleomorphic virus 1, Halogeometricum pleomorphic virus 1, Halorubrum pleomorphic virus 1, Halorubrum pleomorphic virus 2, Halorubrum pleomorphic virus 3, and Halorubrum pleomorphic virus 6.

In some embodiments, the dsRNA virus is from the family: Birnaviridae, Chrysoviridae, Cystoviridae, Endornaviridae, Hypoviridae, Megavirnaviridae, Partitiviridae, Picobirnaviridae, Reoviridae, Rotavirus and Totiviridae.

In some embodiments, the positive-sense ssRNA virus can be from the family: Alphaflexiviridae, Alphatetraviridae, Alvernaviridae, Arteriviridae, Astroviridae, Barnaviridae, Betaflexiviridae, Bromoviridae, Caliciviridae, Carmotetraviridae, Closteroviridae, Coronaviridae, Dicistroviridae, Flaviviridae, Gammaflexiviridae, Iflaviridae, Leviviridae, Luteoviridae, Marnaviridae, Mesoniviridae, Narnaviridae, Nodaviridae, Permutotetraviridae, Picornaviridae, Potyviridae, Roniviridae, Secoviridae, Togaviridae, Tombusviridae, Tymoviridae, and Virgaviridae.

In some embodiments, the negative-sense ssRNA virus can be from the family: Bornaviridae, Filoviridae, Paramyxoviridae, Rhabdoviridae, Nyamiviridae, Arenaviridae, Bunyaviridae, Ophioviridae, and Orthomyxoviridae.

Non-limiting examples of viruses include: Abelson leukemia virus, Abelson murine leukemia virus, Abelson's virus, Acute laryngotracheobronchitis virus, Adelaide River virus, Adeno associated virus group, Adenovirus, African horse sickness virus, African swine fever virus, AIDS virus, Aleutian mink disease parvovirus, Alpharetrovirus, Alphavirus, ALV related virus, Amapari virus, Aphthovirus, Aquareovirus, Arbovirus, Arbovirus C, arbovirus group A, arbovirus group B, Arenavirus group, Argentine hemorrhagic fever virus, Argentine hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, avian leukosis virus, avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus, B19 virus, Babanki virus, baboon herpesvirus, baculovirus, Barmah Forest virus, Bebaru virus, Berrimah virus, Betaretrovirus, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, Buggy Creek virus, bullet shaped virus group, Bunyamwera virus supergroup, Bunyavirus, Burkitt's lymphoma virus, Bwamba Fever, CA virus, Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, caprine arthritis virus, caprine encephalitis virus, Caprine Herpes Virus, Capripox virus, Cardiovirus, caviid herpesvirus 1, Cercopithecid herpesvirus 1, cercopithecine herpesvirus 1, Cercopithecine herpesvirus 2, Chandipura virus, Changuinola virus, channel catfish virus, Charleville virus, chickenpox virus, Chikungunya virus, chimpanzee herpesvirus, chub reovirus, chum salmon virus, Cocal virus, Coho salmon reovirus, coital exanthema virus, Colorado tick fever virus, Coltivirus, Columbia SK virus, common cold virus, contagious eethyma virus, contagious pustular dermatitis virus, Coronavirus, Corriparta virus, coryza virus, cowpox virus, coxsackie virus, CPV (cytoplasmic polyhedrosis virus), cricket paralysis virus, Crimean-Congo hemorrhagic fever virus, croup associated virus, Cryptovirus, Cypovirus, Cytomegalovirus, cytomegalovirus group, cytoplasmic polyhedrosis virus, deer papillomavirus, deltaretrovirus, dengue virus, Densovirus, Dependovirus, Dhori virus, diploma virus, Drosophila C virus, duck hepatitis B virus, duck hepatitis virus 1, duck hepatitis virus 2, duovirus, Duvenhage virus, Deformed wing virus DWV, eastern equine encephalitis virus, eastern equine encephalomyelitis virus, EB virus, Ebola virus, Ebola-like virus, echo virus, echovirus, echovirus 10, echovirus 28, echovirus 9, ectromelia virus, EEE virus, EIA virus, EIA virus, encephalitis virus, encephalomyocarditis group virus, encephalomyocarditis virus, Enterovirus, enzyme elevating virus, enzyme elevating virus (LDH), epidemic hemorrhagic fever virus, epizootic hemorrhagic disease virus, Epstein-Barr virus, equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Filovirus, Flanders virus, Flavivirus, foot and mouth disease virus, Fort Morgan virus, Four Corners hantavirus, fowl adenovirus 1, fowlpox virus, Friend virus, Gammaretrovirus, GB hepatitis virus, GB virus, German measles virus, Getah virus, gibbon ape leukemia virus, glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantaan virus, Hantavirus, hard clam reovirus, hare fibroma virus, HCMV (human cytomegalovirus), hemadsorption virus 2, hemagglutinating virus of Japan, hemorrhagic fever virus, hendra virus, Henipaviruses, Hepadnavirus, hepatitis A virus, hepatitis B virus group, hepatitis C virus, hepatitis D virus, hepatitis delta virus, hepatitis E virus, hepatitis F virus, hepatitis G virus, hepatitis nonA nonB virus, hepatitis virus, hepatitis virus (nonhuman), hepatoencephalomyelitis reovirus 3, Hepatovirus, heron hepatitis B virus, herpes B virus, herpes simplex virus, herpes simplex virus 1, herpes simplex virus 2, herpesvirus, herpesvirus 7, Herpesvirus ateles, Herpesvirus hominis, Herpesvirus infection, Herpesvirus saimiri, Herpesvirus suis, Herpesvirus varicellae, Highlands J virus, Hirame rhabdovirus, hog cholera virus, human adenovirus 2, human alphaherpesvirus 1, human alphaherpesvirus 2, human alphaherpesvirus 3, human B lymphotropic virus, human betaherpesvirus 5, human coronavirus, human cytomegalovirus group, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immodeficiency virus, human immodeficiency virus 1, human immunodeficiency virus 2, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I, human T lymphotropic virus II, human T lymphotropic virus III, Ichnovirus, infantile gastroenteritis virus, infectious bovine rhinotracheitis virus, infectious haematopoietic necrosis virus, infectious pancreatic necrosis virus, influenza virus A, influenza virus B, influenza virus C, influenza virus D, influenza virus pr8, insect iridescent virus, insect virus, iridovirus, Japanese B virus, Japanese encephalitis virus, JC virus, Junin virus, Kaposi's sarcoma-associated herpesvirus, Kemerovo virus, Kilham's rat virus, Klamath virus, Kolongo virus, Korean hemorrhagic fever virus, kumba virus, Kysanur forest disease virus, Kyzylagach virus, La Crosse virus, lactic dehydrogenase elevating virus, lactic dehydrogenase virus, Lagos bat virus, Langur virus, lapine parvovirus, Lassa fever virus, Lassa virus, latent rat virus, LCM virus, Leaky virus, Lentivirus, Leporipoxvirus, leukemia virus, leukovirus, lumpy skin disease virus, lymphadenopathy associated virus, Lymphocryptovirus, lymphocytic choriomeningitis virus, lymphoproliferative virus group, Machupo virus, mad itch virus, mammalian type B oncovirus group, mammalian type B retroviruses, mammalian type C retrovirus group, mammalian type D retroviruses, mammary tumor virus, Mapuera virus, Marburg virus, Marburg-like virus, Mason Pfizer monkey virus, Mastadenovirus, Mayaro virus, ME virus, measles virus, Menangle virus, Mengo virus, Mengovirus, Middelburg virus, milkers nodule virus, mink enteritis virus, minute virus of mice, MLV related virus, MM virus, Mokola virus, Molluscipoxvirus, Molluscum contagiosum virus, monkey B virus, monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Neethling virus, Nelson Bay virus, neurotropic virus, New World Arenavirus, newborn pneumonitis virus, Newcastle disease virus, Nipah virus, noncytopathogenic virus, Norwalk virus, nuclear polyhedrosis virus (NPV), nipple neck virus, O'nyong'nyong virus, Ockelbo virus, oncogenic virus, oncogenic viruslike particle, oncornavirus, Orbivirus, Orf virus, Oropouche virus, Orthohepadnavirus, Orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, parainfluenza virus, parainfluenza virus type 1, parainfluenza virus type 2, parainfluenza virus type 3, parainfluenza virus type 4, Paramyxovirus, Parapoxvirus, paravaccinia virus, Parvovirus, Parvovirus B19, parvovirus group, Pestivirus, Phlebovirus, phocine distemper virus, Picodnavirus, Picornavirus, pig cytomegalovirus-pigeonpox virus, Piry virus, Pixuna virus, pneumonia virus of mice, Pneumovirus, poliomyelitis virus, poliovirus, Polydnavirus, polyhedral virus, polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, pox virus, poxvirus, poxvirus variolae, Prospect Hill virus, Provirus, pseudocowpox virus, pseudorabies virus, psittacinepox virus, quailpox virus, rabbit fibroma virus, rabbit kidney vaculolating virus, rabbit papillomavirus, rabies virus, raccoon parvovirus, raccoonpox virus, Ranikhet virus, rat cytomegalovirus, rat parvovirus, rat virus, Rauscher's virus, recombinant vaccinia virus, recombinant virus, reovirus, reovirus 1, reovirus 2, reovirus 3, reptilian type C virus, respiratory infection virus, respiratory syncytial virus, respiratory virus, reticuloendotheliosis virus, Rhabdovirus, Rhabdovirus carpia, Rhadinovirus, Rhinovirus, Rhizidiovirus, Rift Valley fever virus, Riley's virus, rinderpest virus, RNA tumor virus, Ross River virus, Rotavirus, rougeole virus, Rous sarcoma virus, rubella virus, rubeola virus, Rubivirus, Russian autumn encephalitis virus, SA 11 simian virus, SA2 virus, Sabia virus, Sagiyama virus, Saimirine herpesvirus 1, salivary gland virus, sandfly fever virus group, Sandjimba virus, SARS virus, SDAV (sialodacryoadenitis virus), sealpox virus, Semliki Forest Virus, Seoul virus, sheeppox virus, Shope fibroma virus, Shope papilloma virus, simian foamy virus, simian hepatitis A virus, simian human immunodeficiency virus, simian immunodeficiency virus, simian parainfluenza virus, simian T cell lymphotrophic virus, simian virus, simian virus 40, Simplexvirus, Sin Nombre virus, Sindbis virus, smallpox virus, South American hemorrhagic fever viruses, sparrowpox virus, Spumavirus, squirrel fibroma virus, squirrel monkey retrovirus, SSV 1 virus group, STLV (simian T lymphotropic virus) type I, STLV (simian T lymphotropic virus) type II, STLV (simian T lymphotropic virus) type III, stomatitis papulosa virus, submaxillary virus, suid alphaherpesvirus 1, suid herpesvirus 2, Suipoxvirus, swamp fever virus, swinepox virus, Swiss mouse leukemia virus, TAC virus, Tacaribe complex virus, Tacaribe virus, Tanapox virus, Taterapox virus, Tench reovirus, Theiler's encephalomyelitis virus, Theiler's virus, Thogoto virus, Thottapalayam virus, Tick borne encephalitis virus, Tioman virus, Togavirus, Torovirus, tumor virus, Tupaia virus, turkey rhinotracheitis virus, turkeypox virus, type C retroviruses, type D oncovirus, type D retrovirus group, ulcerative disease rhabdovirus, Una virus, Uukuniemi virus group, vaccinia virus, vacuolating virus, varicella zoster virus, Varicellovirus, Varicola virus, variola major virus, variola virus, Vasin Gishu disease virus, VEE virus, Venezuelan equine encephalitis virus, Venezuelan equine encephalomyelitis virus, Venezuelan hemorrhagic fever virus, vesicular stomatitis virus, Vesiculovirus, Vilyuisk virus, viper retrovirus, viral haemorrhagic septicemia virus, Visna Maedi virus, Visna virus, volepox virus, VSV (vesicular stomatitis virus), Wallal virus, Warrego virus, wart virus, WEE virus, West Nile virus, western equine encephalitis virus, western equine encephalomyelitis virus, Whataroa virus, Winter Vomiting Virus, woodchuck hepatitis B virus, woolly monkey sarcoma virus, wound tumor virus, WRSV virus, Yaba monkey tumor virus, Yaba virus, Yatapoxvirus, yellow fever virus, and the Yug Bogdanovac virus.

In some embodiments, the virus is a coronavirus. In some embodiments, the coronavirus can be selected from the group consisting of: alphacoronavirus, betacoronavirus, deltacoronavirus, and gammacoronavirus. Examples of alphacoronavirus can include, but are not limited to, Bat coronavirus CDPHE15, Bat coronavirus HKU10, Human coronavirus 229E, Human coronavirus NL63, Miniopterus bat coronavirus 1, Miniopterus bat coronavirus HKU8, Mink coronavirus 1, Porcine epidemic diarrhea virus, Rhinolophus bat coronavirus HKU2, and Scotophilus bat coronavirus 512. Examples of betacoronavirus can include, but are not limited to, Betacoronavirus 1, Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus, Tylonycteris bat coronavirus HKU4. Examples of deltacoronavirus can include, but are not limited to, Bulbul coronavirus HKU11, Common moorhen coronavirus HKU21, Coronavirus HKU15, Munia coronavirus HKU13, Night heron coronavirus HKU19, Thrush coronavirus HKU12, White-eye coronavirus HKU16, Wigeon coronavirus HKU20. Examples of gammacoronavirus can include, but are not limited to, Avian coronavirus, Beluga whale coronavirus SW1. Additional examples of coronavirus can include MERS-CoV, SARS-CoV, and SARS-CoV-2. In some embodiments, the coronavirus can be SARS-CoV-2.

In some embodiments, the pathogen can: be easily disseminated or transmitted from person to person; result in high mortality rates and have the potential for major public health impact; and cause public panic and social disruption; and require special action for public health preparedness. Example of these pathogens can include Anthrax (Bacillus anthracis), Botulism (Clostridium botulinum toxin), Plague (Yersinia pestis), Smallpox (variola major), Tularemia (Francisella tularensis), or Viral hemorrhagic fevers, including Filoviruses (Ebola, Marburg) and Arenaviruses (Lassa, Machupo).

In some embodiments, the pathogen can: be moderately easy to disseminate; result in moderate morbidity rates and low mortality rates; and require specific enhancements of diagnostic capacity and enhanced disease surveillance. Example of these pathogens can include Brucellosis (Brucella species), Epsilon toxin of Clostridium perfringens, Food safety threats (e.g., Salmonella species, Escherichia coli O157:H7, or Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Viral encephalitis (alphaviruses, such as eastern equine encephalitis, Venezuelan equine encephalitis, and western equine encephalitis), or Water safety threats (e.g., Vibrio cholerae and Cryptosporidium parvum).

In some embodiments, the pathogen is an emerging pathogen with a sequence that is not yet identified. In some embodiments, the emerging pathogen has a potential for high morbidity and mortality rates and major health impact. Example of these pathogens can include Nipah virus and hantavirus.

In some embodiments, the pathogen can comprise a toxin. In some embodiments, the toxin can be secreted by any one of the pathogen described herein.

In some embodiments, the pathogen comprise a bacterium. In some embodiments, the bacterium may be a Gram-positive bacterium. In some embodiments, the bacterium is a Gram-negative bacterium. In some embodiments, the bacterium is a strain that is resistant to β-lactamase In some embodiments, the antigen is derived from Enterotoxigenic Escherichia coli (ETEC), Shiga toxin-producing Escherichia coli (STEC), Campylobacter jejuni, Pseudomonas aeruginosa, Acinetobacter baumannii, Streptococcus mutans, Helicobacter pylori, or Bacillus anthracis.

Exemplary list of pathogens and the diseases or conditions associated with these pathogens that can be treated with the enucleated cell, the composition, or the pharmaceutical composition described herein can be found in Tables 3-6.

TABLE 3
Exemplary virus and viral disease that may be treated or
vaccinated by the cytoplast

| Target | Disease |
|---|---|
| Respiratory syncytial virus (RSV) | RSV Infection |
| Rhesus monkey rotavirus (RV) | RV-induced diarrhea |
| Rhesus monkey RV serotype G3, | RV-induced diarrhea |
| strain RRV | |
| P domain VP1 capsid protein | Norovirus |
| H5 hemagglutinin | H5N1 influenza |
| HA1 hemagglutinin | H5N2 influenza |
| Nucleoprotein | Influenza A |
| Nsp9 | Porcine reproductive and |
| | respiratory syndrome |
| | virus (PRRSV) |
| Hepatitis C (HCV) E2 | HCV |
| glycoprotein | |
| NS3/4A | HCV genotype 3a |
| Retrovirus (Rev) | HIV-1 |
| CXCR4 | HIV-1 |
| Human glycophorin A | HIV (diagnostics) |
| HIV-1 Nef | HIV-1 |
| Nucleoprotein | Ebolavirus (diagnostics biothreat |
| | assays: MARSA) |
| Nucleoprotein prNΔ85 | Hantavirus (diagnostics) |
| H5N1 Influenza | H5N1 Influenza (diagnostics) |
| H3N2 | H3N2 Influenza (diagnostics) |
| HIV-1 virion infectivity | HIV monitoring (diagnostics) |
| factor (Vif) | |

TABLE 4
Exemplary bacterium and bacterial disease that may be treated or
vaccinated by the cytoplast

| Target | Disease |
|---|---|
| Lectin domain F18 fimbriae | Enterotoxigenic Escherichia coli |
| | (ETEC) and Shiga toxin-producing |
| | Escherichia coli (STEC) |
| F4 fimbriae | ETEC |
| FeaGac Adhesin of F4 fimbriae | ETEC |
| Flagella | Campylobacter jejuni |
| Flagella | Pseudomonas aeruginosa |
| Biofilm-associated protein | Acinetobacter baumannii |
| Streptococcus mutans strain | Streptococcus. mutans |
| HG982 | |
| TssM protein of type VI | Gram-negative bacteria |
| secretion system | |
| TEM-1 and Bcll ß-lactamase | ß-lactamase-resistant bacterial strains |
| UreC subunit of urease | Helicobacter pylori |

TABLE 5
Exemplary parasite and fungus and parasite and fungal disease
that may be treated or vaccinated by the cytoplast

| Target | Disease |
|---|---|
| VSG | Trypanosoma brucei |
| VSG | Human African Trypanosoma |
| Paraflagellar rod protein | Detection of all trypanosoma species |
| | (diagnostics) |
| Cell wall protein Malf1 | Malassezia furfur |
| Myosin tail interaction protein | Plasmodium falciparum |

TABLE 6
Exemplary toxin and toxin disease that may be treated
or vaccinated by the cytoplast

| Target | Disease |
|---|---|
| Toxic venom fractions: Aahl′ and | Androctonus australis hector |
| Aahll | (Aah) scorpion venom |
| HNc | Hemiscorpius lepturus |
| | scorpion venom |
| α-Cobratoxin | Naja kaouthia venom |
| RTA/RTB subunits | Ricin |
| CDTa toxin | Clostridium difficile |
| CDTa/CDTb toxin | C. difficile |
| LPS derived from | N. meningitidis |
| Neisseria meningitidis | |
| Staphylococcal enterotoxin B | Toxin of Cholera |
| Anthrax | Bacillus anthracis |
| Shiga toxin 1 and 2 | Shiga toxin-producing |
| | Escherichia coli |
| Botulinum neurotoxin A and E | Clostridium botulinum |
| ADP-ribosylating toxin | Salmonella typhimurium |
| Tetanus toxin and CD11b/CD18 | Clostridium tetani |
| (mac-1) | |

C. Active Agents

The cytoplasts of the present disclosure express or contain an active agent, such an anti-viral composition (e.g., vaccine, neutralizing antibodies against a pathogen). A active agent can comprise at least one of a therapeutic DNA molecule, a therapeutic RNA molecule, a therapeutic protein (e.g., an enzyme, an antibody, an antigen, a toxin, cytokine, a protein hormone, a growth factor, a cell surface receptor, or a vaccine), a therapeutic peptide (e.g., a peptide hormone or an antigen), a small molecule active agent (e.g., a steroid, a polyketide, an alkaloid, a toxin, an antibiotic, an antiviral, a colchicine, a taxol, a mitomycin, or emtansine), and a therapeutic gene editing factor. In some embodiments, a cytoplast can be engineered to produce (e.g., express, and in some embodiments, secrete) at least one of a therapeutic DNA molecule, a therapeutic RNA molecule, a therapeutic protein, a therapeutic peptide, a therapeutic small molecule, or a therapeutic gene editing component. Alternatively, or in addition, the nucleated cell (the “parent” cell as used herein) may be engineered to produce at least one of a therapeutic DNA molecule, a therapeutic RNA molecule, a therapeutic protein, a therapeutic peptide, a small molecule active agent, and a gene editing factor, prior to enucleation into a cytoplast.

The therapeutic DNA molecule, a therapeutic RNA molecule, a therapeutic protein, a therapeutic peptide, a small molecule active agent, or a therapeutic gene editing factor can include a targeting moiety. Non-limiting exemplary targeting moieties that can be produced by or contained in a cytoplast include chemokine receptors, adhesion molecules, and antigens.

A cytoplast of the present disclosure may be administered to a subject, and may contain a therapeutic DNA molecule, a therapeutic RNA molecule, a therapeutic protein (e.g., an enzyme, an antibody, an antigen, a toxin, cytokine, a protein hormone, a growth factor, a cell surface receptor, or a vaccine, or any therapeutic protein that is currently available or in development), a therapeutic peptide (e.g., a peptide hormone or an antigen, or any therapeutic peptide that is currently available or in development), a small molecule active agent (e.g., a steroid, a polyketide, an alkaloid, a toxin, an antibiotic, an antiviral, an analgesic, an anticoagulant, an antidepressant, an anticancer drug, an antiepileptic, an antipsychotic, a sedative, a colchicine, a taxol, a mitomycin, emtansine, or any small molecule active agent that is currently available or in development), a therapeutic gene editing factor, a therapeutic nanoparticle, or another active agent (e.g., bacteria, bacterial spores, bacteriophages, bacterial components, viruses (e.g., oncolytic viruses), exosomes, lipids, or ions). Non-limiting examples of oncolytic viruses include Talimogene laherparepvec, Onyx-015, GL-ONC1, CV706, Voyager-V1, and HSV-1716. Some wild-type viruses also show oncolytic behavior, such as Vaccinia virus, Vesicular stomatitis virus, Poliovirus, Reovirus, Senecavirus, ECHO-7, and Semliki Forest virus.

In some embodiments, the DNA molecule, the RNA molecule, the protein, the peptide, the small molecule active agent, and/or the gene-editing factor are recombinantly expressed. In some embodiments, the cell from which the cytoplast is derived or obtained is engineered to produce one or more of the DNA molecule, the RNA molecule, the protein, the peptide, the small molecule active agent, and/or the gene-editing factor. In some embodiments, the cell from which the cytoplast is derived or obtained is engineered to stably (e.g., permanently) express one or more of the DNA molecule, the RNA molecule, the protein, the peptide, the small molecule active agent, and/or the gene-editing factor. In some embodiments, the cell from which the cytoplast is derived or obtained is engineered to transiently express one or more of the DNA molecule, the RNA molecule, the protein, the peptide, the small molecule active agent, and/or the gene-editing factor. In some embodiments, the cell from which the cytoplast is derived or obtained is engineered prior to enucleation. In some embodiments, the cytoplast is engineered to transiently express one or more of the DNA molecule, the RNA molecule, the protein, the peptide, the small molecule active agent, and/or the gene-editing factor (e.g., engineered following enucleation).

In some embodiments, DNA molecule, the RNA molecule, the protein, the peptide, the small molecule active agent, and/or the gene-editing factor are not naturally expressed (e.g., in the absence of engineering) in the cell from which the cytoplast was derived or obtained (e.g., the DNA molecule, the RNA molecule, the protein, the peptide, the small molecule active agent, and/or the gene-editing factor are exogenous to the cytoplast). In some embodiments, the DNA molecule, the RNA molecule, the protein, the peptide, the small molecule active agent, and/or the gene-editing factor are not naturally expressed in the subject (e.g., the DNA molecule, the RNA molecule, the protein, the peptide, the small molecule active agent, and/or the gene-editing factor are exogenous to the subject). In some embodiments, the DNA molecule, the RNA molecule, the protein, the peptide, the small molecule active agent, and/or the gene-editing factor are not naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the DNA molecule, the RNA molecule, the protein, the peptide, the small molecule active agent, and/or the gene-editing factor are exogenous to the intended site of therapy).

In some embodiments, the DNA molecule, the RNA molecule, the protein, the peptide, the small molecule active agent, and/or the gene-editing factor are naturally expressed (e.g., in the absence of engineering) in the cell from which the cytoplast was derived or obtained (e.g., the DNA molecule, the RNA molecule, the protein, the peptide, the small molecule active agent, and/or the gene-editing factor are innately endogenous to the cytoplast) (e.g., in the absence of engineering of the cell from which the cytoplast was derived or obtained). In some embodiments, the DNA molecule, the RNA molecule, the protein, the peptide, the small molecule active agent, and/or the gene-editing factor are naturally expressed in the subject (e.g., the DNA molecule, the RNA molecule, the protein, the peptide, the small molecule active agent, and/or the gene-editing factor are endogenous to the subject). In some embodiments, the DNA molecule, the RNA molecule, the protein, the peptide, the small molecule active agent, and/or the gene-editing factor are naturally expressed in the subject at the intended site of therapy (e.g., a tumor, or a particular tissue, such as the brain, the intestine, the lungs, the heart, the liver, the spleen, the pancreas, muscles, eyes, and the like) (e.g., the DNA molecule, the RNA molecule, the protein, the peptide, the small molecule active agent, and/or the gene-editing factor are endogenous to the intended site of therapy).

In some embodiments, therapeutic, e.g., the DNA molecule, the RNA molecule, the protein, the peptide, the small molecule active agent, and/or the gene-editing factor, is derived from a synthetic cell and loaded into the cytoplast.

In some embodiments, the cytoplast expresses a corrected, a truncated, or a non-mutated version and/or copy of the DNA molecule, the RNA molecule, the protein, the peptide, the small molecule active agent, and/or the gene-editing factor as compared to the cell from which the cytoplast was derived or obtained. In some embodiments, the cytoplast is obtained from any nucleated cell (e.g., a eukaryotic cell, a mammalian cell (e.g., a human cell, or any mammalian cell described herein), a protozoal cell (e.g., an amoeba cell), an algal cell, a plant cell, a fungal cell, an invertebrate cell, a fish cell, an amphibian cell, a reptile cell, or a bird cell).

In some embodiments, a cytoplast produces or contains at least 2 (e.g., at least 2, 3, 4, 5, or more) different therapeutic DNA molecules, therapeutic RNA molecules, therapeutic proteins, therapeutic peptides, small molecule active agent s, or therapeutic gene-editing factors, in any combination. For example, in some embodiments, a cytoplast can produce or contain a therapeutic DNA molecule and a small molecule active agent. For example, in some embodiments, a cytoplast can produce or contain two different small molecule active agent s. For example, in some embodiments, a cytoplast can produce or contain a chemokine receptor (e.g., for targeting) and a small molecule active agent.

In some embodiments, the therapeutic RNA molecule is messenger RNA (mRNA), short hairpin RNA (shRNA), small interfering RNA (siRNA), microRNA, long non-coding RNA (lncRNA) or a RNA virus. In some embodiments, the therapeutic DNA molecule is single-stranded DNA, double-stranded DNA, an oligonucleotide, a plasmid, a bacterial DNA molecule or a DNA virus. In some embodiments, the therapeutic protein is a cytokine, a growth factor, a hormone, an antibody, a small-peptide based drug, or an enzyme. In some embodiments, the cytoplast transiently expresses the therapeutic DNA molecule, the therapeutic RNA molecule, the therapeutic protein, the therapeutic peptide, the small molecule therapeutic, and/or the therapeutic gene editing factor. In some embodiments, the expression of the therapeutic DNA molecule, the therapeutic RNA molecule, the therapeutic protein, the therapeutic peptide, the small molecule therapeutic, and/or the therapeutic gene editing factor is inducible. In some embodiments, a nucleated cell is permanently engineered to express the therapeutic DNA molecule, the therapeutic RNA molecule, the therapeutic protein, the therapeutic peptide, the small molecule therapeutic, and/or the therapeutic gene editing factor. In some embodiments, the expression of the therapeutic DNA molecule, the therapeutic RNA molecule, the therapeutic protein, the therapeutic peptide, the small molecule therapeutic, and/or the therapeutic gene editing factor. In some embodiments of any of the methods described herein, the cytoplast comprises a active agent or a nanoparticle. In some embodiments, the active agent is a small molecule or a bacteria or an exosome.

For the systemic administration of therapeutic cells, there are two major problems for their successful homing to the diseased tissues. First, most of the cells may be trapped in the small capillaries in the lung or other tissues, which may also cause serious side effects such as pulmonary embolism. Cytoplasts are, in some embodiments, much smaller than their parental cells (e.g., about 60% of the diameter of parental cells and ⅛ the volume) and do not have the rigid nucleus, therefore, cytoplasts can pass better through small capillaries and vessels than their parental cells. Second, the specific homing of cells to the diseased tissues can depend on the chemokine receptor signaling such as SDF-1α/CXCR4, CCL2/CCR2, and the adhesion molecules such as PSGL-1. As shown herein, cytoplasts can be engineered to specifically express functional CXCR4, CCR2 as well as glycosylated PSGL-1, which can greatly promote the specific homing of the engineered cytoplasts.

In some embodiments, the cytoplasts can further include (e.g. by engineering or from the cell from which they were obtained) a targeting moiety that is expressed on the cell surface of the cytoplast, e.g., CXCR4, CCR2 or PSGL-1. Non-limiting examples of cell surface proteins that may be expressed on the cell surface of the cytoplast include chemokines such as CXCR4, CCR2, CCR1, CCR5, CXCR7, CXCR2, and CXCR1. Other example of cell surface proteins that can be expressed on the cell surface of the cytoplast as homing receptor can include C-X-C chemokine receptor type 3, leukosialin, CD44 antigen, C-C chemokine receptor type 7, L-selectin, lymphocyte function-associated antigen 1, or very late antigen-4, or a combination thereof. In some embodiments, the cytoplasts can further include (e.g. by engineering or from the cell from which they were obtained) a cell targeting moiety that is secreted by the cytoplasts, or is tethered to the extracellular matrix, e.g., SDF1α or CCL2. Non-limiting examples of proteins that may be secreted by the cytoplast for cell homing include: SDF1α, CCL2, CCL3, CCL5, CCL8, CCL1, CXCL9, CXCL10, CCL11, and CXCL12. The targeting moiety may direct the cytoplast to a target cell, target tissue, or target environment. In some embodiments, the targeting moiety directs the cytoplast based on chemokine/chemokine receptor sensing. In some embodiments, the targeting moiety directs the cytoplast based on direct binding. For example, the targeting moiety may comprise an antibody that may bind to an antigen expressed by the target cell.

In some embodiments, the cytoplasts can express and/or secret at least one of cytokines selected from the group consisting of: 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD4OLG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-lb, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MEW class III, miscellaneous hematopoietins, monokine, MSP, myokine, myonectin, nicotinamide phosphoribosyltransferase, oncostatin M (OSM), oprelvekin, OX4OL, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2.

In some embodiments, the cytoplasts can express and/or secrete at least one cytokine to modulate biological activities of any one of myeloid cell, a T cell such as alpha beta cytotoxic T cell, a gamma delta T cell, a regulatory T cell, a natural killer T cell, a B cell, a natural killer cell, macrophages, mast cells, endothelial cells, fibroblasts, or various stromal cells.

In some embodiments, the cytoplasts can further include (e.g. by engineering or from the cell from which they were obtained) a surface marker that aids in their evasion of the subject immune system. For example, in some embodiments, the cytoplasts can include a CD47 marker. Without being bound by any particular theory, it is believed that a CD47 marker helps to prevent the cytoplasts from being phagocytosed by macrophages. Non-limiting examples of cell-matrix receptors and cell-cell adhesion molecules include integrins, cadherins, glycoproteins, and heparin sulfate proteoglycans. Non-limiting examples of therapeutic molecules include tumor antigens and immunomodulatory peptides, polyamines, and ATP.

1. Vaccine Compositions

Described herein, in some embodiments, are cytoplasts engineered to express or deliver an active agent that is a vaccine composition. In some embodiments, a nucleic acid molecule encoding the vaccine composition is introduced into the cytoplast, or parent cell thereof, using the methods described herein. In some embodiments, the vaccine composition is expressed in the cytoplast using cell machinery endogenous to the corresponding parent cell (e.g., mRNA translational machinery, protein synthesis). Once administered to the subject, in some embodiments, the cytoplast utilizes endogenous protein secretion machinery of the corresponding parent cell to secrete the vaccine composition into extracellular space. The cytoplasts may also be engineered with homing receptors specific to target tissues in the subject (e.g., lung, lymph) in which the vaccine composition is secreted. The cytoplasts may also be engineered to express immune system activators, such as granulocyte-macrophage colony-stimulating factor (GM-CSF) or any one of the cytokines or receptors for the cytokines described herein.

In some embodiments, the vaccine composition is against an antigen of a pathogen. Non-limiting examples of antigens include proteins comprising native sequences, polypeptides comprising natural or unnatural amino acids and/or with modifications such as glycosylation, palmitoylation, myristoylation, and the like, and nucleic acids comprising natural or unnatural bases. A pathogen can be any bacteria, virus, or fungus that causes infection in a mammal. In some embodiments, a pathogen can be a virus. In some embodiments, the viral antigen can be prepared from a viral protein, a fragment of a viral protein, or nucleic acid encoding the viral protein or the fragment of the viral protein. In some embodiments, the vaccine comprises an inactivated version of a virus described herein. In some embodiments, the vaccine comprises a live-attenuated version of a virus described herein. A live-attenuated virus, in some embodiments, is a virus that is alive but is replication deficient. A live-attenuated virus, in other cases, is a virus that is alive but is not infectious.

In some embodiments, the vaccine comprising the cytoplast described herein induces an adaptive immune response in the subject following administered of the cytoplast comprising the vaccine composition to a subject. In some embodiments, the vaccine described herein induce an adaptive immune response that is sufficient to immunize the subject against an infection by the virus, or lessen the severity of a disease or condition caused by an infection by the virus.

Provided herein, in some embodiments, are cytoplasts engineered to express a vaccine compositions against a viral antigen of a pathogen disclosed herein. The virus can be a DNA virus or an RNA virus. A DNA virus can be a single-stranded (ss) DNA virus, a double-stranded (ds) DNA virus, or a DNA virus that contains both ss and ds DNA regions. An RNA virus can be a single-stranded (ss) RNA virus or a double-stranded (ds) RNA virus. A ssRNA virus can further be classified into a positive-sense RNA virus or a negative-sense RNA virus.

In some embodiments, the viral antigen is at least or equal to 50%, 60%, 70%, 75%, 80%, 85%, 90%, 95%, or 99% identical to an influenza protein encoded by any, genera, strain, or subtype of influenza. Exemplary influenza genus can include Influenza virus A, Influenza virus B, Influenza virus C, and Influenza virus D. In some embodiments, the cytoplast described herein can be engineered to express a combination of influenza viral proteins of hemagglutinin (HA) and neuraminidase (NA). Influenza hemagglutinin (HA) that can be expressed by the cytoplast described herein can include HA subtype H1, H2, H3, H4, H5, H6, H7, H8, H9, H10, H11, H12, H13, H14, H15, H16, H17, or H18. Influenza neuraminidase (NA) that can be expressed by the cytoplast described herein can include NA subtype N1, N2, N3, N4, N5, N6, N7, N8, N9, N10, or N11. In some embodiments, the cytoplast described herein can express a combination of any one of the HA and NA subtype described herein. Exemplary combination that can be expressed by one a single cytoplast can include H1N1, H2N2, H3N2, H5N1, H7N7, H1N2, H9N2, H7N2, H7N3, H10N7, H7N9, or H6N1. Other additional exemplary combination can include H1N1, H1N2, H1N3, H1N4, H1N5, H1NG, H1N7, H1N8, H1N9, H1N10, H1N11, H2N1, H2N2, H2N3, H2N4, H2N5, H2NG, H2N7, H2N8, H2NB, H2N1D, H2N11, H3N1, H3N2, H3N3, H3N4, H3N5, H3NB, H3N7, H3N8, H3NB, H3N1D, H3N11, H4N1, H4N2, H4N3, H4N4, H4N5, H4NB, H4N7, H4N8, H4N9, H4N10, H4N11, H5N1, H5N2, H5N3, H5N4, H5N5, H5NB, H5N7, H5N8, H5N3, H5N1D, H5N11, HBN1, HBN2, HBN3, HBN4, HBN5, HBNB, HBN7, HBN8, HBN9, HBN10, HBN11, H7N1, H7N2, H7N3, H7N4, H7N5, H7NB, H7N7, H7N8, H7N9, H7N10, H7N11, H8N1, H8N2, H8N3, H8N4, H8N5, H8NG, H8N7, H8N8,5 H8N9, H8N10, HBN11, HBN1, H9N2, HBN3, H9N4, H3N5, H3N7, H3N8, H3N3, H9N1D, HBN11, H1DN1, H10N2, H1DN3, H1DN4, H1DN5, H1DNG, H1DN7, H1DN8, H1DN3, H10N10, H1DN11, H11N1, H11N2, H11N3, H11N4, H11N5, HUNG, H11N7, HUNS, H11NS, H11N10, H11N11, H12N1, H12N2, H12N3, H12N4, H12N5, H12NB, H12N7, H12N8, H12N3, H12N1D, H12N11, H13N1, H13N2, H13N3, H13N4, H13N5, H13NB, H13N7, H13N8, H13N3, H13N1D, H13N11, H14N1, H14N2, H14N3, H14N4, H14N5, H14NB, H14N7, H14N8, H14N9, H14N10, H14N11, H15N1, H15N2, H15N3, H15N4, H15N5, H15NB, H15N7, H15N8, H15N3, H15N1D, H15N11, H1BN1, H1BN2, H1BN3, H1BN4, H1BN5, H1BNB, H1BN7, H1BN8, H1GN3, H1BN10, H1BN11, H17N1, H17N2, H17N3, H17N4, H17N5, H17NB, H17N7, H17N8, H17N3, H17N10, H17N11, H1BN1, H18N2, H18N3, H18N4, H18N5, H1BNB, H18N7, H18N8, H18N3, H1BN10, or H1BN11.

Provided herein, in some embodiments, are cytoplasts engineered to express a vaccine composition against a bacterial antigen. In some embodiments, the bacterial antigen is derived from anthrax (Bacillus anthracis), Botulism (Clostridium botulinum toxin), plague (Yersinia pestis), tularemia (Francisella tularensis), Brucellosis (Brucella species), epsilon toxin of Clostridium perfringens, salmonella species, Escherichia coli O 157:H7, Shigella, Glanders (Burkholderia mallei), Melioidosis (Burkholderia pseudomallei), Psittacosis (Chlamydia psittaci), Q fever (Coxiella burnetii), Staphylococcal enterotoxin B, Typhus fever (Rickettsia prowazekii) Vibrio cholerae, Cryptosporidium parvum. In some embodiments, the cytoplasts are engineered to express a vaccine composition against Ricin toxin from Ricinus communis (castor beans).

Provided herein, in some embodiments, are cytoplasts engineered to express a vaccine compositions against a tumor antigen. A “tumor antigen” as used herein refers to an antigen produced by a cancer cell. Non-limiting examples of cancer cell or tumor cell, as used in the present disclosure, can include cell of cancer including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia,Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. In some embodiments, the cancer can be lung cancer, including non-small cell lung cancer (NSCLC), small cell lung cancer (SCLC), or any other lung cancer type. For example, the lung cancer can include adenocarcinoma, squamous carcinoma, large cell (undifferentiated) carcinoma, large cell neuroendocrine carcinoma, adenosquamous carcinoma, sarcomatoid carcinoma, lung carcinoid tumor, or adenoid cystic carcinoma. Other exemplary lung cancer can include lymphoma, sarcoma, benign lung tumor, or hamartoma.

a. Antigens

Described herein, in some embodiments, is a cytoplast comprising at least one antigen, or portion thereof, expressed by the cytoplast. In some embodiments, the at least one antigen may be an antigen expressed or released by a cancer cell. In some embodiments, the at least one antigen may be an antigen expressed or released by a pathogen. In some embodiments, the at least one antigen may be an antigen expressed or released by a virus. In some embodiments, the at least one antigen may be an antigen expressed or released by a bacterium. In some embodiments, the at least one antigen may be an antigen expressed or released by a fungus. In some embodiments, the at least one antigen can be encoded by at least one heterologous polynucleotide, where the at least one heterologous polynucleotide can be a cargo of the cytoplast. In some embodiments, the heterologous polynucleotide can comprise a viral vector or a plasmid. In some embodiments, the cytoplast delivers the heterologous polynucleotide to the target tissue. In some embodiments, the cytoplast comprising the at least one antigen or comprising the heterologous polynucleotide encoding the at least one antigen can be part of the vaccine described in the instant specification.

In some embodiments, the at least one antigen, or portion thereof, may be a cancer antigen expressed or associated with a cancer cell. In some embodiments, the cytoplast expresses at least one cancer antigen on the surface of the cytoplast. In some embodiments, the cytoplast releases or secretes at least one cancer antigen. In some embodiments, the at least one cancer antigen may be a cargo of the cytoplast. In some embodiments, the cytoplast delivers the at least one cancer antigen to target cell or tissue. The cancer antigen may be expressed by any one of the cancer cell described herein. In some embodiments, the cancer antigen expressed or released by the cytoplast described herein may be sufficient to trigger immune response (e.g. B cell activation), when the cytoplast is administered to a subject.

In some embodiments, the cytoplast comprises at least one cancer antigen, or a portion thereof. In some embodiments, the cytoplast comprise one, two, three, four, five, six, seven, eight, nine, ten, 20, 50, 100, 200, 500, 1000, 2000, 5000, 10000, or more cancer antigens. In some embodiments, the cancer antigen is at least or equal to 50%, 60%, 70%, 80%, 90%, 95%, or 99% identical to a peptidyl sequence of an antigen expressed or associated with a cancer cell.

In some embodiments, the cytoplast comprise one, two, three, four, five, six, seven, eight, nine, ten, 20, 50, 100, 200, 500, 1000, 2000, 5000, 10000, or more antigens. In some embodiments, the antigen is greater than or equal to about 50%, 60%, 70%, 80%, 90%, 95%, or 99% identical to a peptidyl sequence of an antigen described herein. In some embodiments, the antigen or portion thereof, comprises an amino acid length between about 5 amino acids to about 5,000 amino acids. In some embodiments, the antigen or portion thereof comprises an amino acid length between about 5 amino acids to about 10 amino acids, about 5 amino acids to about 15 amino acids, about 5 amino acids to about 20 amino acids, about 5 amino acids to about 25 amino acids, about 5 amino acids to about 50 amino acids, about 5 amino acids to about 100 amino acids, about 5 amino acids to about 200 amino acids, about 5 amino acids to about 500 amino acids, about 5 amino acids to about 1,000 amino acids, about 5 amino acids to about 2,000 amino acids, about 5 amino acids to about 5,000 amino acids, about 10 amino acids to about 15 amino acids, about 10 amino acids to about 20 amino acids, about 10 amino acids to about 25 amino acids, about 10 amino acids to about 50 amino acids, about 10 amino acids to about 100 amino acids, about 10 amino acids to about 200 amino acids, about 10 amino acids to about 500 amino acids, about 10 amino acids to about 1,000 amino acids, about 10 amino acids to about 2,000 amino acids, about 10 amino acids to about 5,000 amino acids, about 15 amino acids to about 20 amino acids, about 15 amino acids to about 25 amino acids, about 15 amino acids to about 50 amino acids, about 15 amino acids to about 100 amino acids, about 15 amino acids to about 200 amino acids, about 15 amino acids to about 500 amino acids, about 15 amino acids to about 1,000 amino acids, about 15 amino acids to about 2,000 amino acids, about 15 amino acids to about 5,000 amino acids, about 20 amino acids to about 25 amino acids, about 20 amino acids to about 50 amino acids, about 20 amino acids to about 100 amino acids, about 20 amino acids to about 200 amino acids, about 20 amino acids to about 500 amino acids, about 20 amino acids to about 1,000 amino acids, about 20 amino acids to about 2,000 amino acids, about 20 amino acids to about 5,000 amino acids, about 25 amino acids to about 50 amino acids, about 25 amino acids to about 100 amino acids, about 25 amino acids to about 200 amino acids, about 25 amino acids to about 500 amino acids, about 25 amino acids to about 1,000 amino acids, about 25 amino acids to about 2,000 amino acids, about 25 amino acids to about 5,000 amino acids, about 50 amino acids to about 100 amino acids, about 50 amino acids to about 200 amino acids, about 50 amino acids to about 500 amino acids, about 50 amino acids to about 1,000 amino acids, about 50 amino acids to about 2,000 amino acids, about 50 amino acids to about 5,000 amino acids, about 100 amino acids to about 200 amino acids, about 100 amino acids to about 500 amino acids, about 100 amino acids to about 1,000 amino acids, about 100 amino acids to about 2,000 amino acids, about 100 amino acids to about 5,000 amino acids, about 200 amino acids to about 500 amino acids, about 200 amino acids to about 1,000 amino acids, about 200 amino acids to about 2,000 amino acids, about 200 amino acids to about 5,000 amino acids, about 500 amino acids to about 1,000 amino acids, about 500 amino acids to about 2,000 amino acids, about 500 amino acids to about 5,000 amino acids, about 1,000 amino acids to about 2,000 amino acids, about 1,000 amino acids to about 5,000 amino acids, or about 2,000 amino acids to about 5,000 amino acids. In some embodiments, the cancer antigen comprises an amino acid length between about 5 amino acids, about 10 amino acids, about 15 amino acids, about 20 amino acids, about 25 amino acids, about 50 amino acids, about 100 amino acids, about 200 amino acids, about 500 amino acids, about 1,000 amino acids, about 2,000 amino acids, or about 5,000 amino acids. In some embodiments, the cancer antigen comprises an amino acid length between at least about 5 amino acids, about 10 amino acids, about 15 amino acids, about 20 amino acids, about 25 amino acids, about 50 amino acids, about 100 amino acids, about 200 amino acids, about 500 amino acids, about 1,000 amino acids, or about 2,000 amino acids. In some embodiments, the cancer antigen comprises an amino acid length between at most about 10 amino acids, about 15 amino acids, about 20 amino acids, about 25 amino acids, about 50 amino acids, about 100 amino acids, about 200 amino acids, about 500 amino acids, about 1,000 amino acids, about 2,000 amino acids, or about 5,000 amino acids. In some embodiments, the cancer antigen comprises an amino acid length at least about 5 amino acids to about 5,000 amino acids. In some embodiments, the cancer antigen comprises an amino acid length at least about 5 amino acids to about 10 amino acids, about 5 amino acids to about 15 amino acids, about 5 amino acids to about 20 amino acids, about 5 amino acids to about 25 amino acids, about 5 amino acids to about 50 amino acids, about 5 amino acids to about 100 amino acids, about 5 amino acids to about 200 amino acids, about 5 amino acids to about 500 amino acids, about 5 amino acids to about 1,000 amino acids, about 5 amino acids to about 2,000 amino acids, about 5 amino acids to about 5,000 amino acids, about 10 amino acids to about 15 amino acids, about 10 amino acids to about 20 amino acids, about 10 amino acids to about 25 amino acids, about 10 amino acids to about 50 amino acids, about 10 amino acids to about 100 amino acids, about 10 amino acids to about 200 amino acids, about 10 amino acids to about 500 amino acids, about 10 amino acids to about 1,000 amino acids, about 10 amino acids to about 2,000 amino acids, about 10 amino acids to about 5,000 amino acids, about 15 amino acids to about 20 amino acids, about 15 amino acids to about 25 amino acids, about 15 amino acids to about 50 amino acids, about 15 amino acids to about 100 amino acids, about 15 amino acids to about 200 amino acids, about 15 amino acids to about 500 amino acids, about 15 amino acids to about 1,000 amino acids, about 15 amino acids to about 2,000 amino acids, about 15 amino acids to about 5,000 amino acids, about 20 amino acids to about 25 amino acids, about 20 amino acids to about 50 amino acids, about 20 amino acids to about 100 amino acids, about 20 amino acids to about 200 amino acids, about 20 amino acids to about 500 amino acids, about 20 amino acids to about 1,000 amino acids, about 20 amino acids to about 2,000 amino acids, about 20 amino acids to about 5,000 amino acids, about 25 amino acids to about 50 amino acids, about 25 amino acids to about 100 amino acids, about 25 amino acids to about 200 amino acids, about 25 amino acids to about 500 amino acids, about 25 amino acids to about 1,000 amino acids, about 25 amino acids to about 2,000 amino acids, about 25 amino acids to about 5,000 amino acids, about 50 amino acids to about 100 amino acids, about 50 amino acids to about 200 amino acids, about 50 amino acids to about 500 amino acids, about 50 amino acids to about 1,000 amino acids, about 50 amino acids to about 2,000 amino acids, about 50 amino acids to about 5,000 amino acids, about 100 amino acids to about 200 amino acids, about 100 amino acids to about 500 amino acids, about 100 amino acids to about 1,000 amino acids, about 100 amino acids to about 2,000 amino acids, about 100 amino acids to about 5,000 amino acids, about 200 amino acids to about 500 amino acids, about 200 amino acids to about 1,000 amino acids, about 200 amino acids to about 2,000 amino acids, about 200 amino acids to about 5,000 amino acids, about 500 amino acids to about 1,000 amino acids, about 500 amino acids to about 2,000 amino acids, about 500 amino acids to about 5,000 amino acids, about 1,000 amino acids to about 2,000 amino acids, about 1,000 amino acids to about 5,000 amino acids, or about 2,000 amino acids to about 5,000 amino acids. In some embodiments, the antigen or portion thereof comprises an amino acid length at least about 5 amino acids, about 10 amino acids, about 15 amino acids, about 20 amino acids, about 25 amino acids, about 50 amino acids, about 100 amino acids, about 200 amino acids, about 500 amino acids, about 1,000 amino acids, about 2,000 amino acids, or about 5,000 amino acids. In some embodiments, the cancer antigen comprises an amino acid length at least at least about 5 amino acids, about 10 amino acids, about 15 amino acids, about 20 amino acids, about 25 amino acids, about 50 amino acids, about 100 amino acids, about 200 amino acids, about 500 amino acids, about 1,000 amino acids, or about 2,000 amino acids. In some embodiments, the cancer antigen comprises an amino acid length at least at most about 10 amino acids, about 15 amino acids, about 20 amino acids, about 25 amino acids, about 50 amino acids, about 100 amino acids, about 200 amino acids, about 500 amino acids, about 1,000 amino acids, about 2,000 amino acids, or about 5,000 amino acids. In some embodiments, the antigen or portion thereof comprises an amino acid length at most about 5 amino acids to about 5,000 amino acids. In some embodiments, the cancer antigen comprises an amino acid length at most about 5 amino acids to about 10 amino acids, about 5 amino acids to about 15 amino acids, about 5 amino acids to about 20 amino acids, about 5 amino acids to about 25 amino acids, about 5 amino acids to about 50 amino acids, about 5 amino acids to about 100 amino acids, about 5 amino acids to about 200 amino acids, about 5 amino acids to about 500 amino acids, about 5 amino acids to about 1,000 amino acids, about 5 amino acids to about 2,000 amino acids, about 5 amino acids to about 5,000 amino acids, about 10 amino acids to about 15 amino acids, about 10 amino acids to about 20 amino acids, about 10 amino acids to about 25 amino acids, about 10 amino acids to about 50 amino acids, about 10 amino acids to about 100 amino acids, about 10 amino acids to about 200 amino acids, about 10 amino acids to about 500 amino acids, about 10 amino acids to about 1,000 amino acids, about 10 amino acids to about 2,000 amino acids, about 10 amino acids to about 5,000 amino acids, about 15 amino acids to about 20 amino acids, about 15 amino acids to about 25 amino acids, about 15 amino acids to about 50 amino acids, about 15 amino acids to about 100 amino acids, about 15 amino acids to about 200 amino acids, about 15 amino acids to about 500 amino acids, about 15 amino acids to about 1,000 amino acids, about 15 amino acids to about 2,000 amino acids, about 15 amino acids to about 5,000 amino acids, about 20 amino acids to about 25 amino acids, about 20 amino acids to about 50 amino acids, about 20 amino acids to about 100 amino acids, about 20 amino acids to about 200 amino acids, about 20 amino acids to about 500 amino acids, about 20 amino acids to about 1,000 amino acids, about 20 amino acids to about 2,000 amino acids, about 20 amino acids to about 5,000 amino acids, about 25 amino acids to about 50 amino acids, about 25 amino acids to about 100 amino acids, about 25 amino acids to about 200 amino acids, about 25 amino acids to about 500 amino acids, about 25 amino acids to about 1,000 amino acids, about 25 amino acids to about 2,000 amino acids, about 25 amino acids to about 5,000 amino acids, about 50 amino acids to about 100 amino acids, about 50 amino acids to about 200 amino acids, about 50 amino acids to about 500 amino acids, about 50 amino acids to about 1,000 amino acids, about 50 amino acids to about 2,000 amino acids, about 50 amino acids to about 5,000 amino acids, about 100 amino acids to about 200 amino acids, about 100 amino acids to about 500 amino acids, about 100 amino acids to about 1,000 amino acids, about 100 amino acids to about 2,000 amino acids, about 100 amino acids to about 5,000 amino acids, about 200 amino acids to about 500 amino acids, about 200 amino acids to about 1,000 amino acids, about 200 amino acids to about 2,000 amino acids, about 200 amino acids to about 5,000 amino acids, about 500 amino acids to about 1,000 amino acids, about 500 amino acids to about 2,000 amino acids, about 500 amino acids to about 5,000 amino acids, about 1,000 amino acids to about 2,000 amino acids, about 1,000 amino acids to about 5,000 amino acids, or about 2,000 amino acids to about 5,000 amino acids. In some embodiments, the cancer antigen comprises an amino acid length at most about 5 amino acids, about 10 amino acids, about 15 amino acids, about 20 amino acids, about 25 amino acids, about 50 amino acids, about 100 amino acids, about 200 amino acids, about 500 amino acids, about 1,000 amino acids, about 2,000 amino acids, or about 5,000 amino acids. In some embodiments, the cancer antigen comprises an amino acid length at most at least about 5 amino acids, about 10 amino acids, about 15 amino acids, about 20 amino acids, about 25 amino acids, about 50 amino acids, about 100 amino acids, about 200 amino acids, about 500 amino acids, about 1,000 amino acids, or about 2,000 amino acids. In some embodiments, the cancer antigen comprises an amino acid length at most at most about 10 amino acids, about 15 amino acids, about 20 amino acids, about 25 amino acids, about 50 amino acids, about 100 amino acids, about 200 amino acids, about 500 amino acids, about 1,000 amino acids, about 2,000 amino acids, or about 5,000 amino acids.

In some embodiments, the cytoplast expresses the antigen on the surface of the cytoplast. In some embodiments, the cytoplast releases or secretes the antigen. In some embodiments, the antigen may be a cargo of the cytoplast. In some embodiments, the cytoplast delivers the antigen to target cell or tissue. In some embodiments, the antigen expressed or released by the cytoplast described herein may be sufficient to trigger immune response (e.g. B cell activation), when the cytoplast is administered to a subject.

In some embodiments, the antigen or portion thereof, is a cancer antigen. In some embodiments, the cancer antigen is a pathogen antigen that is introduced into a cancer cell. For example, the cytoplast can be engineered to introduce a Spike protein of the SARS-CoV-2 virus into the cancer cell. In such scenario, a subject who has been vaccinated against SARS-CoV-2 would have acquired adaptive immune system that can target and kill the cancer cell. In some embodiments, the cancer antigen can be introduced into cancer cell by utilizing oncolytic virus as a vector (loaded into the cytoplast) to introduce the mRNA into the cancer cell.

In some embodiments, the at least one antigen may be a pathogen antigen. In some embodiments, the pathogen antigen is a viral antigen, a bacterial antigen, a fungal antigen, or a toxin antigen. The antigen may be expressed by any one of the described herein (e.g., any one of the pathogens in Table 3-6). In some embodiments, the at least one antigen may be a viral antigen. The viral antigen may be an antigen of a virus described herein (e.g., SARS-CoV-2). In some embodiments, the antigen is derived from a coronavirus. In some embodiments, the cytoplast comprises at least one viral antigen that is Spike protein (S protein) or a fragment of the Spike protein of the coronavirus. In some embodiments, the Spike protein or a fragment thereof can be a monomer or a trimer. In some embodiments, the Spike protein is a prefusion stabilized Spike protein. In some embodiments, the coronavirus is SARS-CoV-2.

In some embodiments, the viral antigen of the Spike protein or a fragment thereof is at least or equal to 50%, 60%, 70%, 80%, 90%, 95%, or 99% identical to SEQ ID NOs: 2 or 8. In some embodiments, the viral antigen comprising the Spike protein or a fragment thereof comprises at least one mutation or variant as described in da Silva Filipe, A., Shepherd, J. G., Williams, T. et al. Genomic epidemiology reveals multiple introductions of SARS-CoV-2 from mainland Europe into Scotland. Nat Microbiol 6, 112-122 (2021), the entirety of which is incorporated herein. In some embodiments, the viral antigen comprising the Spike protein or a fragment thereof comprises at least one mutation comprising Asp614Gly, with reference to SEQ ID NO: 2.

In some embodiments, the viral antigen of the Spike protein or a fragment thereof comprise an amino acid length at least or equal to 5 amino acids, 10 amino acids, 20 amino acids, 25 amino acids, 50 amino acids, 100 amino acids, 200 amino acids, or more. In some embodiments, the Spike protein or a fragment thereof is expressed on the surface of the cytoplast. In some embodiments, the Spike protein or a fragment thereof is secreted by the cytoplast. In some embodiments, the Spike protein or a fragment thereof is a cargo of the cytoplast. In some embodiments, the Spike protein or a fragment thereof is delivered by the cytoplast to target tissue. In some embodiments, the cytoplast comprising the Spike protein of a fragment thereof can induce an immune response in the subject. In some embodiments, the cytoplast comprising the Spike protein of a fragment thereof can induce and confer an adaptive immunity to SARS-CoV-2 infection. In some embodiments, the cytoplast comprising the Spike protein of a fragment thereof can treat or prevent SARS-CoV-2 infection. In some embodiments, the cytoplast expresses the Spike protein on the surface of the cytoplast. In some embodiments, the cytoplast secretes the Spike protein. In some embodiments, the cytoplast delivers the Spike protein to target tissue. In some embodiments, the cytoplast expresses the Spike protein on the surface of the cytoplast, secretes the Spike protein and/or delivers the Spike protein to target tissue.

In some embodiments, the cytoplast comprises at least one viral antigen that is Nucleocapsid protein (N protein) or a fragment of the n protein. In some embodiments, the viral antigen of the Nucleocapsid protein or a fragment thereof is at least or equal to 50%, 60%, 70%, 80%, 90%, 95%, or 99% identical to SEQ ID NO: 9. In some embodiments, the viral antigen of the Nucleocapsid protein or a fragment thereof comprise an amino acid length at least or equal to 5 amino acids, 10 amino acids, 20 amino acids, 25 amino acids, 50 amino acids, 100 amino acids, 200 amino acids, or more. In some embodiments, the Nucleocapsid protein or a fragment thereof is expressed on the surface of the cytoplast. In some embodiments, the Nucleocapsid protein or a fragment thereof is secreted by the cytoplast. In some embodiments, the Nucleocapsid protein or a fragment thereof is a cargo of the cytoplast. In some embodiments, the Nucleocapsid protein or a fragment thereof is delivered by the cytoplast to target tissue. In some embodiments, the cytoplast comprising the Nucleocapsid protein of a fragment thereof can induce an immune response in the subject. In some embodiments, the cytoplast comprising the Nucleocapsid protein of a fragment thereof can induce and confer an adaptive immunity to SARS-CoV-2 infection. In some embodiments, the cytoplast comprising the Nucleocapsid protein of a fragment thereof can treat or prevent SARS-CoV-2 infection. In some embodiments, the cytoplast expresses the Nucleocapsid protein on the surface of the cytoplast. In some embodiments, the cytoplast secretes the Nucleocapsid protein. In some embodiments, the cytoplast delivers the Nucleocapsid protein to target tissue. In some embodiments, the cytoplast expresses the Nucleocapsid protein on the surface of the cytoplast, secretes the Nucleocapsid protein and/or delivers the Nucleocapsid protein to target tissue.

In some embodiments, the cytoplast comprises at least one viral antigen that is Membrane protein (M protein) or a fragment of the n protein. In some embodiments, the viral antigen of the Membrane protein or a fragment thereof is at least or equal to 50%, 60%, 70%, 80%, 90%, 95%, or 99% identical to SEQ ID NO: 10. In some embodiments, the viral antigen of the Membrane protein or a fragment thereof comprise an amino acid length at least or equal to 5 amino acids, 10 amino acids, 20 amino acids, 25 amino acids, 50 amino acids, 100 amino acids, 200 amino acids, or more. In some embodiments, the Membrane protein or a fragment thereof is expressed on the surface of the cytoplast. In some embodiments, the Membrane protein or a fragment thereof is secreted by the cytoplast. In some embodiments, the Membrane protein or a fragment thereof is a cargo of the cytoplast. In some embodiments, the Membrane protein or a fragment thereof is delivered by the cytoplast to target tissue. In some embodiments, the cytoplast comprising the Membrane protein of a fragment thereof can induce an immune response in the subject. In some embodiments, the cytoplast comprising the Membrane protein of a fragment thereof can induce and confer an adaptive immunity to SARS-CoV-2 infection. In some embodiments, the cytoplast comprising the Membrane protein of a fragment thereof can treat or prevent SARS-CoV-2 infection. In some embodiments, the cytoplast expresses the Membrane protein on the surface of the cytoplast. In some embodiments, the cytoplast secretes the Membrane protein. In some embodiments, the cytoplast delivers the Membrane protein to target tissue. In some embodiments, the cytoplast expresses the Membrane protein on the surface of the cytoplast, secretes the Membrane protein and/or delivers the Membrane protein to target tissue.

In some embodiments, the cytoplast comprises at least one viral antigen that is Envelope protein (E protein) or a fragment of the n protein. In some embodiments, the viral antigen of the Envelope protein or a fragment thereof is at least or equal to 50%, 60%, 70%, 80%, 90%, 95%, or 99% identical to SEQ ID NO: 11. In some embodiments, the viral antigen of the Envelope protein or a fragment thereof comprise an amino acid length at least or equal to 5 amino acids, 10 amino acids, 20 amino acids, 25 amino acids, 50 amino acids, 100 amino acids, 200 amino acids, or more. In some embodiments, the Envelope protein or a fragment thereof is expressed on the surface of the cytoplast. In some embodiments, the Envelope protein or a fragment thereof is secreted by the cytoplast. In some embodiments, the Envelope protein or a fragment thereof is a cargo of the cytoplast. In some embodiments, the Envelope protein or a fragment thereof is delivered by the cytoplast to target tissue. In some embodiments, the cytoplast comprising the Envelope protein of a fragment thereof can induce an immune response in the subject. In some embodiments, the cytoplast comprising the Envelope protein of a fragment thereof can induce and confer an adaptive immunity to SARS-CoV-2 infection. In some embodiments, the cytoplast comprising the Envelope protein of a fragment thereof can treat or prevent SARS-CoV-2 infection. In some embodiments, the cytoplast expresses the Envelope protein on the surface of the cytoplast. In some embodiments, the cytoplast secretes the Envelope protein. In some embodiments, the cytoplast delivers the Envelope protein to target tissue. In some embodiments, the cytoplast expresses the Envelope protein on the surface of the cytoplast, secretes the Envelope protein and/or delivers the Envelope protein to target tissue.

In some embodiments, the viral antigen is encoded by a nucleic acid sequence that is at least or equal to 50%, 60%, 70%, 80%, 90%, 95%, or 99% identical to a fragment of any one of SEQ ID NOs: 4-7. In some embodiments, the cytoplast comprises at least one viral antigen encoded by a nucleic acid sequence that is 100% identical to a fragment of any one of SEQ ID NOs: 4-7.

In some embodiments, the viral antigen is derived from a coronavirus variant. In some embodiments, In some embodiments, the coronavirus variant antigen comprises an amino acid sequence that is at least or equal to about 50%, 60%, 70%, 75%, 80%, 85%, 90%, 95%, or 99% identical to one or more of SEQ ID NOs: 401-447 or 551-562. In some embodiments, the coronavirus variant antigen is encoded from a nucleic acid sequence that is at least or equal to about 50%, 60%, 70%, 75%, 80%, 85%, 90%, 95%, or 99% identical to one or more of SEQ ID NOs: 301-347 or 501-512. In some embodiments, administration of the cytoplast expressing antigen derived from the coronavirus variant to a subject is therapeutically effective to confer immunity against an infection by the coronavirus variant, or reduce disease severity caused by the coronavirus variant, in the subject.

In some embodiments, the viral antigen is derived from an avian coronavirus. In some embodiments, In some embodiments, the avian coronavirus antigen comprises an amino acid sequence that is at least or equal to about 50%, 60%, 70%, 75%, 80%, 85%, 90%, 95%, or 99% identical to one or more of SEQ ID NOs: 251-260. In some embodiments, the avian coronavirus antigen is encoded from a nucleic acid sequence that is at least or equal to about 50%, 60%, 70%, 75%, 80%, 85%, 90%, 95%, or 99% identical to one or more of SEQ ID NOs: 201-209. In some embodiments, administration of the cytoplast expressing antigen derived from the avian coronavirus to a subject is therapeutically effective to confer immunity against an infection by the avian coronavirus, or reduce disease severity caused by the avian coronavirus, in the subject.

In some embodiments, the antigen is derived from an ebolavirus. In some embodiments, the antigen is at least or equal to about 50%, 60%, 70%, 75%, 80%, 85%, 90%, 95%, or 99% identical to ebolavirus glycoprotein, matrix protein, nucleoprotein, nucleocapsid protein (e.g., VP30, VP35, or VP24), or polymerase (L) protein. In some embodiments, the antigen comprises an amino acid sequence that is at least or equal to about 50%, 60%, 70%, 75%, 80%, 85%, 90%, 95%, or 99% identical to one or more of SEQ ID NOs: 851-859. In some embodiments, the antigen is encoded from a nucleic acid sequence that is at least or equal to about 50%, 60%, 70%, 75%, 80%, 85%, 90%, 95%, or 99% identical to one or more of SEQ ID NOs: 801-809. In some embodiments, administration of the cytoplast expressing antigen derived from the ebolavirus to a subject is therapeutically effective to confer immunity against an infection by the ebolavirus, or reduce disease severity caused by the ebolavirus, in the subject.

In some embodiments, the viral antigen is derived from a hantavirus. In some embodiments, In some embodiments, the antigen is at least or equal to about 50%, 60%, 70%, 75%, 80%, 85%, 90%, 95%, or 99% identical to hantaviral polymerase, the M segment encodes the precursor (GPC) for two viral surface glycoproteins (Gn and Gc), and the S segment encodes the nucleocapsid (N) protein. In some embodiments, the antigen comprises an amino acid sequence that is at least or equal to about 50%, 60%, 70%, 75%, 80%, 85%, 90%, 95%, or 99% identical to one or more of SEQ ID NOs: 151-154. In some embodiments, the antigen is encoded from a nucleic acid sequence that is at least or equal to about 50%, 60%, 70%, 75%, 80%, 85%, 90%, 95%, or 99% identical to one or more of SEQ ID NOs: 101-104. In some embodiments, administration of the cytoplast expressing antigen derived from the hantavirus to a subject is therapeutically effective to confer immunity against an infection by the hantavirus, or reduce disease severity caused by the hantavirus, in the subject.

In some embodiments, the viral antigen is derived from a human immunodeficiency virus (HIV). In some embodiments, In some embodiments, the HIV antigen comprises an amino acid sequence that is at least or equal to about 50%, 60%, 70%, 75%, 80%, 85%, 90%, 95%, or 99% identical to one or more of SEQ ID NOs: 651-660. In some embodiments, the HIV antigen is encoded from a nucleic acid sequence that is at least or equal to about 50%, 60%, 70%, 75%, 80%, 85%, 90%, 95%, or 99% identical to one or more of SEQ ID NOs: 601-610. In some embodiments, administration of the cytoplast expressing antigen derived from the HIV to a subject is therapeutically effective to confer immunity against an infection by the HIV, or reduce disease severity caused by the HIV, in the subject.

In some embodiments, the viral antigen is derived from a respiratory syncytial virus (RSV) such as RSV Memphis 37. In some embodiments, In some embodiments, the RSV antigen comprises an amino acid sequence that is at least or equal to about 50%, 60%, 70%, 75%, 80%, 85%, 90%, 95%, or 99% identical to one or more of SEQ ID NOs: 751-761. In some embodiments, the RSV antigen is encoded from a nucleic acid sequence that is at least or equal to about 50%, 60%, 70%, 75%, 80%, 85%, 90%, 95%, or 99% identical to one or more of SEQ ID NOs: 701-711. In some embodiments, administration of the cytoplast expressing antigen derived from the RSV to a subject is therapeutically effective to confer immunity against an infection by the RSV, or reduce disease severity caused by the RSV, in the subject.

In some embodiments, the cytoplast can comprise a plurality of viral antigens, where the viral antigens are same (e. g. the cytoplast comprising only Spike protein as the viral antigen). In some embodiments, the cytoplast can comprise a plurality of viral antigens, where the viral antigens are different. For example, a cytoplast can comprise viral antigens comprising a combination of Spike protein, Nucleocapsid protein, Membrane protein, or Envelop protein. In some embodiments, the cytoplast can comprise a combination of viral antigens that can be expressed on the surface of the cytoplast, encapsulated by the cytoplast, and/or secreted by the cytoplast.

In some embodiments, the antigen is derived from a bacterium. The bacterium may be a Gram-positive bacterium. In some embodiments, the bacterium is a Gram-negative bacterium. In some embodiments, the bacterium is a strain that is resistant to β-lactamase In some embodiments, the antigen is derived from Enterotoxigenic Escherichia coli (ETEC), Shiga toxin-producing Escherichia coli (STEC), Campylobacter jejuni, Pseudomonas aeruginosa, Acinetobacter baumannii, Streptococcus mutans, Helicobacter pylori, or Bacillus anthracis.

In some embodiments, the bacterial antigen is derived from Bacillus anthracis (e.g., Anthrax). In some embodiments, the bacterial antigen is more than or equal to about 50%, 60%, 70%, 75%, 80%, 85%, 90%, 95%, or 99% identical to protective antigen (PA), and two enzyme components, edema factor (EF) and lethal factor (LF). In some embodiments, the bacterial antigen comprises an amino acid sequence that is at least or equal to about 50%, 60%, 70%, 75%, 80%, 85%, 90%, 95%, or 99% identical to one or more of SEQ ID NOs: 1151-1153. In some embodiments, the bacterial antigen is encoded from a nucleic acid sequence that is at least or equal to about 50%, 60%, 70%, 75%, 80%, 85%, 90%, 95%, or 99% identical to one or more of SEQ ID NOs: 1101-1103. In some embodiments, administering the cytoplast expressing the bacterial antigen derived from Bacillus anthracis to a subject is therapeutically effective to immunize the subject from an infection by the Bacillus anthracis, or reduce severity of a disease or condition caused by an infection by the Bacillus anthracis.

In some embodiments, the bacterial antigen is derived from Clostridium. In some embodiments, In some embodiments, the clostridium antigen comprises an amino acid sequence that is at least or equal to about 50%, 60%, 70%, 75%, 80%, 85%, 90%, 95%, or 99% identical to one or more of SEQ ID NOs: 951-984. In some embodiments, the Clostridium antigen is encoded from a nucleic acid sequence that is at least or equal to about 50%, 60%, 70%, 75%, 80%, 85%, 90%, 95%, or 99% identical to one or more of SEQ ID NOs: 901-934. In some embodiments, administration of the cytoplast expressing antigen derived from the Clostridium to a subject is therapeutically effective to confer immunity against an infection by the Clostridium, or reduce disease severity caused by the Clostridium, in the subject.

In some embodiments, the vaccine antigen is derived from Ricin. In some embodiments, In some embodiments, the Ricin antigen comprises an amino acid sequence that is at least or equal to about 50%, 60%, 70%, 75%, 80%, 85%, 90%, 95%, or 99% identical to one or more of SEQ ID NOs: 1051-1057. In some embodiments, the Ricin antigen is encoded from a nucleic acid sequence that is at least or equal to about 50%, 60%, 70%, 75%, 80%, 85%, 90%, 95%, or 99% identical to one or more of SEQ ID NOs: 1001-1007. In some embodiments, administration of the cytoplast expressing antigen derived from the Ricin to a subject is therapeutically effective to confer immunity against or reduce toxic effect caused by the Ricin in the subject.

In some embodiments, the antigen can be a fusion protein, where any one of the protein described herein or a fragment thereof can be fused with another peptide. In some embodiments, the antigen described herein can be fused with a cell membrane protein or a transmembrane protein. Exemplary cell membrane protein or transmembrane protein can include CD63, CD81, CD82, CD47, heterotrimeric G proteins, MHC class I, integrins, transferrin receptor (TFR2), LAMP1/2, heparan sulfate proteoglycans, EMMPRIN, ADAM10, GPI-anchored 5′nucleotidase, CD73, complement-binding proteins CD55 and CD59, sonic hedgehog (SHH), TSPAN8, CD37, CD53, CD9, PECAM1, ERBB2, EPCAM, CD90, CD45, CD41, CD42a, Glycophorin A, CD14, MHC class II, CD3, Acetylcholinesterase/AChE-S, AChE-E, amyloid beta A4/APP, and multidrug resistance-associated protein.

In some embodiments, the antigen can be fused with glycosyl-phosphatidylinositol (GPI) or a B7-1 antigen (B7-1) cytoplasmic tail. In some embodiments, the antigen can be fused with albumin. In some embodiments, the antigen can be expressed along with a polypeptide comprising a molecular clamp. In some embodiments, the molecular clamp, when expressed along with antigen in the same cytoplast, keeps the antigen in a pre-fusion form. In some embodiments, the molecular clamp comprises the polypeptide encoding a pattern that repeats after every two, three, four, five, six, seven, eight, nine, 10, 11, 12, 13, 14, 15, or more amino acid residues. In some embodiments, the polypeptide encoding the molecular clam is at least seven, eight, nine, 10, 11, 12, 13, 14, 15, 16, 17, 18, 19, 20 or more amino acid residues in length. In some embodiments, the molecular clamp self-assembles into a twin helix with one strand going forward and the other in reverse. In some embodiments, the pairing of the amino acids in the strands is ensured by a pattern of hydrophobic and hydrophilic amino acids. In some embodiments, the pattern is arranged so that none of the clamp binds to the viral antigen. In some embodiments, the molecular clamp self-assembles into a stiff rod. In some embodiments, the molecular clamp is linked to the desired part of the viral antigen by a linker, which can serve other functions such as allowing the cytoplast expressing the molecular clamp to be purified from a mixture.

In some embodiments, the antigen is a tumor antigen, or portion thereof, such as alphafetoprotein (AFP), carcinoembryonic antigen (CEA), CA0125, MUC-1, epithelium tumor antigen (ETA). In some embodiments, the antigen comprises an amino acid sequence that is at least or equal to about 50%, 60%, 70%, 75%, 80%, 85%, 90%, 95%, or 99% identical to any cancer epitope that is commonly known. In some embodiments, administration of the cytoplast expressing the tumor antigen, or portion thereof, to a subject is therapeutically effective to immunize the subject against an infection by an oncovirus, or reduce the severity of the cancer caused by the oncovirus.

b. Heterologous Nucleic Acid

Described herein, in some embodiments, is a vaccine comprising at least one heterologous polynucleotide. Non-limiting examples of polynucleotides that may be heterologous include coding or non-coding regions of a gene or gene fragment, loci (locus) defined from linkage analysis, exons, introns, messenger RNA (mRNA), self-amplifying RNA, uridine containing RNA (uRNA), self-amplifying mRNA, transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components. In some embodiments, the antigen translated from the heterologous polynucleotide can induce immune response in the subject. In some embodiments, the antigen translated from the heterologous polynucleotide can confer adaptive immunity to infection caused by any one of the pathogen described herein in the subject. In some embodiments, the antigen translated from the heterologous polynucleotide can treat or prevent a pathogenic infection caused by any one of the pathogens described herein in the subject.

In some embodiments, the heterologous polynucleotide can encode one or more of the immune-modulators described herein. In some embodiments, the immune-modulators augment the immune response induced by any one of the antigens described herein. In some embodiments, the immune-modulator is Ii-key/MHC class II epitope peptide. In some embodiments, the immune-modulator is any one of the cytokines described herein. In some embodiments, the heterologous polynucleotide can encode one or more of the homing proteins or one or more of the homing receptors described herein. In some embodiments, the homing protein can be secreted by the cytoplast. In some embodiments, the homing receptor can be expressed on the surface of the cytoplast. In some embodiments, the one or more homing receptors can be specific to one or more ligands expressed on one or more cells in lymph tissue, cells in the lymph tissue can comprise endothelial cells, lymphocytes, macrophages, or reticular cells, or a combination thereof

In some embodiments, the heterologous polynucleotide can encode one or more of the targeting moieties described herein. In some embodiments, the heterologous polynucleotide can encode one or more of the immune-modulators described herein. In some embodiments, the heterologous polynucleotide can encode one or more of homing receptors described herein. In some embodiments, the heterologous polynucleotide can encode one or more of the homing proteins described herein. In some embodiments, the heterologous polynucleotide can encode one or more of the anti-viral compositions described herein.

In some embodiments, the heterologous polynucleotide comprises a heterologous DNA sequence encoding the viral antigen. In some embodiments, the heterologous DNA sequence encodes any one of orf1a, orf1ab, Spike protein (S protein), 3a, 3b, Envelope protein (E protein), Membrane protein (M protein), p6, 7a, 7b, 8b, 9b, Nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase). In some embodiments, the cytoplast comprises the heterologous DNA sequence encoding Spike protein or a fragment thereof. In some embodiments, the cytoplast comprises the heterologous DNA sequence encoding Nucleocapsid protein or a fragment thereof. In some embodiments, the cytoplast comprises the heterologous DNA sequence encoding Membrane protein or a fragment thereof. In some embodiments, the cytoplast comprises the heterologous DNA sequence encoding Envelope protein or a fragment thereof. In some embodiments, the heterologous polynucleotide can comprise one or more heterologous DNA sequences encoding one or more antigens. For example, the heterologous polynucleotide can encode an S protein antigen and a N protein antigen. In some embodiments, the heterologous DNA sequences can encode any one of the different viral antigens described herein. In some embodiments, the cytoplast transcribes and translates the heterologous DNA sequence into the viral antigen. In some embodiments, the cytoplast delivers the heterologous DNA sequence to target tissue, where the heterologous DNA sequence is transcribed and then translated into the viral antigen by the target tissue. In some embodiments, the heterologous polynucleotide comprises a plasmid comprising the heterologous DNA sequence encoding any one of the antigens described herein. In some embodiments, the cytoplast comprises a SARS-CoV-2 vaccine comprising a DNA vaccine (GX-19) comprising a nucleic acid encoding an antigen derived from Spike protein of SARS-CoV-2.

In some embodiments, the at least one heterologous polynucleotide is at least or equal to about 50%, 60%, 70%, 80%, 90%, 95%, or 99% identical to a fragment of any one of SEQ ID NOs: 4-7. In some embodiments, the at least one heterologous polynucleotide is about 100% identical to a fragment of any one of SEQ ID NOs: 4-7. In some embodiments, the at least one heterologous polynucleotide encodes a viral antigen that is at least or equal to about 50%, 60%, 70%, 80%, 90%, 95%, or 99% identical to a fragment of SEQ ID NO: 8. In some embodiments, the at least one heterologous polynucleotide encodes a viral antigen that is 100% identical to a fragment of SEQ ID NO: 8. In some embodiments, the at least one heterologous polynucleotide encodes a viral antigen that is at least or equal to about 50%, 60%, 70%, 80%, 90%, 95%, or 99% identical to a fragment of SEQ ID NO: 9. In some embodiments, the at least one heterologous polynucleotide encodes a viral antigen that is about 100% identical to a fragment of SEQ ID NO: 9. In some embodiments, the at least one heterologous polynucleotide encodes a viral antigen that is at least or equal to about 50%, 60%, 70%, 80%, 90%, 95%, or 99% identical to a fragment of SEQ ID NO: 10. In some embodiments, the at least one heterologous polynucleotide encodes a viral antigen that is about 100% identical to a fragment of SEQ ID NO: 10. In some embodiments, the at least one heterologous polynucleotide encodes a viral antigen that is at least or equal to about 50%, 60%, 70%, 80%, 90%, 95%, or 99% identical to a fragment of SEQ ID NO: 11. In some embodiments, the at least one heterologous polynucleotide encodes a viral antigen that is about 100% identical to a fragment of SEQ ID NO: 11.

In some embodiments, the heterologous polynucleotide comprises a heterologous RNA sequence encoding the viral antigen. In some embodiments, the heterologous RNA sequence comprises an mRNA sequence encoding the viral antigen In some embodiments, the mRNA encodes any one of orf1a, orf1ab, Spike protein (S protein), 3a, 3b, Envelope protein (E protein), Membrane protein (M protein), p6, 7a, 7b, 8b, 9b, Nucleocapsid protein (N protein), orf14, nsp1 (leader protein), nsp2, nsp3, nsp4, nsp5 (3C-like proteinase), nsp6, nsp7, nsp8, nsp9, nsp10 (growth-factor-like protein), nsp12 (RNA-dependent RNA polymerase, or RdRp), nsp13 (RNA 5′-triphosphatase), nsp14 (3′-to-5′ exonuclease), nsp15 (endoRNAse), and nsp16 (2′-O-ribose methyltransferase). In some embodiments, the cytoplast comprises mRNA encoding Spike protein or a fragment thereof. In some embodiments, the cytoplast comprises mRNA encoding Nucleocapsid protein or a fragment thereof. In some embodiments, the cytoplast comprises mRNA encoding Membrane protein or a fragment thereof. In some embodiments, the cytoplast comprises mRNA encoding Envelope protein or a fragment thereof. In some embodiments, the heterologous polynucleotide can comprise one or more mRNA sequences. In some embodiments, the mRNA sequences can encode any one of the different viral antigens described herein. In some embodiments, the cytoplast translates the mRNA into the viral antigen. In some embodiments, the cytoplast delivers the mRNA to target tissue, where the mRNA is translated into the viral antigen by the target tissue. In some embodiments, the mRNA is a self-amplifying mRNA (saRNA). In some embodiments, the mRNA comprises uridine (uRNA). In some embodiments, the cytoplast comprises a SARS-CoV-2 vaccine comprising an mRNA encoding the full-length, prefusion stabilized Spike (S) protein (mRNA-1273). In some embodiments, the heterologous polynucleotide comprises one or more heterologous RNA sequences encoding one or more of the antigens described herein. In some embodiments, the cytoplast comprises a SARS-CoV-2 vaccine (mRNA-LNP vaccine) comprising an mRNA encoding an antigen derived from a protein of SARS-CoV-2. The mRNA is encapsulated and delivered via the use of lipid nanoparticle.

In some embodiments, the cytoplast comprises DNA or RNA vectors comprising the at least one heterologous polynucleotide encoding the viral antigens. In some embodiments, the DNA or RNA vectors can be plasmids. In some embodiments, the DNA or RNA vectors can be viral vector. Viral vectors, and especially retroviral vectors, can be engineered to comprise nucleic acid sequence encoding any one of the viral antigen described herein and be delivered to the target tissue by the cytoplast. In some embodiments, the viral vectors can be derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), replication-deficient chimpanzee adenovirus, ChAdOx1, Newcastle disease virus vector, M2-deficient single replication (M2SR) influenza vector, pox vectors, parvoviral vectors, baculovirus vectors, measles viral vectors, vesicular stomatitis virus (VSV) vector, or herpes simplex virus vectors (HSVs). In some embodiments, the retroviral vectors include gamma-retroviral vectors such as vectors derived from the Moloney Murine Leukemia Virus (MoMLV, MMLV, MuLV, or MLV) or the Murine Steam cell Virus (MSCV) genome. In some embodiments, the retroviral vectors also include lentiviral vectors such as those derived from the human immunodeficiency virus (HIV) genome. In some embodiments, AAV vectors include AAV1, AAV2, AAV4, AAV5, AAV6, AAV7, AAV8, or AAV9 serotype. In some embodiments, the viral vector is a chimeric viral vector, comprising viral portions from two or more viruses. In additional instances, the viral vector is a recombinant viral vector. In some embodiments, the cytoplast comprises a SARS-CoV-2 vaccine (Gam-COVID-Vac or Gam-COVID-Vac lyo) non-replicating viral vector comprising nucleic acid encoding S protein or a fragment thereof of the SARS-CoV-2. In some embodiments, the cytoplast comprises a SARS-CoV-2 vaccine comprising an adenoviral vector comprising nucleic acid sequence the Spike (S) protein of SARS-CoV-2 (Ad5-nCoV). In some embodiments, the cytoplast comprises a SARS-CoV-2 vaccine comprising a replication-deficient chimpanzee adenovirus, ChAdOxl, which is engineered to express the Spike (S) protein of SARS-CoV-2. In some embodiments, the cytoplast comprises a SARS-CoV-2 vaccine comprising a non-replicative adenoviral vector (AdVac) comprising nucleic acid encoding an antigen derived from a protein of SARS-CoV-2. In some embodiments, the AdVac vaccine is prepared with PER.C6 cells. In some embodiments, the cytoplast comprises a SARS-CoV-2 vaccine comprising INO-4800 pGX DNA plasmid with nucleic acid encoding the Spike (S) protein of SARS-CoV-2 as the insert. In some embodiments, the cytoplast comprises a SARS-CoV-2 vaccine comprising mRNA or modified mRNA to express the Spike (S) protein or a fragment thereof of SARS-CoV-2 (BNT162). In some embodiments, the cytoplast comprises a SARS-CoV-2 vaccine comprising a measles vector comprising nucleic acid encoding the Spike protein or a fragment thereof of SARS-CoV-2. In some embodiments, the cytoplast comprises a SARS-CoV-2 vaccine comprising DNA encoding the Spike protein delivered to the muscle of the subject via injection followed by electroporation.

c. Inactivated Pathogen and Portions Thereof

In some embodiments, the cytoplast comprises an inactivated pathogen (e.g., virus, bacterium, parasite, or fungus), or portion thereof. In some embodiments, the inactivated pathogen is an inactivated virus or a portion thereof. In some embodiments, the inactivated virus is any one of the viruses described herein. In some embodiments, the inactivated virus is derived from a coronavirus, a hantavirus, an ebolavirus, an influenza virus, a respiratory syncytial virus, a rotavirus, a norovirus, a hepatitis virus, or porcine reproductive and respiratory syndrome virus. In some embodiments, the inactivated virus is derived from a coronavirus. In some embodiments, the inactivated virus is a betacoronavirus such as a SARS-CoV-2. In some embodiments, the inactivated virus is inactivated SARS-CoV-2.

In some embodiments, the cytoplast comprises inactivated SARS-CoV-2. In some embodiments, the SARS-CoV-2 comprises a mutation comprising Asp614Gly, Pro323Leu, Ile599Val, pro585Ser, Phe308Tyr, Thr141Ile, Asp248Glu, Thr85Ile, Ala18Val, Asn439Lys, Glu251Val, Pro10ser, Ser194Leu, Ser197Leu, Gly196Val, Leu108Phe, Gln213Lys, Leu84Ser, Thr175Met, Ser563Leu, Val13Leu, Gln57His, or Thr14Ile, as compared with the full-length amino acid sequence for the Wuhan strain.

In some embodiments, the cytoplast comprising inactivated SARS-CoV-2 induces immune response and adaptive immunity towards SARS-CoV-2 in a subject when the cytoplast comprising the inactivated SARS-CoV-2 is engulfed by immune cell of the subject. Upon engulfing the cytoplast, the immune cell contacts the inactivated SARS-CoV-2 and subsequently develops adaptive immune response towards SARS-CoV-2. In some embodiments, the inactivated SARS-CoV-2 virus is formalin-inactivated SARS-CoV-2 virus. In some embodiments, the cytoplast comprises a SARS-CoV-2 vaccine (PiCoVacc) comprising a formalin-inactivated SARS-CoV-2 virus, obtained from vero cell culture. In some embodiments, the cytoplast comprises a SARS-CoV-2 vaccine comprising Bacille Calmette-Guérin (BCG). In some embodiments, the cytoplast comprises a SARS-CoV-2 vaccine (bacTRL-Spike) comprising bifidobacterial engineered to express the Spike protein of SARSO-CoV-2. In some embodiments, the cytoplast comprises a SARS-CoV-2 vaccine (PittCoVacc) comprising delivering the Spike (S) protein or a fragment thereof of SARS-CoV-2 via the use of microneedle array. In some embodiments, the cytoplast comprises a SARS-CoV-2 vaccine (NVX-CoV2373) comprising a multiple recombinant nanoparticle vaccine comprising a prefusion form of the Spike protein of SARS-CoV-2. In some embodiments, the cytoplast comprising the NVX-CoV2373 comprises an adjuvant or an immune-modulator. In some embodiments, the cytoplast comprises a SARS-CoV-2 vaccine comprising virus-like particle (VLP) mimicking the viral structure of SARS-CoV-2, where the VLP is manufactured from plant-based production methods. In some embodiments, the cytoplast comprises a SARS-CoV-2 vaccine (LUNAR-COV19) comprising an mRNA encoding the Spike protein of SARS-CoV-2. The mRNA is encapsulated and delivered via the use of lipid-mediated delivery system. In some embodiments, the cytoplast comprises a SARS-CoV-2 vaccine comprising an antigen derived from a Spike protein, said vaccine further comprising gp96 and OX40L, co-stimulators of T cell. In some embodiments, the cytoplast comprises a SARS-CoV-2 vaccine (T-COVIDTM)) comprising replication-deficient adenovirus 5 (RD-Ad5) vector comprising nucleic acid encoding Spike protein or a fragment thereof of SARS-CoV-2, where the T-COVIDTM vaccine is formulated for intranasal delivery. In some embodiments the cytoplast comprising a SARS-CoV-2 vaccine is formulated for administration via any suitable route, e.g., subcutaneous, intravenous, arterial, ocular, oral, intramuscular, intranasal (e.g., inhalation), intraperitoneal, topical, mucosal, epidural, sublingual, epicutaneous, extra-amniotic, inter-articular, intradermal, intraosseous, intrathecal, intrauterine, intravaginal, intravesical, intravitreal, perivascular, and/or rectal administration, or any combination of known administration methods.

In some embodiment the inactivated virus is derived from a virus that causes viral hemorrhagic fevers, including Filoviruses (Ebola, Marburg) and Arenaviruses (Lassa, Machupo). In some embodiments, the inactivated virus is derived from a virus that causes viral encephalitis (alphaviruses, such as eastern equine encephalitis, Venezuelan equine encephalitis, and western equine encephalitis). In some embodiments, the inactivated virus is derived from a hantavirus, an ebolavirus, an influenza virus, a respiratory syncytial virus, a rotavirus, a norovirus, a hepatitis virus, or porcine reproductive and respiratory syndrome virus.

In some embodiments, the inactivated pathogen is an inactivated bacterium, or portion thereof. In some embodiments, the antigen is derived from an inactivated bacterium. The inactivated bacterium may be derived from a Gram-positive bacterium. In some embodiments, the inactivated bacterium is derived from a Gram-negative bacterium. In some embodiments, inactivated bacterium is derived from a strain that is resistant to B-lactamase In some embodiments, the inactivated bacterium is derived from Enterotoxigenic Escherichia coli (ETEC), Shiga toxin-producing Escherichia coli (STEC), Campylobacter jejuni, Pseudomonas aeruginosa, Acinetobacter baumannii, Streptococcus mutans, Helicobacter pylori, or Bacillus anthracis. In some embodiments, the inactivated bacterium is derived from a bacterium of Brucellosis (Brucella species), Epsilon toxin of Clostridium perfringens. Food safety threats (Salmonella species, Escherichia coli O 157:H7, Shigella), Glanders (Burkholderia mallei), Melioidosis (Burkholderia pseudomallei), Psittacosis (Chlamydia psittaci), Q fever (Coxiella burnetii), Ricin toxin from Ricinus communis (castor beans), Staphylococcal enterotoxin B, Typhus fever (Rickettsia prowazekii), Water safety threats (Vibrio cholerae, Cryptosporidium parvum), Anthrax (Bacillus anthracis), Botulism (Clostridium botulinum toxin), Plague (Yersinia pestis), Smallpox (variola major), or Tularemia (Francisella tularensis) 2. Additional Exogenous Agents

Cytoplasts of the present disclosure may be engineered to express an additional exogenous agent such as an immune modulator. In some embodiments, the cytoplast comprises one or more immune-modulators described herein. An immune-modulator may be a molecule that directly or indirectly stimulates an immune response in a subject. In some embodiments, the immune-modulator may be an immune activator to elicit an adaptive immune response in the subject. In some embodiments, the immune activator may be an immune suppressor to suppress an overactive immune system in a subject, for example, a subject with a proliferative disease or disorder. In some embodiments, the immune-modulator may be expressed on the surface of the cytoplast. In some embodiments, the immune-modulator may be released by the cytoplast. In some embodiments, the immune-modulator may be secreted by the cytoplast. In some embodiments, the immune-modulator may be a cargo of the cytoplast. In some embodiments, the immune-modulator maybe a peptide or protein that is fused with the antigen described herein. In some embodiments, the immune-modulator may be an adjuvant.

In a non-limiting example, the immune-modulator may directly stimulates an immune response by binding to a cognate receptor on the surface of immune cells, which causes the immune cells to release cytokines, thereby activating the immune cells. Activation of immune cells, in some embodiments, facilitates the development of adaptive immunity against the virus. As another example, an immune-modulator indirectly stimulates an immune response by suppressing IL-10 production and secretion by the target cell and/or by suppressing the activity of regulatory T cells, resulting in, for example, an increased anti-tumor response by immune cells. By contrast, an immune-modulator acting as an immune suppressor can directly or indirectly inhibit an immune response in the subject.

In certain embodiments, an immune-modulator targets a pattern recognition receptor (PRR). These receptors can be transmembrane or intra-endosomal proteins which can prime activation of the immune system in response to infectious agents such as pathogens. PRRs can recognize pathogen-associated molecular patterns (PAMPs) molecules and damage-associated molecular patterns (DAMPs) molecules. A PRR can be membrane bound. A PRR can be cytosolic. Membrane-bound PRRs include toll-like receptors and C-type lectin receptors, such as mannose receptors and asialoglycoprotein receptors. Cytoplastic PRRs include NOD-like receptors, and RIG-I-like receptors.

In certain embodiments, an immune-modulator is a Damage-Associated Molecular Pattern (DAMP) molecule or a Pathogen-Associated Molecular Pattern (PAMP) molecule, such as a DAMP agonist or a PAMP agonist. DAMP molecules and PAMP molecules can be recognized by receptors of the innate immune system, such as Toll-like receptors (TLRs), Nod-like receptors, C-type lectins, and RIG-I-like receptors. In certain embodiments, an immune-modulatory agent is a Toll-like receptor agonist, a STING agonist, or a RIG-I agonist. Examples of DAMP molecules can include proteins such as chromatin-associated protein high-mobility group box 1 (HMGB1), S100 molecules of the calcium modulated family of proteins and glycans, such as hyaluronan fragments, and glycan conjugates. DAMP molecules can also be nucleic acids, such as DNA, when released from tumor cells following apoptosis or necrosis. Examples of additional DAMP nucleic acids can include RNA and purine metabolites, such as ATP, adenosine and uric acid, present outside of the nucleus or mitochondria.

In some embodiments, an immune-modulator is a cytosolic DNA and bacterial nucleic acids called cyclic dinucleotides, that are recognized by Interferon Regulatory Factor (IRF) or stimulator of interferon genes (STING), which can act a cytosolic DNA sensor. Compounds recognized by Interferon Regulatory Factor (IRF) can play a role in immunoregulation by TLRs and other pattern recognition receptors.

An immune-modulator can be a toll-like receptor (TLR) agonist. An immune-modulatory agent can be RIG-I-like receptor ligand. An immune-modulatory agent can be a C-type lectin receptor ligand. An immune-modulatory agent can be a NOD-like receptor ligand.

In some embodiments, an immune-modulator is a TLR agonist. In some embodiments, an immune-modulator is selected the group consisting of a TLR1, TLR2, TLR3, TLR4, TLR5, TLR6, TLR7, TLR8, TLR9, TLR10, TLR11, TLR12 or TLR13 agonist, according the animal species.

In some embodiments, an immune-modulator activator is a ligand of TLR2 comprising: (a) a heat killed bacteria product, preferably HKAL, HKEB, HKHP, HKLM, HKLP, HKLR, HKMF, HKPA, HKPG, or HKSA, HKSP, and (b) a cell-wall components product, preferably LAM, LM, LPS, LIA, LIA, PGN, FSL, Pam2CSK4, Pam3CSK4, or Zymosan.

In some embodiments, an immune-modulator is a ligand of TLR3 selected from the group consisting of: rintatolimod, poly-ICLC, RIBOXXON®, Apoxxim, RIBOXXIM®, IPH-33, MCT-465, MCT-475, and ND-1.1.

In some embodiments, an immune-modulator is a ligand of TLR4 selected from the group consisting of LPS, MPLA or a pyrimido[5,4-b]indole such as those described in WO 2014/052828 (U of Cal), AZ126 (N-(2-(cyclopentylamino)-2-oxo-1-(pyridin-4-yl)ethyl)-N-(4-methoxyphenyl)-3-methyl-5-phenyl-1H-pyrrole-2-carboxamide) or AZ368 ((E)-3-(4-(2-(cyclopentylamino)-1-(N-(4-isopropylphenyl)-1,5-diphenyl-1H-pyrazole-3-carboxamido)-2-oxoethyl)phenyl)acrylic acid).

In some embodiments, an immune-modulator is a ligand of TLR5 selected from the group consisting of: FLA and Flagellin. In some embodiments, an immune-modulator is a ligand of TLR6. In certain embodiments, an immune-modulator is a TLR7 agonist and/or a TLR8 agonist. In certain embodiments, an immune-modulator is a TLR7 agonist. In certain embodiments, an immune-modulator is a TLR8 agonist. In some embodiments, an immune-modulator selectively agonizes TLR7 and not TLR8. In other embodiments, an immune-stimulator agonizes TLR8 and not TLR7.

In certain embodiments, an immune-modulator is a TLR7 agonist. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In certain embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine, but is other than a guanosine analog, an adenosine analog, a thymidine homopolymer, ssRNA, CpG-A, PolyG10, and PolyG3. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences). In some embodiments, a TLR7 agonist has an EC50 value of 500 nM or less by PBMC assay measuring TNFalpha or IFNalpha production. In some embodiments, a TLR7 agonist has an EC50 value of 100 nM or less by PBMC assay measuring TNFalpha or IFNalpha production. In some embodiments, a TLR7 agonist has an EC50 value of 50 nM or less by PBMC assay measuring TNFalpha or IFNalpha production. In some embodiments, a TLR7 agonist has an EC50 value of 10 nM or less by PBMC assay measuring TNFalpha or IFNalpha production.

In certain embodiments, an immune-modulator is a TLR8 agonist. In certain embodiments, the TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In certain embodiments, a TLR8 agonist is selected from the group consisting of a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine and is other a ssRNA. In some embodiments, an immune-modulator is a TLR8 agonist, other than a naturally occurring TLR8 agonist or a benzazepine agonist of TLR8.

In one embodiment, the cytoplast described herein can express and/or secret at least one immune-modulator comprising a co-stimulatory ligand which is a non-antigen specific signal important for full activation of an immune cell. Co-stimulatory ligands include, without limitation, tumor necrosis factor (TNF) ligands, cytokines (such as IL-2, IL-12, 1L-15 or IL21), and immunoglobulin (Ig) superfamily ligands. Tumor necrosis factor (TNF) is a cytokine involved in systemic inflammation and stimulates the acute phase reaction. Its primary role is in the regulation of immune cells. Tumor necrosis factor (TNF) ligands share a number of common features. The majority of the ligands are synthesized as type II transmembrane proteins containing a short cytoplasmic segment and a relatively long extracellular region. TNF ligands include, without limitation, nerve growth factor (NGF), CD4OL (CD4OL)/CD154, CD137L/4-1BBL, tumor necrosis factor alpha (TNFa), CD134L/OX4OL/CD252, CD27L/CD70, Fas ligand (FasL), CD3OL/CD153, tumor necrosis factor f3 (TNF(3)/lymphotoxin-alpha (LTa), lymphotoxin-beta (ur(3), CD257/B cell-activating factor (BAFF)/Blys/THANK/Ta11-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins, they possess an immunoglobulin domain (fold). Immunoglobulin superfamily ligands include, without limitation, CD80 and CD86, both ligands for CD28.

In some embodiments, the immune-modulator can be an adjuvant. In some embodiments, the adjuvant can comprise analgesic adjuvants. In some embodiments, the adjuvant can comprise inorganic compounds such as alum, aluminum hydroxide, aluminum phosphate, or calcium phosphate hydroxide. In some embodiments, the adjuvant can comprise mineral oil or paraffin oil. In some embodiments, the adjuvant can comprise bacterial products such as inactivated Bordetella pertussis, Mycobacterium bovis, tor oxoids. In some embodiments, the adjuvant can comprise nonbacterial organics like squalene. In some embodiments, the adjuvant can comprise the use of delivery systems such as detergents (Quil A). In some embodiments, the adjuvant can comprise plant saponins such as saponin derived from Quillaja, soybean, or Polygala senega. In some embodiments, the adjuvant can comprise Freund's complete adjuvant or Freund's incomplete adjuvant. In some embodiments, the adjuvant can comprise food-based oil like peanut oil.

In some embodiments, the cytoplast comprises one or more additional therapeutic agents such as an anti-viral composition described herein. In some embodiments, the one or more additional therapeutic agents may be any one of or any combination of a therapeutic DNA molecule, a therapeutic RNA molecule, a therapeutic protein (e.g., an enzyme, an antibody, an antigen, a toxin, cytokine, a protein hormone, a growth factor, a cell surface receptor, or a vaccine), a therapeutic peptide (e.g., a peptide hormone or an antigen), a small molecule active agent (e.g., a steroid, a polyketide, an alkaloid, a toxin, an antibiotic, an antiviral, a colchicine, a taxol, a mitomycin, or emtansine), and a therapeutic gene editing factor.

D. Pharmaceutic Compositions, Formulations, Dosages, and Routes of Administration

Provided herein are pharmaceutical compositions that include a cytoplast (e.g., a cytoplast obtained from any cell described herein). In some embodiments, the compositions are formulated for different routes of administration (e.g., intravenous, subcutaneous, intramuscular, retro-orbital, intraperitoneal, intra-lymph node). In some embodiments, the compositions can include a pharmaceutically acceptable carrier (e.g., phosphate buffered saline). The term “pharmaceutical composition” refers to a mixture of a cytoplast disclosed herein with other chemical components, such as diluents or carriers. The pharmaceutical composition can facilitate administration of the compound to an organism.

In general, methods disclosed herein comprise administering a cytoplast composition by systemic administration. In some embodiments, methods comprise administering a cytoplast composition by oral administration. In some embodiments, methods comprise administering a cytoplast composition by intraperitoneal injection. In some embodiments, methods comprise administering a cytoplast composition in the form of an anal suppository. In some embodiments, methods comprise administering a cytoplast composition by intravenous (“i.v.”) administration. It is conceivable that one may also administer cytoplast compositions disclosed herein by other routes, such as subcutaneous injection, intramuscular injection, intradermal injection, transdermal injection percutaneous administration, intranasal administration, intralymphatic injection, rectal administration intragastric administration, intraocular administration, intracerebro-ventricular administration, intrathecally, or any other suitable parenteral administration. In some embodiments, routes for local delivery closer to site of injury or inflammation are preferred over systemic routes. Routes, dosage, time points, and duration of administrating therapeutics may be adjusted. In some embodiments, administration of therapeutics is prior to, or after, onset of either, or both, acute and chronic symptoms of the pathogen-associated disease or condition.

An effective dose and dosage of the cytoplasts disclosed herein to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the cytoplast composition is not therapeutically effective or is not providing a sufficient alleviation of the disease or condition, or symptom of the disease or condition, then the dosage amount and/or route of administration may be changed, or an additional agent may be administered to the subject, along with the cytoplast composition. In some embodiments, as a patient is started on a regimen of a cytoplast composition, the patient is also weaned off (e.g., step-wise decrease in dose) a second treatment regimen.

Disclosed herein, in some embodiments are formulations of pharmaceutically-acceptable excipients and carrier solutions suitable for delivery of the cytoplast composition described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In some embodiments, the cytoplast composition are suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection.

In some embodiments, the pharmaceutical forms of the cytoplast compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In some embodiments, for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Hydrocarbon propellants useful include, for example, propane, isobutane, n-butane, pentane, isopentane and neopentane. A blend of hydrocarbons can also be used as a propellant. Ether propellants include, for example, dimethyl ether as well as the ethers. An aerosol formulation can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present disclosure can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent such as a transporter, carrier, or ion channel inhibitor in (substantially) pure propellant or as a mixture of propellant and solvent. The solvent can be used to dissolve the agent and/or retard the evaporation of the propellant. Solvents can include, for example, water, ethanol and glycols. Any combination of suitable solvents can be use, optionally combined with preservatives, antioxidants, and/or other aerosol components.

An aerosol formulation can be a dispersion or suspension. A suspension aerosol formulation can comprise a suspension of an agent or combination of agents, e.g., a transporter, carrier, or ion channel inhibitor, and a dispersing agent. Dispersing agents can include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents, e.g., a transporter, carrier, or ion channel. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

Disclosed herein are sterile injectable solutions comprising the cytoplast composition disclosed herein, which are prepared by incorporating the cytoplast composition disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In some embodiments, the compositions disclosed herein may also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Suitable dose and dosage administrated to a subject is determined by factors including, but not limited to, the particular cytoplast composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount cytoplast compositions and time of administration of such compositions will be within the purview of the skilled artisan having benefit of the present teachings. It is likely, however, possible that administration of therapeutically-effective amounts of the disclosed compositions may be achieved by a single administration, such as for example, a single injection of sufficient numbers of cytoplasts to provide therapeutic benefit to the patient undergoing such treatment.

Alternatively, in some circumstances, it may be desirable to provide multiple, or successive administrations of the cytoplast compositions, either over a relatively short, or a relatively prolonged period of time, as may be determined by the medical practitioner overseeing the administration of such compositions. For example, the number of cytoplasts administered to a mammal may be on the order of about 10⁷, 10⁸, 10⁹, 10¹⁰, 10¹¹, 10¹², 10¹³, or even higher, cytoplasts given either as a single dose, or divided into two or more administrations as may be required to achieve therapy of the particular disease or disorder being treated. In fact, in certain embodiments, it may be desirable to administer two or more different cytoplast compositions, either alone, or in combination with one or more other therapeutic drugs to achieve the desired effects of a particular therapy regimen. In various embodiments, the daily and unit dosages are altered depending on a number of variables including, but not limited to, the activity of the cytoplast composition used, the disease or condition to be treated, the mode of administration, the requirements of the individual subject, the severity of the disease or condition being treated, and the judgment of the practitioner.

In some embodiments, the administration of the cytoplast composition is hourly, once every 2 hours, 3 hours, 4 hours, 5 hours, 6 hours,? hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges may be adjusted based on subject's response to the treatment. Some routes of administration will require higher concentrations of effective amount of therapeutics than other routes.

Although not anticipated given the advantages of the present disclosure, in certain embodiments wherein the patient's condition does not improve, upon the doctor's discretion the administration of cytoplast composition is administered chronically, that is, for an extended period of time, including throughout the duration of the patient's life in order to ameliorate or otherwise control or limit the symptoms of the patient's disease or condition. In certain embodiments wherein a patient's status does improve, the dose of cytoplast composition being administered may be temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the dosage amount of the cytoplast composition described herein lies within a range of circulating concentrations that include the ED50 with minimal toxicity. In certain embodiments, the daily dosage range and/or the unit dosage amount varies within this range depending upon the dosage form employed and the route of administration utilized.

E. Pathogen Trapping Cytoplasts

Disclosed herein, in some embodiments, is a cytoplast engineered to trap a pathogen by permitting the pathogen to infect the cytoplast and preventing the pathogen from propagating or replicating within the cytoplast. The controllable and finite lifespan of the cytoplast enables the cytoplast to kill the pathogen when the cytoplast dies having the pathogen trapped in the cytoplast at death. Death of the cytoplast can be a natural process, such through apoptosis or autophagy. The cytoplasts engineered to trap a pathogen can be engineered to express pathogen-recognized moieties, such as a host receptor, that encourages infection of the cytoplast by the pathogen. In addition, or alternatively, the cytoplast can be engineered to express or contain an active agent described herein that is therapeutically effective to treat or prevent an infection by the pathogen in cell of a subject. Such active agents, for example, can be neutralizing antibodies that, when secreted from the cytoplast, functionally block binding between the pathogen in extracellular space and host cells. In the case of preventing an infection by a SARS-CoV-2, the neutralizing antibodies block binding between the SARS-CoV-2 spike protein and the human angiotensin-converting enzyme 2 (ACE2) expressed on the host cell to prevent infection.

A pathogen can be any bacteria, virus, or fungus that can infect a cell described herein that, at least partially, requires nuclear genetic information to replicate or propagate, such as those disclosed herein. The infected cytoplast lacks nuclear components needed for replication or propagation of pathogens that have replicative stages in the nuclei of a host cell, thus decreasing preventing or treating the infection by the pathogen in a subject.

In the case for reducing or preventing an infection by SARS-CoV-2, the cytoplast is engineered to express a pathogen-recognized moiety for SARS-CoV-2 (e.g., ACE2), and when the cytoplast is infected by SARS-CoV-2 via spike protein and ACE2 binding, the cytoplast can naturally, or be engineered to, recruit macrophages for macrophage phagocytosis. As seen in FIG. 4, as a non-limiting example, the phagocytosis of the infected cytoplast can activate immune cells such as helper T cells and B cells to generate antibodies against SARS-CoV-2. In some embodiments, the phagocytosis of the infected cytoplast can activate T cells for treating the viral infection.

The cytoplasts described herein, in some embodiments, are engineered to express, and in some cases, display a pathogen-recognized moiety. In some embodiments, the pathogen-recognized moiety is a host receptor (a cognate receptor for the pathogen of interest), or a portion thereof sufficient to facilitate binding between the pathogen and the host cell. The pathogen-recognized moiety may be expressed by the cytoplast on the surface of the cytoplast. In some embodiments, the pathogen-recognized moiety is derived from a protein that is at least partially exposed to an extracellular environment. In some embodiments, the pathogen-recognized moiety is derived from a polypeptide encoding a cell surface receptor or a transmembrane protein. In some embodiments, pathogen-recognized moiety is derived from a protein that is bound by a viral protein during viral infection. For example, the pathogen-recognized moiety may be derived from the Angiotensin I Converting Enzyme 2 (ACE2), which is bound by the Spike protein of the SARS-CoV-2 during viral infection. In some embodiments, the pathogen-recognized moiety is derived from a cell surface receptor or a transmembrane protein that can be recognized and bound by any one of the virus described herein. In some embodiments, the pathogen-recognized moiety is derived from a cell surface receptor or a transmembrane protein that can be recognized and bound by any one of the coronavirus described herein. In some embodiments, the pathogen-recognized moiety is a sugar. In some embodiments, the pathogen-recognized moiety is a polypeptide. Non-limiting receptors that are recognized by a coronavirus include ACE2, Alanine aminopeptidase (ANPEP), Carcinoembryonic antigen-related cell adhesion molecule (CEACAM1), Dipeptidyl peptidase-4 (DPP4), or a sugar.

In some embodiments, the cytoplast is engineered to express human angiotensin-converting enzyme 2 (ACE2), or a portion thereof, which can be recognized and bound by a coronavirus specific to ACE2, such as for example, SARS-CoV, SARS-CoV-2, and NL63. In some embodiments, the cytoplast is engineered to express the ACE2, or portion thereof, on the surface of the cytoplast. In some embodiments, the cytoplast is engineered to express full length of ACE2. In some embodiments, the cytoplast is engineered to express a fragment of ACE2. In some embodiments, the portion of the ACE2 comprises between about 5 amino acids to about 805 amino acids of an amino acid sequence of the ACE2 polypeptide. In some embodiments, the pathogen-recognized moiety comprising the portion of the ACE2 is derived from the extracellular domain or the portion of the ACE2 that is expressed on the outside of the cell. In some embodiments, the portion of the ACE2 comprises a N-terminus portion of the amino acid sequence of ACE2. In some embodiments, the portion of the ACE2 comprises a C-terminus portion of the amino acid sequence of ACE2. In some embodiments, the portion of the ACE2 comprises an amino acid sequence of the ACE2 polypeptide comprising between about 5 amino acids to about 10 amino acids, about 5 amino acids to about 15 amino acids, about 5 amino acids to about 20 amino acids, about 5 amino acids to about 25 amino acids, about 5 amino acids to about 50 amino acids, about 5 amino acids to about 100 amino acids, about 5 amino acids to about 200 amino acids, about 5 amino acids to about 400 amino acids, about 5 amino acids to about 500 amino acids, about 5 amino acids to about 600 amino acids, about 5 amino acids to about 805 amino acids, about 10 amino acids to about 15 amino acids, about 10 amino acids to about 20 amino acids, about 10 amino acids to about 25 amino acids, about 10 amino acids to about 50 amino acids, about 10 amino acids to about 100 amino acids, about 10 amino acids to about 200 amino acids, about 10 amino acids to about 400 amino acids, about 10 amino acids to about 500 amino acids, about 10 amino acids to about 600 amino acids, about 10 amino acids to about 805 amino acids, about 15 amino acids to about 20 amino acids, about 15 amino acids to about 25 amino acids, about 15 amino acids to about 50 amino acids, about 15 amino acids to about 100 amino acids, about 15 amino acids to about 200 amino acids, about 15 amino acids to about 400 amino acids, about 15 amino acids to about 500 amino acids, about 15 amino acids to about 600 amino acids, about 15 amino acids to about 805 amino acids, about 20 amino acids to about 25 amino acids, about 20 amino acids to about 50 amino acids, about 20 amino acids to about 100 amino acids, about 20 amino acids to about 200 amino acids, about 20 amino acids to about 400 amino acids, about 20 amino acids to about 500 amino acids, about 20 amino acids to about 600 amino acids, about 20 amino acids to about 805 amino acids, about 25 amino acids to about 50 amino acids, about 25 amino acids to about 100 amino acids, about 25 amino acids to about 200 amino acids, about 25 amino acids to about 400 amino acids, about 25 amino acids to about 500 amino acids, about 25 amino acids to about 600 amino acids, about 25 amino acids to about 805 amino acids, about 50 amino acids to about 100 amino acids, about 50 amino acids to about 200 amino acids, about 50 amino acids to about 400 amino acids, about 50 amino acids to about 500 amino acids, about 50 amino acids to about 600 amino acids, about 50 amino acids to about 805 amino acids, about 100 amino acids to about 200 amino acids, about 100 amino acids to about 400 amino acids, about 100 amino acids to about 500 amino acids, about 100 amino acids to about 600 amino acids, about 100 amino acids to about 805 amino acids, about 200 amino acids to about 400 amino acids, about 200 amino acids to about 500 amino acids, about 200 amino acids to about 600 amino acids, about 200 amino acids to about 805 amino acids, about 400 amino acids to about 500 amino acids, about 400 amino acids to about 600 amino acids, about 400 amino acids to about 805 amino acids, about 500 amino acids to about 600 amino acids, about 500 amino acids to about 805 amino acids, or about 600 amino acids to about 805 amino acids. In some embodiments, the portion of the ACE2 comprises between about 5 amino acids, about 10 amino acids, about 15 amino acids, about 20 amino acids, about 25 amino acids, about 50 amino acids, about 100 amino acids, about 200 amino acids, about 400 amino acids, about 500 amino acids, about 600 amino acids, or about 805 amino acids, of the amino acid sequence of the ACE2 polypeptide. In some embodiments, the portion of the ACE2 comprises at least or equal to about 5 amino acids, about 10 amino acids, about 15 amino acids, about 20 amino acids, about 25 amino acids, about 50 amino acids, about 100 amino acids, about 200 amino acids, about 400 amino acids, about 500 amino acids, or about 600 amino acids, of the amino acid sequence of the ACE2 polypeptide. In some embodiments, the portion of the ACE2 comprises at most about 10 amino acids, about 15 amino acids, about 20 amino acids, about 25 amino acids, about 50 amino acids, about 100 amino acids, about 200 amino acids, about 400 amino acids, about 500 amino acids, about 600 amino acids, or about 805 amino acids, of the amino acid sequence of the ACE2 polypeptide. In some embodiments, the ACE2 is human ACE2 (huACE2). In some embodiments, the amino acid sequence for huACE2 is provided in SEQ ID NO: 12.

In some embodiments, the cytoplast is engineered to express a heterologous polypeptide that is at least or equal to 50%, 60%, 70%, 80%, 90%, 95%, or 99% identical to SEQ ID NO: 12. In some embodiments, the cytoplast is engineered to express a heterologous polypeptide that is at least or equal to 50%, 60%, 70%, 80%, 90%, 95%, or 99% identical to a fragment of SEQ ID NO: 12. In some embodiments, the cytoplast is engineered to express a heterologous polypeptide that is 100% identical to SEQ ID NO: 12. In some embodiments, the cytoplast is engineered to express a heterologous polypeptide that is 100% identical to a fragment of SEQ ID NO: 12.

In some embodiments, the cytoplast can be engineered to express more ACE2 compared to a cell that expresses ACE2 at an endogenous level and can be infected by SARS-CoV-2. In some embodiments, the cytoplast can express at least or equal to 10%, 20%, 30% 40%, 50%, 60%, 70%, 80%, 90%, 95%, 99%, or more ACE2 compared to the cell expressing the ACE2 at the endogenous level. In some embodiments, the cytoplast can express at least or equal to 2 folds, 5 folds, 10 folds, 50 folds, 100 folds, 500 folds, 1000 folds, 5000 folds, 10000 folds, or more folds ACE2 compared to the cell that expresses ACE at the endogenous level and can be infected by SARS-CoV-2. In some embodiments, the cytoplast can be engineered to express more ACE2 on the surface of the cytoplast compared to a cell expressing ACE2 at the endogenous level on the surface of the cell. In some embodiments, the cytoplast can express at least or equal to 10%, 20%, 30% 40%, 50%, 60%, 70%, 80%, 90%, 95%, 99%, or more ACE2 on the surface or the cytoplast compared to the cell expressing ACE2 at the endogenous level on the surface of the cell. In some embodiments, the cytoplast can express at least or equal to 2 folds, 5 folds, 10 folds, 50 folds, 100 folds, 500 folds, 1000 folds, 5000 folds, 10000 folds, or more folds of ACE2 on the surface of the cytoplast compared to the cell expressing ACE2 at the endogenous level on the surface of the cell.

In some embodiments, the cytoplast expressing ACE2 can have higher viral infectivity as compared to a reference cell. A “reference cell” in this context can be a naturally occurring cell capable of being infected by SARS-CoV-2 (e.g., naturally expresses ACE2). In some embodiments, the reference cell is the same cell type as the cytoplast. In some embodiments, the reference cell is an otherwise identical to the cytoplast, except that it does not express ACE2. Viral infectivity can be measured and determined by assays commonly known. Exemplary measurements of viral infectivity can include viral plaque assay, fluorescent focus assay (FFA) and endpoint dilution assay (TCID50). Each of these assays can rely on serial viral dilutions added to cytoplast and/or cell to measure viral infectivity. Other exemplary measurements for determining viral infectivity can include qPCR or ELISA for quantifying the amount of viral genome or particle necessary to infect a set number of cytoplasts and/or cells. In some embodiments, the cytoplast expressing ACE2 can have viral infectivity at least or equal to about 10%, 20%, 30% 40%, 50%, 60%, 70%, 80%, 90%. In some embodiments, the cytoplast expressing ACE2 can have a viral infectivity that at least or equal to about 2 fold, 5 fold, 10 fold, 50 fold, 100 fold, 500 fold, 1000 fold, 5000 fold, or 10000 fold higher than the reference cell.

Described herein, in some embodiments, are cytoplasts engineered to express at least one targeting moiety, such as a homing protein or receptor. In some embodiments, the targeting moiety is secreted by the cytoplast. In some embodiments, the targeting moiety is a ligand for the chemokine receptor described herein. In some embodiments, the targeting moiety is a cytokine described herein. In some embodiments, the targeting moiety is a homing receptor. In some embodiments, the targeting moiety is expressed on the surface of the cytoplast. In some embodiments, the targeting moiety is a chemokine receptor described herein. In some embodiments, the targeting moiety is a receptor for any one of the cytokine described herein.

In some embodiments, the targeting moiety can be specific to one or more ligands expressed on one or more cells in lymph tissue, cells in the lymph tissue can comprise endothelial cells, lymphocytes, macrophages, or reticular cells, or a combination thereof. Non-limiting examples of the secreted targeting moiety include SDF1α, CCL2, CCL3, CCL5, CCL8, CCL1, CXCL9, CXCL10, CCL11, CXCL12, or a combination thereof. In some embodiments, the targeting moiety is expressed on the surface of the cytoplast. Non-limiting examples of the targeting moiety expressed on the surface of the cytoplast include CXCR4, CCR2 or PSGL-1. Non-limiting examples of cell surface proteins that may be expressed on the cell surface include CXCR4, CCR2, CCR1, CCRS, CXCR7, CXCR2, CXCR1, C-X-C chemokine receptor type 3, leukosialin, CD44 antigen, C-C chemokine receptor type 7, L-selectin, lymphocyte function-associated antigen 1, or very late antigen-4, or a combination thereof.

In some embodiments, the cytoplast expressing the targeting moiety (e.g., homing protein or homing receptor) also expresses an active agent disclosed herein. In some embodiments, the active agent is an additional exogenous agent described herein. In some embodiments, the active agent is pathogen-recognized moiety described herein. In some embodiments, the active agent comprises an antibody or single-domain antibody that binds to: an epitope expressed by the pathogen; an epitope associated with a microenvironment associated with the pathogen; or an epitope associated with a biomolecule released by the pathogen. In some embodiments, the binding of the antibody or single-domain antibody to the epitope confers therapeutic or vaccination properties against the pathogen. In some embodiments, the binding of the antibody or single-domain antibody to the epitope recruits immune cells to activate immune response to confer therapeutic properties against the pathogen.

### II. METHODS OF TREATMENT AND PREVETION

Provided herein are methods of treating or preventing a pathogen-associated disease or a condition by administering a cytoplast or pharmaceutical composition containing the cytoplast, of the present disclosure to a subject in need thereof. In some embodiments, the cytoplasts and pharmaceutical compositions thereof are suitable for treatment of a disease or a condition described herein. Such disease or condition may, in some cases, be caused (at least in part) by an infection by a pathogen described herein. In some embodiments, the disease or the condition is cancer, such as for example, caused by an infection by an oncolytic virus.

In some embodiments, methods comprise administering the cytoplast or pharmaceutical composition containing the cytoplast to a subject systemically.

Disclosed herein, in some embodiments, are methods of treating cancer by administering to a subject in need thereof a cytoplast or a pharmaceutical composition containing the cytoplast to the subject. In some embodiments, the cytoplast comprises an exogenous nucleic acid encoding an anti-cancer active agent. In some embodiments, the anti-cancer active agent is a vaccine against an oncolytic virus. In some embodiments, the cytoplast is engineered to express an antibody or small molecule specific to a cancer cell. In some embodiments, the antibody may be a neutralizing antibody may target the cancer cell and subsequently activate the adaptive immune system to neutralize the cancer cell. In some embodiments, the antibody may be a single-domain antibody (e.g., a nanobody). In some embodiments, the antibody may be conjugated to a drug such as a cytotoxic drug to form an antibody drug conjugate (ADC). In some embodiments, the cytoplast confers therapeutic properties by directly contacting the cancer cell. In some embodiments, the cytoplast confers therapeutic properties by recruiting and activating immune response (e.g., immune cells) to the cancer cell.

Also disclosed are methods of vaccinating a subject against a pathogen described herein. In some embodiments, the cytoplast is engineered to express a pathogen antigen for uses as a pathogen vaccine. In some embodiments, the pathogen may be any one of the pathogens selected from Tables 3-6. In some embodiments, the cytoplast is engineered to express an antigen of any one of the pathogens selected from Tables 3-6. In some embodiments, the antigen comprises an amino acid sequence that is at least or equal to about 50%, 60%, 70%, 75%, 80%, 85%, 90%, 95%, or 99% identical to one or more of SEQ ID NOs: 1, 3-7, 151-154, 251-260, 401-447, 551-562, 651-660, 751-761, 851-859, 951-984, 1051-1057, or 1151-1153. In some embodiments, the antigen is encoded from a nucleic acid sequence that is at least or equal to about 50%, 60%, 70%, 75%, 80%, 85%, 90%, 95%, or 99% identical to one or more of SEQ ID NOs: 2, 8, 101-104, 201-209, 301-347, 501-512, 601-610, 701-711, 801-809, 901-934, 1001-1007, or 1101-1103. In some embodiments, the cytoplast is engineered to express a viral antigen for use as a viral vaccine. In some embodiments, the cytoplast is engineered to express a bacterial antigen for use as a bacterial vaccine.

Also described herein are method for treating a subject against a pathogen infection. In some embodiments, the cytoplast is engineered to express an antibody or small molecule specific to a pathogen, that is effective to reduce the pathogen in a subject in need thereof. In some embodiments, the antibody may be a neutralizing antibody that may target the pathogen and subsequently activate the adaptive immune system to neutralize the pathogen. In some embodiments, the antibody can be a single-domain antibody (e.g., a nanobody). In some embodiments, the antibody can be conjugated to a drug such as a cytotoxic drug to form an antibody drug conjugate (ADC). In some embodiments, the cytoplast confers therapeutic properties by directly contacting the pathogen. In some embodiments, the cytoplast confers therapeutic properties by recruiting and activating immune response (e.g., immune cells) to the pathogen.

Disclosed herein, in some embodiments, are methods of treating an infection by a pathogen in a subject, by administering the cytoplast or the pharmaceutical composition containing the cytoplast to the subject, wherein the cytoplast is engineered to trap pathogen in any tissue (e.g., blood, muscle, or lymph) of a subject, prevent propagation of the pathogen in the subject, and optionally, clear the pathogen from the subject, such as for example, by phagocytosis. In some embodiments, the cytoplast is engineered to express a therapeutic agent that is effective to treat the pathogen-associated disease or condition. In some embodiments, the cytoplast is engineered to express a therapeutic agent that is effective to treat cancer. In some embodiments, the method further includes administering to the subject one or more additional therapeutic agents. In some embodiments, the one or more additional therapeutic agents is selected from the group consisting of: cell-based therapy, a small molecule, immuno-therapy, chemotherapy, radiation therapy, gene therapy, and surgery. The additional therapy may be administered to the subject simultaneously with the cytoplasts of the present disclosure. The additional therapy may be administered before or after the cytoplasts of the present disclosure.

A. Disease or Condition

The pathogen-associated disease or condition disclosed herein includes viral infections, bacterial infections, fungal infections, parasitic infections, and protozoal infections, and diseased or condition associated with an infection disclosed herein. In some embodiments, the pathogen may selected from any one of the pathogens listed in Tables 3-6. Non-limiting examples of infections that may be treated or prevented by the compositions and methods utilizing the compositions described herein may include Acinetobacter infections, Actinomycosis, African sleeping sickness (African trypanosomiasis), AIDS (Acquired immunodeficiency syndrome), Amebiasis, Anaplasmosis, Angiostrongyliasis, Anisakiasis, Anthrax, Arcanobacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas Disease (American trypanosomiasis), Chancroid, Chickenpox, Chikungunya, Chlamydia, Chlamydophila pneumoniae c infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus infection, Creutzfeldt—Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human immnunodeficiency virus (HIV) infection, Human metapneumovirus infection, Human monocytic ehrlichiosis, Human papillomavirus (HPV) infection, Human parainfluenza virus infection, Hymenolepiasis, Epstein—Barr virus infectious mononucleosis (Mono), Influenza (flu), influenza virus A, influenza virus B, influenza virus C, influenza virus D, influenza virus pr8, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma (disambiguation), Myiasis, Neonatal conjunctivitis (Ophthalmia neonatorum), Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease (PID), Pertussis (Whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Respiratory Syncytial virus (RSV), Rubella, Salmonellosis, SARS (Severe Acute Respiratory Syndrome), Scabies, Scarlet fever, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Subacute sclerosing panencephalitis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans (OLM)), Toxocariasis (Visceral Larva Migrans (VLM)), Toxoplasmosis, Trachoma, Trichinosis, Trichomoniasis, Trichuriasis (Whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile Fever, White piedra (Tinea blanca), Yersinia pseudotuberculosis infection, Yersiniosis, Yellow fever, Zika fever, and Zygomycosis.

The coronavirus infection may be an infection by an alpha coronavirus or a beta coronavirus. Non-limiting examples of alpha coronavirus include 229E and NL63. Non-limiting examples of beta coronavirus include OC43, HKU1, severe acute respiratory syndrome (SARS) coronavirus, or Middle East Respiratory Syndrome (MERS) coronavirus. In some embodiments, the SARS coronavirus is SARS-CoV, SARS-CoV-2, or a variant thereof. In some embodiments, the MERS coronavirus is MERS-CoV or a variant thereof. In some embodiments, the SARS coronavirus causes a disease or a condition, such as coronavirus disease 2019 (COVID-19).

The coronavirus described herein, in some embodiments, is encoded by a nucleic acid sequence provided in any one of SEQ ID NOs: 1 and 3-7. In some embodiments, the coronavirus (or variant thereof) is encoded by a nucleic acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 1 and 3-7.

In some embodiments, the coronavirus comprises a spike protein encoded an amino acid sequence provided in SEQ ID NO: 2 or 8. In some embodiments, the S protein is encoded by an amino acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2 or 8.

In some embodiments, the coronavirus comprises a nucleocapsid (N) protein encoded by an amino acid sequence provided in SEQ ID NO: 9. In some embodiments, the N protein is encoded by an amino acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 9.

In some embodiments, the coronavirus comprises a membrane (M) protein encoded by an amino acid sequence provided in SEQ ID NO: 10. In some embodiments, the M protein is encoded by an amino acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 10.

In some embodiments, the coronavirus comprises an envelope (E) protein encoded by an amino acid sequence provided in SEQ ID NO: 11. In some embodiments, the E protein is encoded by an amino acid sequence that is at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 11.

B. Subject

In some embodiments, the subject is in need of, has been determined to be in need of, or is suspected to be in need of a treatment. As used herein, the term “subject” refers to any organism. For example, a subject can be a mammal, amphibian, fish, reptile, invertebrate, bird, plant, archaea, fungus, or bacteria. In some embodiments, the subject is a mammal. In some embodiments, the subject may be a rodent (e.g., a mouse, a rat, a hamster, a guinea pig), a canine (e.g., a dog), a feline (e.g., a cat), an equine (e.g., a horse), an ovine, a bovine, a porcine, a non-human primate, e.g., a simian (e.g., a monkey), an ape (e.g., a gorilla, a chimpanzee, an orangutan, a gibbon), or a human. In some embodiments of any of the methods described herein, the subject is between 0 and 120 years old (e.g., between birth and one month (e.g., a neonate), between one month and two years (e.g., an infant), between 2 years and 12 years (e.g., a child), between twelve years and sixteen years (e.g., an adolescent), between 1 and 120 years old, between 1 and 115 years old, between 1 and 110 years old, between 1 and 105 years old, between 1 and 100 years old, between 1 and 95 years old, between 1 and 90 years old between 1 and 85 years old, between 1 and 80 years old, between 1 and 75 years old, between 1 and 70 years old, between 1 and 65 years old, between 1 and 60 years old, between 1 and 50 years old, between 1 and 40 years old, between 1 and 30 years old, between 1 and 25 years old, between 1 and 20 years old, between 1 and 15 years old, between 1 and 10 years old, between 5 and 120 years old, between 5 and 110 years old, between 5 and 100 years old, between 5 and 90 years old, between 5 and 60 years old, between 5 and 50 years old, between 5 and 40 years old, between 5 and 30 years old, between 5 and 20 years old, between 5 and 10 years old, between 10 and 120 years old, between 10 and 110 years old, between 10 and 100 years old, between 10 and 90 years old, between 10 and 80 years old between 10 and 60 years old, between 10 and 50 years old, between 10 and 40 years old, between 10 and 30 years old, between 10 and 20 years, between 20 and 120 years old, between 20 and 110 years old, between 20 and 100 years old, between 20 and 90 years old, between 20 and 70 years old, between 20 and 60 years old, between 20 and 50 years old, between 20 and 40 years old, between 20 and 30 years old, between 30 and 120 years old, between 30 and 110 years old, between 30 and 100 years old, between 30 and 90 years old, between 30 and 70 years old, between 30 and 60 years, between 30 and 50 years old, between 40 and 120 years old, between 40 and 110 years old, between 40 and 100 years old, between 40 and 90 years old, between 40 and 80 years old, between 40 and 60 years old, between 40 and 50 years old, between 50 and 120 years old, between 50 and 110 years old, between 50 and 100 years old, between 50 and 90 years old, between 50 and 80 years old, between 50 and 70 years old, between 50 and 60 years old, between 60 and 120 years old, between 60 and 110 years old, between 60 and 100 years old, between 60 and 90 years old, between 60 and 80 years old, between 60 and 70 years old, between 70 and 120 years old, between 70 and 110 years old, between 70 and 100 years old, between 70 and 90 years old, between 70 and 80 years old, between 80 and 120 years old, between 80 and 110 years old, between 80 and 100 years old, between 80 and 90 years old, between 90 and 120 years old, between 90 and 110 years old, between 90 and 100 years old, between 100 and 120 years old, or between 110 and 120 years old). In some embodiments of any of the methods described herein, the subject is not yet born, e.g., in utero. In some embodiments of any of the methods described herein, the subject is at least 1 month old (e.g., at least 2 years old, at least 12 years old, at least 16 years old, or at least 18 years old). Any of the methods described herein can be used to treat a subject, e.g., a diseased subject (i.e., a subject with a disease, e.g., who has been diagnosed with a disease), or an asymptomatic subject (i.e., a subject who clinically presents as healthy, or who has not been diagnosed with a disease). As used herein, treating includes “prophylactic treatment” which means reducing the incidence of or preventing (or reducing risk of) a sign or symptom of a disease in a subject at risk for the disease, and “therapeutic treatment”, which means reducing signs or symptoms of a disease, reducing progression of a disease, reducing severity of a disease, re-occurrence in a subject diagnosed with the disease. As used herein, the term “treat” means to ameliorate at least one clinical parameter of the disease, and/or to provide benefits (e.g., anti-aging, anti-scarring, wound healing, anti-depressant, anti-inflammatory, weight loss).

C. Dosing Frequency and Administration

In some embodiments of any of the methods provided herein, the composition is administered at least once (e.g., 2, 3, 4, 5, 6,7, 8, 9, 10, 11, 12, 13, 14, 15, 16, 17, 18, 19, 20, 30, 40, 50, 60, 70, 80, 90, 100 times) during a period of time (e.g., every day, every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). Also contemplated are monthly treatments, e.g., administering at least once per month for at least 1 month (e.g., at least two, at least three, at least four, at least five, at least six or more months, e.g., 12 or more months), and yearly treatments (e.g., administration once a year for one or more years). The frequency of the administration may be relative to a particular event, such as for example, a first symptom of a pathogen-associated disease or disorder, a first dose of a vaccine composition, travel to a another state, county, country, or continent, and so forth.

Administration can be via any suitable route, e.g., subcutaneous, intravenous, arterial, ocular, oral, intramuscular, intranasal (e.g., inhalation), intraperitoneal, topical, mucosal, epidural, sublingual, epicutaneous, extra-amniotic, inter-articular, intradermal, intraosseous, intrathecal, intrauterine, intravaginal, intravesical, intravitreal, perivascular, and/or rectal administration, or any combination of known administration methods.

In some embodiments, the death process of cytoplasts can have a therapeutic effect on a subject. For example, in some embodiments, the death process of cytoplasts can be immunostimulatory. Accordingly, provided herein are methods of administering cytoplasts to a subject, wherein the death of the cytoplasts has a therapeutic effect on the subject. In some embodiments, the cytoplasts administered to the subject are dead. In some embodiments, the cytoplasts administered to the subject, when administered, have a remaining life span of less than 5 days (e.g., less than 4 days, less than 3 days, less than 2 days, less than 36 hours, less than 1 day, less than 18 hours, less than 12 hours, less than 6 hours, less than 2 hours, or less than 1 hour).

In some embodiments, cells can be removed from a subject and enucleated. In some embodiments, the cells are engineered (e.g., to produce or contain a therapeutic DNA molecule, a therapeutic RNA molecule, a therapeutic protein, a therapeutic peptide, a small molecule therapeutic, a therapeutic gene-editing factor a therapeutic nanoparticle and/or another therapeutic agent) before being enucleated. In some embodiments, cells from a subject are enucleated, and then engineered (e.g., to produce or contain a therapeutic DNA molecule, a therapeutic RNA molecule, a therapeutic protein, a therapeutic peptide, a small molecule therapeutic, a therapeutic gene-editing factor a therapeutic nanoparticle and/or another therapeutic agent). In some embodiments, the cytoplasts (whether or not they have been engineered) are administered to the subject from which the cells were removed.

In some embodiments, the media in which the cytoplasts were cultured and/or stored (a “conditioned media”) can have a therapeutic benefit. In some embodiments, the media in which cytoplasts were co-cultured and/or stored (e.g., after enucleation) with cells (a “conditioned media”) can have a therapeutic benefit. In some embodiments, the media in which cytoplasts fused with cells were cultured and/or stored with cells (a “conditioned media”) can have a therapeutic benefit.

Accordingly, provided herein are methods of treating, preventing, or prophylactically treating, or promoting health in a subject comprising administering to the subject conditioned media. Without being bound by any particular theory, it is believed that, in some embodiments, the therapeutic benefit of cultured media can be due to the presence in the media of exosomes (e.g., containing therapeutic protein) secreted by the cytoplasts.

In some embodiments of any of the methods provided herein, the composition is administered with one or more additional therapies (e.g., any drug (e.g., antibiotics, antivirals, anti-inflammatory medications) or chemotherapy (e.g., a chemotherapeutic agent (e.g., doxorubicin, paclitaxel, cyclophosphamide), or any of the small molecule therapeutics described herein), cell-based therapy, radiation therapy, immunotherapy, a small molecule, an inhibitory nucleic acid (e.g., antisense RNA, antisense DNA, miRNA, siRNA, lncRNA), an exosome-based therapy, gene therapy or surgery). In some embodiments, the one or more additional therapies comprise combination therapy inhibiting an immune checkpoint protein such as PD-1/PDCD1/CD279, CTLA-4/CD152, TIM-3/HAVCR2, TIGIT, LAG3, VISTA/C10orf54, BTLA/CD272, A2AR, KIR, CD28, ICOS/CD278, CD40L/CD154, CD137/4-1BB, CD27, OX40/CD134/TNFRSF4, GITR, or SIRPα.

In some embodiments provided herein, the composition further includes one or more additional therapies (e.g., any drug (e.g., antibiotics, antivirals) or chemotherapy (e.g., a chemotherapeutic agent (e.g., doxorubicin, paclitaxel, cyclophosphamide)), cell-based therapy, radiation therapy, immunotherapy, a small molecule, an inhibitory nucleic acid (e.g., antisense RNA, antisense DNA, miRNA, siRNA, lncRNA) or surgery).

### III. METHODS OF MANUFACTURING

The present disclosure provides methods of manufacturing the anti-viral compositions and cytoplasts disclosed herein. In some embodiments, the disclosure provides methods for the removal of the cell nucleus (also called enucleation) from any nucleated cell derived (e.g., obtained) from either normal or cancer cell lines or any primary cell removed from the body including, but not limited to, commonly used therapeutic cells derived (e.g., obtained) from the immune system (e.g., natural killer (NK) cells, neutrophils, macrophages, lymphocytes, mast cells, basophils, eosinophils), stem cells (including, for example, iPSC (induced pluripotent stem cells), adult stem cells (e.g., mesenchymal stem cells), and embryonic stem cells), and fibroblasts. Cell enucleation can create a therapeutic cytoplast which is viable for a limited period of time, for example, up to 5 days. Therefore, the present disclosure, in some aspects, provides a new use for cytoplasts as a safe therapeutic vehicle that cannot perform one or more of the following actions: proliferate, differentiate, permanently engraft into the subject, become cancerous, or transfer nuclear-encoded DNA/genes to the subject (e.g., transfer of dangerous nuclear-encoded DNA/genes to the subject).

For cell-based therapies, FDA approval has, in some cases, rested on the evidence that cells are stable, meaning that they do not change or become dangerous once inside a subject. However, current cell products, including primary cells, irradiated cells, or “death-switch” controlled cells, still have the potential to respond to or change in the in vivo microenvironment. Importantly, current therapies can still retain the potential to transcribe new genes, which is not a controllable response in vivo. This gene transcription hampers the ability to satisfy regulatory requirements. In contrast, cytoplasts, which lack a nucleus, generally do not have the potential for new gene transcription even in very different in vivo microenvironments, and therefore are a more controlled and safer cell-based therapy.

To date, cell-based therapeutics generally use normal or engineered nucleated cells. Some cell-based therapies irradiate cells prior to subject administration in order to prevent cell proliferation and induced lethal DNA-damage. However, this approach induces mutations and produces significant amounts of reactive oxygen species that can irreversibly damage cellular proteins and DNA, which can release large amounts of damaged/mutated DNA into the body of a subject. Such products can be dangerous if they integrate into other cells and/or induce an unwanted anti-DNA immune response. Irradiated cells can also be dangerous because they can transfer their mutated DNA and genes to host cells by cell-cell fusion. Removing the entire nucleus from a cell is a less damaging and significantly safer method for limiting cellular lifespan that can preclude any introduction of nuclear DNA into a subject. Furthermore, many stem cells, such as mesenchymal stem cells (MSCs), are highly resistant to radiation-induced death, and therefore cannot be rendered safe using this method. In other cases, therapeutic cells have been engineered with a drug-inducible suicide switch to limit cellular lifespan. However, activation of the switch in vivo can require administering a subject with potent and potentially harmful drugs with unwanted side effects. While this method can induce suicide in culture cells (e.g., greater than 95%), it is expected to be inefficient when translated into the clinic. Without being bound by any particular theory, it is believed that a drug-inducible suicide switch could be an insufficient safety measure for clinical practice, since not all cells in the subject may undergo drug-induced death. Therefore, in the case of extensively engineered cells or stem cells or cancer cells, a drug-induced suicide switch could be considered dangerous or insufficient for clinical practice. Moreover, the death of a therapeutic cell can release large amounts of DNA (normal or genetically altered), which can integrate into host cells or induce a dangerous systemic anti-DNA immune response. If the cell mutates and/or loses or inactivates the suicide switch, it can become an uncontrollable mutant cell. In addition, these cells can fuse with host cells in the subject, and therefore transfer DNA (e.g., mutant DNA). Such fused cells can be dangerous because not all host cells inherit the suicide gene, but can inherit some of the therapeutic cell's genes/DNA during chromosomal reorganization and cell hybridization. In addition, for the same reason, therapeutic cells with suicide switches may not be ideal for use as cell fusion partners in vitro. Another method to limit therapeutic cell lifespan is heat-induced death that causes severe damage that terminates biological functions beneficial in therapeutic use (e.g., protein translation). Unlike cytoplasts, nucleated cell therapies and even some cells inactivated by the methods described above can still transfer DNA to the subject since they retain their nucleus and genetic material. Numerous chemicals inhibit cell proliferation and/or cause cell death prior to therapeutic use, including chemotherapeutic drugs and mitomycin C, etc. However, such drugs can have significant off-target effects that significantly damage the cell, which are unwanted for clinical applications due to high toxicities. Many anti-proliferative and death-inducing drugs do not effectively inhibit 100% of the cells due to resistance, and unlike cytoplasts, many drug effects are reversible. Thus, this approach is not suitable to prevent cell growth of immortalized or cancer cells in vivo.

Provided herein are methods of manufacturing a cytoplast of the present disclosure. In some embodiments, the nucleate cell (e.g., referred to herein as a “parent cell”) is treated with cytochalasin B to soften the cortical actin cytoskeleton. In some embodiments, methods comprise introducing an active agent such as viral peptide or protein, to a nucleated cell; and mechanically removing the nucleus from the parent cell to produce a cytoplast (enucleation). In some embodiments, the parent cell is also introduced to a second active agent prior to enucleation. In some embodiments the parent cell is introduced to the second active agent after enucleation. The second active agent may be a therapeutic agent that is delivered by the cytoplast to the target cell. An exemplary target cell is a muscle cell, such as a myoblast or a mature muscle cell.

The active agent is introduced to the parent cell using a suitable transient transfection methods (e.g., electroporation) or transduction (e.g., viral-mediated). In some embodiments, a plasmid comprising a transgene encoding the active agent is transfected into the parent cell. In some embodiments, a viral vector comprising a transgene encoding the active agent is transduced into the parent cell. The plasmid can be a bacterial plasmid (e.g., E.coli). In some embodiments, the parent cell is also introduced to a second active agent by a similar method. In some embodiments, the second active agent is a therapeutic agent.

The nucleus of the parent cell expressing the active agent, and optionally, the second active agent, is removed using mechanical enucleation. In some embodiments, the parent cell wall is permeabilized using a cell-permeable mycotoxin. Mechanical enucleation may include performing a density gradient centrifugation using discontinuous Ficoll gradients, high-speed centrifugation, to form a cytoplast. The cytoplast is isolated and purified using standard purification protocols. The cytoplast may be further engineered with an exogenous nucleic acid (e.g., mRNA, DNA, antisense oligonucleotide).

The present disclosure provides methods for manufacturing cytoplasts with either natural or inducible expression and/or uptake of biomolecules with therapeutic functions including, but not limited to, DNA/genes (e.g., plasmids) RNA (e.g., mRNA, shRNA, siRNA, miRNA), proteins, peptides, small molecule therapeutics (e.g., small molecule drugs), gene editing components, nanoparticles, and other therapeutic agents (e.g., bacteria, bacterial spores, bacteriophages, bacterial components, viruses (e.g., oncolytic viruses), exosomes, lipids, or ions).

Various methods are known in the art that can be used to introduce a biomolecule (e.g., a RNA molecule (e.g., mRNA, miRNA, siRNA, shRNA, lncRNA), a DNA molecule (e.g., a plasmid), a protein, a gene-editing factor (e.g., a CRISPR/Cas9 gene-editing factor), a peptide, a plasmid) into a cytoplast (e.g., a cytoplast derived from any cell described herein). Non-limiting examples of methods that can be used to introduce a biomolecule into a cytoplast include: electroporation, microinjection, lipofection, transfection, calcium phosphate transfection, dendrimer-based transfection, cationic polymer transfection, cell squeezing, sonoporation, optical transfection, impalection, hydrodynamic delivery, magnetofection, and nanoparticle transfection. Non-limiting examples of gene-editing factors include: CRISPR/Cas9 gene-editing, transcription activator-like effector nuclease (TALEN), and zinc finger nucleases.

Methods of culturing a cell (e.g., any of the cells described herein) are well known in the art. Cells can be maintained in vitro under conditions that favor growth, proliferation, viability, differentiation and/or induction of specific biological functions with therapeutic capabilities/benefits including, but not limited to, 3-dimensional culturing, hypoxic environments, culturing on defined extracellular matrix components, treatment with chemical agents, cytokines, growth factors or exposure to any exogenous agent natural or synthetic that induces a specific desirable cell response.

Methods encompass the largescale in vitro production of cytoplasts derived (e.g., obtained) from any nucleated cell type (e.g., a mammalian cell, a human cell), a protozoal cell (e.g., an amoeba cell), an algal cell, a plant cell, a fungal cell, an invertebrate cell, a fish cell, an amphibian cell, a reptile cell, or a bird cell). For example, the cell can have been immortalized and/or oncogenically transformed naturally or by genetic engineering.

Provided herein methods of storing the purified and isolated cytoplasts of the present disclosure such that the biological activity of the cytoplast is slowed or stopped completely. In some embodiments, the cytoplast is stored in a suspension animation at a temperature that is at most 10° C. In some embodiments, the temperature is about 4° C. In some embodiments, the temperature is 4° C. In some embodiments, the temperature is at most 4° C. In some embodiments, the cytoplast is stored for at most or about 96 hours. After a period of time, the cytoplast is removed from the suspension animation to revive the biological activity of the cytoplast. The resulting cytoplast is viable, and suitable for delivery to a subject in need thereof. In some embodiments, the cytoplasts stored at between 4° C. to 10° C. exhibit at least or equal to 10%, 20%, 30%, 40%, 50%, 60%, 70%, 75%, 80%, 85%, 90%, 95%, or 99% viability as compared with a cytoplast prior to being stored at between 4° C. to 10° C.

In some embodiments of any of the compositions and methods provided herein, the cytoplast is cooled or frozen for later use. Various methods of preserving cells are known in the art, including, but not limited to, the use of a serum (e.g., Fetal Bovine Serum) and dimethyl sulfoxide (DMSO) at ultralow temperatures (frozen cryopreservation) or hibernation media for storage at 4° C. (cryohibernation). In some embodiments of any of the compositions and methods provided herein, the cytoplast is thawed prior to use.

In some embodiments, the cytoplasts can be stored at a temperature between about −80° C. and about 16° C. (e.g., about −80° C. and about 12° C., −80° C. and about 10° C., about −80° C. and about 8° C., about −80° C. and about 6° C., about −80° C. and about 4° C., about −80° C. and about 2° C., about −80° C. and about 0° C., about −80° C. and about −4° C., about −80° C. and about −10° C., about −80° C. and about −16° C., about −80° C. and about −20° C., about −80° C. and about −25° C., about −80° C. and about −30° C., about −80° C. and about −35° C., about −80° C. and about −40° C., about −80° C. and about −45° C., about −80° C. and about −50° C., about −80° C. and about −55° C., about −80° C. and about −60° C., about −80° C. and about −65° C., about −80° C. and about −70° C., about −60° C. and about 16° C., about −60° C. and about 12° C., about −60° C. and about 10° C., about −60° C. and about 8° C., about −60° C. and about 6° C., about −60° C. and about 4° C., about −60° C. and about 2° C., about −60° C. and about 0° C., about −60° C. and about −4° C., about −60° C. and about −10° C., about −60° C. and about −10° C., about −60° C. and about −16° C., about −60° C. and about −20° C., about −60° C. and about −25° C., about −60° C. and about −30° C., about −60° C. and about −35° C., about −60° C. and about −40° C., about −60° C. and about −50° C., about −50° C. and about 16° C., about −50° C. and about 12° C., about −50° C. and about 10° C., about −50° C. and about 8° C., about −50° C. and about 6° C., about −50° C. and about 4° C., about −50° C. and about 2° C., about −50° C. and about 0° C., about −50° C. and about −4° C., about −50° C. and about −10° C., about −50° C. and about −16° C., about −50° C. and about −20° C., about −50° C. and about −30° C., about −50° C. and about −40° C., about −20° C. and about 16° C., about −20° C. and about 12° C., about −20° C. and about 10° C., about −20° C. and about 8° C., about −20° C. and about 6° C., about −20° C. and about 4° C., about −20° C. and about 2° C., -about 20° C. and about 0° C., about −20° C. and about −4° C., about −20° C. and about −10° C., about −20° C. and about −15° C., about −10° C. and about 16° C., about −10° C. and about 12° C., about −10° C. and about 10° C., about −10° C. and about 8° C., about −10° C. and about 6° C., about −10° C. and about 4° C., about −10° C. and about 2° C., about −10° C. and about 0° C., about −10° C. and about −4° C., about −10° C. and about −6° C., about −4° C. and about 16° C., about −4° C. and about 10° C., about −4° C. and about 6° C., about −4° C. and about 4° C., about −4° C. and about 2° C., about −4° C. and about 0° C., about −2° C. and about 16° C., about −2° C. and about 12° C., about −2° C. and about 10° C., about −2° C. and about 6° C., about −2° C. and about 4° C., about −2° C. and about 2° C., about −2° C. and about 0° C., about 0° C. and about 16° C., about 0° C. and about 14° C., about 0° C. and about 12° C., about 0° C. and about 10° C., about 0° C. and about 8° C., about 0° C. and about 6° C., about 0° C. and about 4° C., about 2° C. and about 16° C., about 2° C. and about 12° C., about 2° C. and about 10° C., about 2° C. and about 8° C., about 2° C. and about 6° C., about 2° C. and about 4° C., about 4° C. and about 16° C., about 4° C. and about 12° C., about 4° C. and about 10° C., about 4° C. and about 8° C., about 4° C. and about 6° C., about 6° C. and about 16° C., about 6° C. and about 12° C., about 6° C. and about 10° C., about 6° C. and about 8° C., about 8° C. and about 16° C., about 8° C. and about 12° C., about 8° C. and about 10° C., about 10° C. and about 16° C., about 10° C. and about 12° C., or about 12° C. and about 16° C.) for about 1 day to about 7 days (e.g., about 1 day to about 6 days, about 1 day to about 5 days, about 1 day to about 4 days, about 1 day to about 3 days, about 1 day to about 2 days, about 2 days to about 7 days, about 2 days to about 6 days, about 2 days to about 5 days, about 2 days to about 4 days, about 2 days to about 3 days, about 3 days to about 7 days, about 3 days to about 6 days, about 3 days to about 5 days, about 3 days to about 4 days, about 4 days to about 7 days, about 4 days to about 6 days, about 4 days to about 5 days, about 5 days to about 7 days, about 5 days to about 6 days, or about 6 days to about 7 days). In some embodiments, the cytoplasts stored at the temperature ranges described herein exhibit at least or equal to 10%, 20%, 30%, 40%, 50%, 60%, 70%, 75%, 80%, 85%, 90%, 95%, or 99% viability as compared with a cytoplast prior to being stored at the same temperature ranges.

In some embodiments, the cytoplasts are lyophilized. In some embodiments, the cytoplasts are lyophilized for storage. In some embodiments, the cytoplasts are lyophilized for at least or equal to 1 hour, 2 hours, 4 hours, 6 hours, 8 hours, 12 hours, 24 hours, 2 days, 3 days, 4 days, 5 days, 6 days, 7 days, 8 days, 9 days, 10 days, 12 days, 14 days, 16 days, 18 days, 20 days, 22 days, 24 days, 26 days, 28 days, 30 days, 2 months, 3 months, 4 months, 5 months, 6 months, 2 months, 3 months, 4 months, 5 months, 6 months, 7 months, 8 months, 9 months, 10 months, 11 months, 12 months, 18 months, 24 months, 30 months, 3 years, 4 years, 5 years, or 10 years. In some embodiments, the cytoplasts exhibit at least or equal to 10%, 20%, 30%, 40%, 50%, 60%, 70%, 75%, 80%, 85%, 90%, 95%, or 99% viability as compared with a cytoplast prior to being lyophilized.

### IV. KITS

Disclosed herein, in some embodiments, are kits for using the compositions, the pharmaceutical compositions, or the cytoplasts described herein. In some embodiments, the kits disclosed herein may be used to prevent or treat a disease or condition in a subject; or select a subject for prevention or treatment for the disease or condition disclosed herein. In some embodiments, the kit comprises the pharmaceutical compositions, the compositions, or the cytoplasts described herein, which may be used to perform the methods described herein. Kits comprise an assemblage of materials or components. Thus, in some embodiments the kit contains a composition including of the pharmaceutical composition or the cytoplast, for the treatment of the disease or disorder described herein.

In some embodiments, the kit described herein comprises components for selecting for a homogenous population of the cytoplasts. In some embodiments, the kit described herein comprises components for selecting for a heterogenous population of the cytoplasts. In some embodiments, the kit comprises the components for assaying the number of units of the exogenous therapeutic synthesized or released by the cytoplast. In some embodiments, the kit comprises the components for assaying the number of units of the exogenous therapeutic expressed on the surface of the cytoplast. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of vaccinating or treating a disease or condition disclosed herein (e.g., respiratory disease) in a subject. In some embodiments, the kit is configured particularly for the purpose of vaccinating or treating mammalian subjects. In some embodiments, the kit is configured particularly for the purpose of vaccinating or treating human subjects.

Instructions for use may be included in the kit. For the example, the instruction may direct healthcare providers how to vaccinate the subject with the components of the kit in a medical facility or in a point of care capacity. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example the components may be in dissolved, dehydrated, or lyophilized form; they may be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package may be a glass vial or prefilled syringes used to contain suitable quantities of the pharmaceutical composition. The packaging material has an external label which indicates the contents and/or purpose of the kit and its components.

### V. DEFINITIOINS

Unless defined otherwise, all terms of art, notations and other technical and scientific terms or terminology used herein are intended to have the same meaning as is commonly understood by one of ordinary skill in the art to which the claimed subject matter pertains. In some embodiments, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Any systems, methods, software, compositions, and platforms described herein are modular and not limited to sequential steps. Accordingly, terms such as “first” and “second” do not necessarily imply priority, order of importance, or order of acts.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

As used herein, a “cell” generally refers to a biological unit of a living organism.

As used herein, the term “eukaryotic cell” refers to a cell having a distinct, membrane-bound nucleus. Such cells may include, for example, mammalian (e.g., rodent, non-human primate, or human), non-mammalian animal (e.g., fish, bird, reptile, or amphibian), invertebrate, insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells.

As used herein, the term “cytoplast,” “cell without a nucleus,” or “enucleated cell” are used interchangeably to refer to a nucleus-free cell that was obtained from a previously nucleated cell (e.g., any cell described herein). In some embodiments, the nucleated cell comprises cell organelles and the cytoplast derived from the nucleated cell retains such organelles, which in some cases, enables cellular functions such as cell motility, protein synthesis, protein secretion, and the like. In some embodiments “obtaining” does not involve differentiating the nucleated cell into an enucleated cell using natural processes or otherwise.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif.; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The terms “transfection” or “transfected” generally refers to introduction of a nucleic acid into a cell by non-viral or viral-based methods. The nucleic acid molecules can be gene sequences encoding complete proteins or functional portions thereof. See, e.g., Sambrook et al., 1989, Molecular Cloning: A Laboratory Manual, 18.1-18.88.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which can be located upstream or downstream of the coding sequence. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” can encompass mRNA, cDNA and genomic forms of a gene.

The term “mutation,” as used herein, can refer to a substitution of a residue within a sequence, e.g., a nucleic acid or amino acid sequence, with another residue, or a deletion or insertion of one or more residues within a sequence. One or more mutations can be described by identifying the original residue followed by the position of the residue within the sequence and by the identity of the newly substituted residue. Mutation can be a change or alteration in a sequence (e.g., nucleic acid sequence, genomic sequence, genetic sequence such as DNA, RNA, or protein sequence) relative to a reference sequence. The reference sequence can be a wild-type sequence, a sequence of a healthy or normal cell, or a sequence that is not associated with a disease or a disorder. A reference sequence can be a sequence not associated with a cancer. Non-limiting examples of mutations include point mutations, substitution of one or more nucleotides, deletion of one or more nucleotides, insertion of one or more nucleotides, fusion of one or more nucleotides, frame shift mutation, aberration, alternative splicing, abnormal methylation, missense mutation, conservative mutation, non-conservative mutation, nonsense mutation, splice variant, alternative splice variant, transition, transversion, de novo mutation, deleterious mutation, disease-causing mutation, epimutation, founder mutation, germline mutation, somatic mutation, predisposing mutation, splice-site mutation, or susceptibility gene mutation. The mutation can be a pathogenic variant or mutation that increases an individual's susceptibility or predisposition to a certain disease or disorder. The mutation can be a driver mutation (e.g., a mutation that can confer a fitness advantage to cells in their microenvironment, thereby driving the cell lineage to cancer). The driver mutation can be a lost function mutation. The mutation can be a lost function mutation. The mutation can be a passenger mutation (e.g., a mutation that occurs in a genome with the driver mutation and can be associated with clonal expansion). As used herein, the term “gene” can refer to a combination of polynucleotide elements, that when operatively linked in either a native or recombinant manner, provide some product or function.

As used herein, the terms “polypeptide,” “peptide” and “protein” can be used interchangeably herein in reference to a polymer of amino acid residues. A protein can refer to a full-length polypeptide as translated from a coding open reading frame, or as processed to its mature form, while a polypeptide or peptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the terms “portion,” or “fragment,” or equivalent terms can refer to a portion of an entity (e.g., a protein). In the case of proteins or polypeptides, a portion or fragment is less than the full-length of the protein or polypeptide. In some embodiments, the portion or fragment maintains an intended function of the full-length protein.

The terms “complement,” “complements,” “complementary,” and “complementarity,” as used herein, generally refer to a sequence that is fully complementary to and hybridizable to the given sequence. In some embodiments, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g. the EMBOSS Needle aligner available at www.ebi.ac.uk/Tools/psa/emboss_needle/nucleotide.html, optionally with default settings), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject” and “individual,” are often used interchangeably herein, to refer to a biological entity containing expressed genetic materials. As used herein, the term “subject” refers to any organism. For example, a subject can be a mammal, amphibian, fish, reptile, invertebrate, bird, plant, archaea, fungus, or bacteria. In some embodiments, the subject is a mammal. In some embodiments, the subject may be a rodent (e.g., a mouse, a rat, a hamster, a guinea pig), a canine (e.g., a dog), a feline (e.g., a cat), an equine (e.g., a horse), an ovine, a bovine, a porcine, a non-human primate, e.g., a simian (e.g., a monkey), an ape (e.g., a gorilla, a chimpanzee, an orangutan, a gibbon), or a human. The subject can be tissues, cells and their progeny of a biological entity obtained in vivo or cultured in vitro. The subject can be a mammal. The mammal can be a human. The subject may be a “patient,” which in some embodiments, refers to a subject that has been diagnosed or has a disease or condition described herein. In some embodiments, the subject has not been diagnosed, but is predicted to beat high risk for developing or having the disease or the condition.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “adaptive immune response” as used herein refers to the components of the immune response that respond in an antigen-restricted way and encompasses cellular immune responses attributable to T lymphocytes and humoral or antibody response attributable to B cells and plasma cells. A “cellular immune response” is indicated by any one or more of the following: cytokine/chemokine release by T cells; T-cell homing to secondary lymphoid organs; T-cell proliferation; and cytotoxic T-cell responses. Several methods can be used to verify an antigen-specific cellular immune response, including ex vivo antigen stimulation assays of T lymphocytes and in vivo assays, such as tetramer staining of T lymphocytes. An “antibody response” is indicated by any one or more of the following: B cell proliferation, B-cell cytokine/chemokine release, B-cell homing to secondary lymphoid organs, antibody secretion, isotype switching to IgG type antibodies, or plasma cell differentiation. An antibody response can be verified by several methods, but a predominant method is the detection of antigen-specific antibodies in the serum or plasma of a vaccinated individual.

An “adjuvant” as described herein refers to a substance that in combination with an antigen promotes an adaptive immune response to the antigen. An “immune stimulatory compound” refers to a substance that specifically interacts with the innate immune system to initiate a “danger signal” that ultimately leads to the development of the adaptive components of the immune response (e.g., B cell, T cells). Immune stimulatory compounds include pathogen-associated molecular patterns (PAMPs) such as dsRNA, lipopolysaccharide, and CpG DNA, either naturally occurring or synthetic. Immune stimulatory compounds are agonists of various innate immune receptors including Toll-like receptors (TLRs), NOD-like receptors, RIG-1 or MDA-5 receptors, C-type lectin receptors, or the STING pathway.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition”; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration. The section headings used herein are for organizational purposes only and are not to be construed as limiting the subject matter described.

### VI. EMBODIMENTS

### Compositions

Disclosed herein are compositions in accordance with the embodiments below:

Embodiment 1. A composition comprising a cell that is enucleated and comprises an anti-viral agent.

Embodiment 2. The composition of embodiment 1, wherein the anti-viral agent is an attenuated version of a viral antigen, a virus, or an antibody specific to the viral antigen.

Embodiment 3. The composition of embodiment 2, wherein the viral antigen is a viral protein, peptide fragment, nucleic acid, or sugar moiety, and wherein the antibody specific to the viral antigen is specific to the viral protein, peptide fragment, nucleic acid, or sugar moiety.

Embodiment 4. The composition of embodiment 2, wherein the cell comprises one or more intracellular organelles for in vivo protein synthesis or protein secretion of the anti-viral agent.

Embodiment 5. The composition of embodiment 4, wherein the one or more intracellular organelles is selected from Golgi apparatus, ribosome, endoplasmic reticulum.

Embodiment 6. The composition of any previous embodiment, wherein the cell has a diameter of about 1 micrometers to 100 micrometers in length.

Embodiment 7. The composition of any previous embodiment, wherein the cell is a stem cell.

Embodiment 8. The composition of embodiment 7, wherein the stem cell is a mesenchymal stem cell or an induced pluripotent stem cell.

Embodiment 9. The composition of embodiment 8, wherein the mesenchymal stem cell is from adipose tissue or bone.

Embodiment 10. The composition of embodiment 8, wherein the induced pluripotent stem cell is from urine, saliva, hair, skin, or feces.

Embodiment 11. The composition of embodiments 2-10, wherein the viral antigen or the antibody specific to the viral antigen is expressed at a surface of the cell or is secretory.

Embodiment 12. The composition of any previous embodiment, wherein the viral antigen of the virus is tethered to a surface of the cell by a linker selected from a chemical linker, peptide linker, or a polymer.

Embodiment 13. The composition of any previous embodiment, wherein the anti-viral agent is specific to, or derived from, a virus is selected from:
b) a double stranded (ds) DNA viruses (e.g. Adenoviruses, Herpesviruses, Poxviruses);
c) a single stranded (ss) DNA viruses (+ strand or “sense”) DNA (e.g. Parvoviruses);
d) a dsRNA viruses (e.g. Reoviruses);
e) a (+)ssRNA viruses (+ strand or sense) RNA (e.g. Picornaviruses, Togaviruses);
f) a (−)ssRNA viruses (− strand or antisense) RNA (e.g. Orthomyxoviruses, Rhabdoviruses);
g) a ssRNA-RT viruses (+ strand or sense) RNA with DNA intermediate in life-cycle (e.g. Retroviruses); or
h) a dsDNA-RT viruses DNA with RNA intermediate in life-cycle (e.g. Hepadnaviruses)

Embodiment 14. The composition of any previous embodiment, wherein the anti-viral agent is derived from a respiratory virus, a skin virus, a foodborne virus, a sexually transmitted virus, or an oncolytic virus, or a combination thereof

Embodiment 15. The composition of embodiment 14, wherein the respiratory virus is selected from Rhinovirus, influenza virus, respiratory syncytial virus, and coronavirus.

Embodiment 16. The composition of embodiment 14, wherein the skin virus is selected from molluscum contagiosum, herpes simplex vius-1, and varicella-zoster virus.

Embodiment 17. The composition of embodiment 14, wherein the foodborne virus is selected from hepatitis A, norovirus, and rotavirus.

Embodiment 18. The composition of embodiment 14, wherein the sexually transmitted virus is selected from human papillomavirus, hepatitis B, genital herpes, and human immunodeficiency virus.

Embodiment 19. The composition of embodiment 14, wherein the oncolytic virus is human papilloma virus or hepatitis B.

Embodiment 20. The composition of embodiment 12, wherein the linker comprises glycosyl-phosphatidylinositol (GPI) or a B7-1 antigen (B7-1) cytoplasmic tail.

Embodiment 21. The composition of embodiment 3, wherein the viral antigen is a transmembrane peptide expressed in the cell.

Embodiment 22. The composition of embodiments 3-21, wherein the viral antigen is immunogenic to a human.

Embodiment 23. The composition of embodiment 3-22, wherein the viral antigen is a peptide derived from a coronavirus.

Embodiment 24. The composition of embodiment 23, wherein the coronavirus is severe acute respiratory syndrome coronavirus 2 (SARS-CoV-2), or a variant thereof.

Embodiment 25. The composition of embodiment 23 or 24, wherein the peptide is selected from a spike protein, a membrane protein, or a nucleoprotein derived from the coronavirus.

Embodiment 26. The composition of embodiments 25, wherein the cell comprises mRNA encoding the peptide.

Embodiment 27. The composition of embodiment 26, wherein the mRNA comprises an mRNA sequence that is at least 80% identical to SEQ ID NO: 1.

Embodiment 28. The composition of embodiment 26, wherein the mRNA comprises an mRNA sequence that is at least 85% identical to SEQ ID NO: 1.

Embodiment 29. The composition of embodiment 26, wherein the mRNA comprises an mRNA sequence that is at least 90% identical to SEQ ID NO: 1.

Embodiment 30. The composition of embodiment 26, wherein the mRNA comprises an mRNA sequence that is at least 91%, 92%, 93%, 94%, 95%, 96%, 97%, 98%, or 99% identical to SEQ ID NO: 1.

Embodiment 31. The composition of embodiment 26, wherein the mRNA comprises an mRNA sequence that is at least 100% identical to SEQ ID NO: 1.

Embodiment 32. The composition of embodiment 23-26, wherein the peptide comprises an amino acid sequence that is at least 70%, 75%, 80%, 85%, 90%, 91%, 92%, 93%, 94%, 95%, 96%, 97%, 98%, 99%, or 100% identical to SEQ ID NO: 2 or 8.

Embodiment 33. The composition of embodiments 26-32, wherein the mRNA has a half-life of 3-5 days.

Embodiment 34. The composition of embodiments 26-32, wherein the mRNA encodes a fusion protein comprising an albumin peptide.

Embodiment 35. The composition of embodiments 26-32, wherein the mRNA encodes a fusion protein comprising an immune-modulator.

Embodiment 36. The composition of embodiment 35, wherein the immune-modulator is an activator of an immune response in a subject.

Embodiment 37. The composition of embodiment 36, wherein the immune-modulator is granulocyte-macrophage colony-stimulating factor (GM-CSF) or a cytokine, or a combination thereof.

Embodiment 38. The composition of any previous embodiment, wherein the cell further comprises one or more homing receptors.

Embodiment 39. The composition of embodiment 38, wherein the one or more homing receptors is tethered to a surface of the cell by a linker selected from a chemical linker, a peptide linker, or a polymer.

Embodiment 40. The composition of embodiment 39, wherein the linker comprises glycosyl-phosphatidylinositol (GPI) or a B7-1 antigen (B7-1) cytoplasmic tail.

Embodiment 41. The composition of embodiment 38, wherein the one or more homing receptors is expressed on a surface of the cell.

Embodiment 42. The composition of embodiment 41, wherein the one or more homing receptors is genetically modified to increase expression of the one or more homing receptors on a surface of the cell.

Embodiment 43. The composition of embodiments 38-42, wherein the one or more homing receptors is specific to one or more ligands expressed on one or more cells in lymph tissue.

Embodiment 44. The composition of embodiment 43, wherein the one or more cells in the lymph tissue comprises endothelial cells, lymphocytes, macrophages, or reticular cells, or a combination thereof

Embodiment 45. The composition of embodiments 38-44, wherein the one or more homing receptors comprise two or more homing receptors specific to two or more ligands that are not the same.

Embodiment 46. The composition of embodiments 38-45, wherein the one or more homing receptors is selected from C-X-C chemokine receptor type 3 (CXCR3), leukosialin (CD43), CD44 antigen (CD44), C-C chemokine receptor type 7 (CCR7), L-selectin (CD62L), lymphocyte function-associated antigen 1 (LFA-1), or very late antigen-4 (VLA4).

Embodiment 47. The composition of embodiments 38-46, wherein the one or more homing receptors comprise L-Selectin (CD62L) and C-C chemokine receptor type 7 (CCR7).

Embodiment 48. The composition of embodiments 38-46, wherein the one or more homing receptors is specific to a ligand expressed in endothelial cells of the lymph tissue, and the viral antigen is effective to activate an immune response in a subject against a coronavirus, when composition is administered to the subject.

Embodiment 49. The composition of any previous embodiment, wherein the cell further comprises one or more immune-modulators.

Embodiment 50. The composition of embodiment 49, wherein the one or more immune-modulators is tethered to a surface of the cell.

Embodiment 51. The composition of embodiment 50, wherein the one or more immune-modulators is tethered to a surface of the cell using a linker comprising glycosyl-phosphatidylinositol (GPI) or a B7-1 antigen (B7-1) cytoplasmic tail.

Embodiment 52. The composition of embodiments 49-51, wherein the one or more immune-modulators is expressed on a surface of the cell.

Embodiment 53. The composition of embodiments 49-52, wherein the one or more immune-modulators is selected from the group consisting of granulocyte-macrophage colony-stimulating factor (GM-CSF), tumor necrosis factor alpha (TNF-alpha), lymphotoxin alpha (LTA), lymphotoxin beta (LTB), TNF superfamily member 4 (TNFSF4), CD40 ligand (CD40LG), fas ligand (FASLG), CD70 molecule (CD70), TNF superfamily member 8 (TNFSF8), TNF superfamily member 9 (TNFSF9), TNF superfamily member 10 (TNFSF10), TNF superfamily member 11 (TNFSF11), TNF superfamily member 12 (TNFSF12), TNF superfamily member 13 (TNFSF13), TNF superfamily member 13b (TNFSF13B), TNF superfamily member 14 (TNFSF14), TNF superfamily member 15 (TNFSF15), TNF superfamily 18 (TNFSF18), ectodysplasin A (EDA), cytokines, and viral antigen proteins.

Embodiment 54. The composition of embodiment 49-53, wherein the one or more immune-modulators is a fusion protein comprising an albumin peptide.

Embodiment 55. The composition of embodiments 1-54, wherein the composition is isolated.

Embodiment 56. The composition of embodiments 1-54, wherein the composition is purified.

Embodiment 57. The composition of embodiments 1-54, comprising a plurality of the cells in a suspension or in a cell culture, or both.

Embodiment 58. The composition of embodiments 1-57, wherein the composition is cryopreserved or was previously cryopreserved for at least 48 hours.

Embodiment 59. A method of delivering the composition of any previous embodiment, the method comprising administering to a subject in need thereof the composition by systemic delivery or direct delivery.

Embodiment 60. The method of embodiment 59, wherein systemic delivery comprises intravenous delivery or inhalation, and wherein direct delivery comprises intramuscular, intraperitoneal, and intra-lymph node, delivery.

Embodiment 61. The method of embodiments 59-60, further comprising substantially immunizing the subject from an infection by a live virus comprising the composition subsequent to the delivery.

Embodiment 62. A method of preventing viral infection in a subject, the method comprising administering the composition of embodiments 1-58 to the subject, thereby substantially immunizing the subject from an infection by a live virus comprising the composition.

Embodiment 63. A method of treating an acute viral infection in a subject, the method comprising administering the composition of embodiments 1-58 to the subject, thereby reducing the viral load in the subject.

Embodiment 64. A method of preventing a disease caused by a coronavirus in a subject, the method comprising administering the composition of embodiments 1-58 to the subject, thereby preventing the disease caused by the coronavirus.

Embodiment 65. A method of treating a disease caused by a coronavirus in a subject, the method comprising administering the composition of embodiments 1-58 to the subject, thereby treating the disease caused by the coronavirus.

Embodiment 66. The method of embodiments 64 and 65, wherein the disease is coronavirus disease of 2019 (COVID-19).

Embodiment 67. The method of embodiments 59-66, further comprising: (a) receiving the composition stored in a suspension at 4 degrees Celsius for at least 48 hours, wherein the composition has a slowed or stopped biological activity; and (b) removing the composition from the suspension, thereby reviving the biological activity of the composition.

### Methods of Manufacturing

Disclosed herein are methods of utilizing a cytoplast to produce compositions in accordance with the embodiments below:

Embodiment 1. A method of manufacturing a composition, the method comprising:

(a) introducing a first nucleic acid encoding a first viral antigen or an anti-viral antibody to a parent cell, the parent cell comprising:
i) a nucleus; and
ii) one or more intracellular organelles for protein synthesis or protein secretion; and

(b) mechanically removing the nucleus from the parent stem cell to produce an enucleated stem cell, wherein the enucleated stem cell comprises the one or more intracellular organelles.

Embodiment 2. A method of manufacturing a composition, the method comprising:
(a) introducing a first nucleic acid encoding a first viral antigen or an anti-viral antibody to an enucleated stem cell, the enucleated stem cell comprising one or more intracellular organelles for protein synthesis or protein secretion of the first viral antigen or the anti-viral antibody; and
(b) expressing the first viral antigen or the anti-viral antibody in the enucleated stem cell.

Embodiment 3. The method of embodiments 1 and 2, wherein the first viral antigen is expressed at a surface of the enucleated stem cell.

Embodiment 4. The method of any previous embodiments, wherein the first viral antigen or the anti-viral antibody is secretory.

Embodiment 5. The method of any previous embodiments, further comprising storing the enucleated stem cell in a suspension at a temperature below the freezing temperature of the suspension for at least 24 hours, 48 hours, or 96 hours.

Embodiment 6. The method of any previous embodiments, further comprising introducing a second nucleic acid encoding a second viral antigen, wherein the first and second nucleic acids are not identical and the first and second viral antigens are not identical.

Embodiment 7. The method of any previous embodiments, further comprising introducing a plurality of nucleic acids encoding a plurality of viral antigens that differ from the first viral antigen.

Embodiment 8. The method of any previous embodiments, wherein the nucleic acid is a messenger RNA (mRNA).

Embodiment 9. The method of any previous embodiments, wherein the nucleic acid is DNA.

Embodiment 10. The method of any previous embodiments, wherein the first viral antigen is derived from a mammal.

Embodiment 11. The method of any previous embodiment, wherein the antiviral antibody is specific to a coronavirus.

Embodiment 12. The method of any previous embodiment, wherein the first viral antigen is an attenuated viral particle derived from a coronavirus.

Embodiment 13. The method of any previous embodiment, wherein the first viral antigen is tethered to a surface of the enucleated stem cell by a linker selected from a chemical linker, a peptide linker, or a polymer.

Embodiment 14. The method of embodiment 13, wherein the linker comprises glycosyl-phosphatidylinositol (GPI) or a B7-1 antigen (B7-1) cytoplasmic tail.

Embodiment 15. The method of any previous embodiment, wherein the first viral antigen is a transmembrane peptide expressed in the enucleated stem cell.

Embodiment 16. The method of any previous embodiment, wherein the first viral antigen is immunogenic to a human.

Embodiment 17. The method of any previous embodiment, wherein the first viral antigen is a peptide derived from a coronavirus.

Embodiment 18. The method of embodiment 17, wherein the peptide is selected from a spike protein, a membrane protein, or a nucleoprotein derived from the coronavirus.

Embodiment 19. The method of embodiment 18, wherein the coronavirus is severe acute respiratory syndrome coronavirus 2 (SARS-CoV-2), or a variant thereof

Embodiment 20. The method of embodiments 17-19, wherein the enucleated stem cell comprises mRNA encoding the peptide.

Embodiment 21. The method of embodiment 20, wherein the mRNA comprises an mRNA sequence that is at least 80% identical to SEQ ID NO: 1.

Embodiment 22. The method of embodiment 20, wherein the mRNA comprises an mRNA sequence that is at least 85% identical to SEQ ID NO: 1.

Embodiment 23. The method of embodiment 20, wherein the mRNA comprises an mRNA sequence that is at least 90% identical to SEQ ID NO: 1.

Embodiment 24. The method of embodiment 20, wherein the mRNA comprises an mRNA sequence that is at least 91%, 92%, 93%, 94%, 95%, 96%, 97%, 98%, or 99% identical to SEQ ID NO: 1.

Embodiment 25. The method of embodiment 20, wherein the mRNA comprises an mRNA sequence that is at least 100% identical to SEQ ID NO: 1.

Embodiment 26. The method of embodiments 17-20, wherein the peptide comprises an amino acid sequence that is at least 70%, 75%, 80%, 85%, 90%, 91%, 92%, 93%, 94%, 95%, 96%, 97%, 98%, 99%, or 100% identical to SEQ ID NO: 2.

Embodiment 27. The method of embodiments 20-26, wherein the mRNA has a half-life of 3-5 days.

Embodiment 28. The method of embodiments 20-26, wherein the mRNA encodes a fusion protein comprising an albumin peptide.

Embodiment 29. The method of embodiments 20-26, wherein the mRNA encodes a fusion protein comprising an immune-modulator.

Embodiment 30. The method of embodiment 29, wherein the immune-modulator is an activator of an immune response in a subject.

Embodiment 31. The method of embodiment 30, wherein the immune-modulator is granulocyte-macrophage colony-stimulating factor (GM-CSF) or a cytokine, or a combination thereof.

Embodiment 32. The method of any previous embodiment, wherein the enucleated stem cell further comprises one or more homing receptors.

Embodiment 33. The method of embodiment 32, wherein the one or more homing receptors is tethered to a surface of the enucleated stem cell by a linker selected from a chemical linker, a peptide linker, or a polymer.

Embodiment 34. The method of embodiment 33, wherein the linker comprises glycosyl-phosphatidylinositol (GPI) or a B7-1 antigen (B7-1) cytoplasmic tail.

Embodiment 35. The method of embodiment 32, wherein the one or more homing receptors is expressed on a surface of the enucleated stem cell.

Embodiment 36. The method of embodiment 32-35, wherein the one or more homing receptors is genetically modified to increase expression of the one or more homing receptors on a surface of the enucleated stem cell.

Embodiment 37. The method of embodiments 32-36, wherein the one or more homing receptors is specific to one or more ligands expressed on one or more cells in lymph tissue.

Embodiment 38. The method of embodiment 37, wherein the one or more cells in the lymph tissue is selected from endothelial cells, lymphocytes, macrophages, or reticular cells, or a combination thereof

Embodiment 39. The method of embodiments 32-38, wherein the one or more homing receptors comprise two or more homing receptors specific to two or more ligands that are not the same.

Embodiment 40. The method of embodiments 32-39, wherein the one or more homing receptors is selected from C-X-C chemokine receptor type 3 (CXCR3), leukosialin (CD43), CD44 antigen (CD44), C-C chemokine receptor type 7 (CCR7), L-selectin (CD62L), lymphocyte function-associated antigen 1 (LFA-1), or very late antigen-4 (VLA4).

Embodiment 41. The method of embodiments 32-40, wherein the one or more homing receptors comprise L-Selectin (CD62L) and C-C chemokine receptor type 7 (CCR7).

Embodiment 42. The method of embodiment 32-41, wherein the one or more homing receptors is specific to a ligand expressed in endothelial cells of the lymph tissue, and the viral antigen is effective to activate an immune response in a subject against a coronavirus, when the viral antigen is administered to the subject.

Embodiment 43. The method of any previous embodiment, wherein the enucleated stem cell further comprises one or more immune-modulators.

Embodiment 44. The method of embodiment 43, wherein the one or more immune-modulators is tethered to a surface of the enucleated stem cell.

Embodiment 45. The method of embodiment 44, wherein the one or more immune-modulators is tethered to a surface of the enucleated stem cell using a linker comprising glycosyl-phosphatidylinositol (GPI) or a B7-1 antigen (B7-1) cytoplasmic tail.

Embodiment 46. The method of embodiments 43-45, wherein the one or more immune-modulators is expressed on a surface of the enucleated stem cell.

Embodiment 47. The method of embodiment 43-46, wherein the one or more immune-modulators is selected from the group consisting of granulocyte-macrophage colony-stimulating factor (GM-CSF), tumor necrosis factor alpha (TNF-alpha), lymphotoxin alpha (LTA), lymphotoxin beta (LTB), TNF superfamily member 4 (TNFSF4), CD40 ligand (CD40LG), fas ligand (FASLG), CD70 molecule (CD70), TNF superfamily member 8 (TNFSF8), TNF superfamily member 9 (TNFSF9), TNF superfamily member 10 (TNFSF10), TNF superfamily member 11 (TNFSF11), TNF superfamily member 12 (TNFSF12), TNF superfamily member 13 (TNFSF13), TNF superfamily member 13b (TNFSF13B), TNF superfamily member 14 (TNFSF14), TNF superfamily member 15 (TNFSF15), TNF superfamily 18 (TNFSF18), ectodysplasin A (EDA), cytokines, and viral antigen proteins.

Embodiment 48. The method of embodiments 43-47, wherein the one or more immune-modulators is a fusion protein comprising an albumin peptide.

Embodiment 49. The method of embodiments 1-48, wherein the method further comprises isolating the enucleated stem cell.

Embodiment 50. The method of embodiments 1-48, wherein the method further comprises purifying the enucleated stem cell.

Embodiment 51. The method of embodiments 1-48, wherein the enucleated stem cell is a plurality of enucleated stem cells in a suspension or in a cell culture, or both.

Embodiment 52. The method of embodiments 1-48, wherein the method further comprises cryopreserving the enucleated stem cell for at least 48 hours.

Embodiment 53. A method of preventing a disease caused by a coronavirus in a subject, the method comprising administering the composition of embodiments 1-48 to the subject, thereby preventing the disease caused by the coronavirus.

Embodiment 54. A method of treating a disease caused by a coronavirus in a subject, the method comprising administering the composition of embodiments 1-48 to the subject, thereby treating the disease caused by the coronavirus.

Embodiment 55. The method of embodiments 53 and 54, wherein the disease is coronavirus disease of 2019 (COVID-19).

Embodiment 56. The method of embodiments 1-55, further comprising: (a) receiving the enucleated stem cell stored in a suspension at 4 degrees Celsius for at least 48 hours, wherein the enucleated stem cell has a slowed or stopped biological activity; and (b) removing the enucleated stem cell from the suspension, thereby reviving the biological activity of the enucleated stem cell.

### Methods of Virus Trapping

Embodiment 1. A method of clearing a pathogen in a subject, the method comprising:

(a) administering to a subject in need thereof a plurality of cells substantially free of nuclei;

(b) sequestering a pathogen in a tissue of the subject by:
i. permitting infection in vivo of the plurality of cells administered to the subject in (a) by the pathogen; and
ii. once the plurality of cells are infected, preventing propagation of the pathogen;
iii. removing the plurality of cells from the subject by phagocytosis, thereby eliminating the pathogen from the subject.

Embodiment 2. The method of embodiment 1, wherein a number of pathogens is reduced in a dose-dependent manner to administration in (a) of the plurality of cells.

Embodiment 3. The method of any previous embodiment, wherein the plurality of cells expresses one or more immune-modulators, and wherein the one or more immune-modulators is expressed at a surface of a cell in the plurality of cells, or secreted by a cell in the plurality of cells.

Embodiment 4. The method of embodiment 3, wherein the one or more immune-modulators is tethered to a surface of a cell in the plurality of cells.

Embodiment 5. The method of embodiment 4, wherein the one or more immune-modulators is tethered to a surface of a cell using a linker comprising glycosyl-phosphatidylinositol (GPI) or a B7-1 antigen (B7-1) cytoplasmic tail.

Embodiment 6. wherein the one or more immune-modulators is selected from the group consisting of granulocyte-macrophage colony-stimulating factor (GM-CSF), tumor necrosis factor alpha (TNF-alpha), lymphotoxin alpha (LTA), lymphotoxin beta (LTB), TNF superfamily member 4 (TNFSF4), CD40 ligand (CD40LG), fas ligand (FASLG), CD70 molecule (CD70), TNF superfamily member 8 (TNFSF8), TNF superfamily member 9 (TNFSF9), TNF superfamily member 10 (TNFSF10), TNF superfamily member 11 (TNFSF11), TNF superfamily member 12 (TNFSF12), TNF superfamily member 13 (TNFSF13), TNF superfamily member 13b (TNFSF13B), TNF superfamily member 14 (TNFSF14), TNF superfamily member 15 (TNFSF15), TNF superfamily 18 (TNFSF18), ectodysplasin A (EDA), one or more cytokines, and viral antigen proteins.

Embodiment 7. The method of embodiment 6, wherein the one or more cytokines is selected from interleukin 10 and interleukin 12.

Embodiment 8. The method of any previous embodiment, wherein the plurality of cells are engineered to express one or more homing receptors specific to a target tissue, and wherein the one or more homing receptors is expressed at a surface of a cell in the plurality of cells, or secreted by a cell in the plurality of cells.

Embodiment 9. The method of embodiment 8, wherein the target tissue is the lung or lymph tissue.

Embodiment 10. The method of embodiment 9, wherein the one or more homing receptors targets endothelial cells, lymphocytes, macrophages, or reticular cells, or a combination thereof, in the lymph tissue.

Embodiment 11. The method of embodiments 8-10, wherein the one or more homing receptors is tethered to a surface of a cell in the plurality of cells by a linker selected from a chemical linker, a peptide linker, or a polymer.

Embodiment 12. The method of embodiment 11, wherein the linker comprises glycosyl-phosphatidylinositol (GPI) or a B7-1 antigen (B7-1) cytoplasmic tail.

Embodiment 13. The method of embodiments 8-12, wherein the one or more homing receptors is genetically modified to increase expression of the one or more homing receptors on a surface of a cell in the plurality of cells.

Embodiment 14. The method of embodiments 8-13, wherein the one or more homing receptors comprise two or more homing receptors specific to two or more target tissues that are not the same.

Embodiment 15. The method of embodiments 8-14, wherein the one or more homing receptors is selected from C-X-C chemokine receptor type 3 (CXCR3), leukosialin (CD43), CD44 antigen (CD44), C-C chemokine receptor type 7 (CCR7), L-selectin (CD62L), lymphocyte function-associated antigen 1 (LFA-1), or very late antigen-4 (VLA4).

Embodiment 16. The method of embodiment 1, wherein a cell of the plurality of cells comprises a viral antigen.

Embodiment 17. The method of embodiment 16, wherein the viral antigen is expressed on a surface of the cell in the plurality of cells.

Embodiment 18. The method of embodiment 16, wherein the viral antigen is tethered to a surface of a cell in the plurality of cells by a linker selected from a chemical linker, a peptide linker, or a polymer.

Embodiment 19. The method of embodiment 18, wherein the linker comprises glycosyl-phosphatidylinositol (GPI) or a B7-1 antigen (B7-1) cytoplasmic tail.

Embodiment 20. The method of embodiments 16-19, wherein the viral antigen is a transmembrane peptide expressed in a cell in the plurality of cells.

Embodiment 21. The method of embodiments 16-20, wherein the viral antigen is immunogenic to a human.

Embodiment 22. The method of embodiments 16-21, wherein the viral antigen is a peptide derived from a coronavirus.

Embodiment 23. The method of embodiment 22, wherein the peptide is selected from a spike protein, a membrane protein, or a nucleoprotein derived from the coronavirus.

Embodiment 24. The method of embodiment 23, wherein the coronavirus is severe acute respiratory syndrome coronavirus 2 (SARS-CoV-2), or a variant thereof

Embodiment 25. The method of embodiments 22-24, wherein a cell in the plurality of cells comprises mRNA encoding the peptide.

Embodiment 26. The method of embodiment 25, wherein the mRNA comprises an mRNA sequence that is at least 80% identical to SEQ ID NO: 1.

Embodiment 27. The method of embodiment 25, wherein the mRNA comprises an mRNA sequence that is at least that is at least 85% identical to SEQ ID NO: 1.

Embodiment 28. The method of embodiment 25, wherein the mRNA comprises an mRNA sequence that is at least 90% identical to SEQ ID NO: 1.

Embodiment 29. The method of embodiment 25, wherein the mRNA comprises an mRNA sequence that is at least 91%, 92%, 93%, 94%, 95%, 96%, 97%, 98%, or 99% identical to SEQ ID NO: 1.

Embodiment 30. The method of embodiment 25, wherein the mRNA comprises an mRNA sequence that is at least 100% identical to SEQ ID NO: 1.

Embodiment 31. The method of embodiment 22-25, wherein the peptide comprises an amino acid sequence that is at least 70%, 75%, 80%, 85%, 90%, 91%, 92%, 93%, 94%, 95%, 96%, 97%, 98%, 99%, or 100% identical to SEQ ID NO: 2.

Embodiment 32. The method of embodiments 25-31, wherein the mRNA has a half-life of 3-5 days.

Embodiment 33. The method of embodiments 25-31, wherein the mRNA encodes a fusion protein comprising an albumin peptide.

Embodiment 34. The method of embodiments 25-31, wherein the mRNA encodes a fusion protein comprising an immune-modulator.

Embodiment 35. The method of embodiment 34, wherein the immune-modulator is an activator of an immune response in a subject.

Embodiment 36. The method of embodiment 34, wherein the immune-modulator is granulocyte-macrophage colony-stimulating factor (GM-CSF) or a cytokine, or a combination thereof.

Embodiment 37. The method of any previous embodiment, wherein the pathogen is a live virus selected from a respiratory virus, a skin virus, a foodborne virus, a sexually transmitted virus, or an oncolytic virus, or a combination thereof

Embodiment 38. The method of embodiment 37, wherein the respiratory virus is selected from Rhinovirus, influenza virus, respiratory syncytial virus, and coronavirus.

Embodiment 39. The method of embodiment 38, wherein the coronavirus is severe acute respiratory syndrome coronavirus 2 (SARS-CoV-2), or a variant thereof

Embodiment 40. The method of embodiment 37, wherein the skin virus is selected from molluscum contagiosum, herpes simplex vius-1, and varicella-zoster virus.

Embodiment 41. The method of embodiment 37, wherein the foodborne virus is selected from hepatitis A, norovirus, and rotavirus.

Embodiment 42. The method of embodiment 37, wherein the sexually transmitted virus is selected from human papillomavirus, hepatitis B, genital herpes, and human immunodeficiency virus.

Embodiment 43. The method of embodiment 37, wherein the oncolytic virus is human papilloma virus or hepatitis B.

Embodiment 44. The method of any previous embodiment, wherein administering in (a) is intra-peritoneal, intra-tumoral, intra-venous, intra-lymphatic, intra-muscular, or inhalation.

Embodiment 45. The method of embodiment 1, wherein the pathogen is a live virus is selected from:
a) a double stranded (ds) DNA viruses (e.g. Adenoviruses, Herpesviruses, Poxviruses);
b) a single stranded (ss) DNA viruses (+ strand or “sense”) DNA (e.g. Parvoviruses);
c) a dsRNA viruses (e.g. Reoviruses);
d) a (+)ssRNA viruses (+ strand or sense) RNA (e.g. Picornaviruses, Togaviruses);
e) a (−)ssRNA viruses (− strand or antisense) RNA (e.g. Orthomyxoviruses, Rhabdoviruses);
f) a ssRNA-RT viruses (+ strand or sense) RNA with DNA intermediate in life-cycle (e.g. Retroviruses); or
g) a dsDNA-RT viruses DNA with RNA intermediate in life-cycle (e.g. Hepadnaviruses)

Embodiment 46. The method of embodiments 1-36, wherein the pathogen is a bacteria, virus, parasite, fugus, autoantibody, antibody, poisonous substance, toxic substance, or a combination thereof

Embodiment 47. The method of embodiments 1-46, further comprising: (a) receiving the plurality of cells stored in a suspension at 4 degrees Celsius for at least 48 hours, wherein the plurality of cells has a slowed or stopped biological activity; and (b) removing the plurality of cells from the suspension, thereby reviving the biological activity of the plurality of cells.

### Compositions for Pathogen Trapping

Embodiment 1. A cell without a nucleus, the cell comprising: one or more intracellular organelles for synthesis of a receptor for a pathogenic antigen or a pathogen antigen-binding fragment thereof in absence of the nucleus.

Embodiment 2. The cell without the nucleus of embodiment 1, wherein the one or more intracellular organelles is an endoplasmic reticulum or a Golgi apparatus.

Embodiment 3. The cell without the nucleus of any one of embodiments 1-2, wherein the receptor for the pathogenic antigen or the pathogen antigen-binding fragment thereof is coupled to a surface of the cell without the nucleus.

Embodiment 4. The cell without the nucleus of any one of embodiments 1-3, wherein the receptor for the pathogenic antigen or the pathogen antigen-binding fragment thereof comprises a transmembrane domain that couples the receptor for the pathogenic antigen or the pathogen antigen-binding fragment thereof to the surface of the cell without the nucleus.

Embodiment 5. The cell without the nucleus of any one of embodiments 1-4, wherein the cell without the nucleus further comprises an immune-modulator comprising granulocyte-macrophage colony-stimulating factor.

Embodiment 6.The cell without the nucleus of any one of embodiments 1-5, wherein the cell without the nucleus has a diameter that is between about 1 micrometers (μm) to 100 μm.

Embodiment 7. The cell without the nucleus of embodiment 6, wherein the diameter is about 8 μm.

Embodiment 8.The cell without the nucleus of any one of embodiments 1-7, wherein the cell without the nucleus is viable following cryohibernation for at least 24 hours.

Embodiment 9. The cell without the nucleus of any one of embodiments 1-7, wherein the cell without the nucleus is viable following cryopreservation for at least 24 hours

Embodiment 10. The cell without the nucleus of any one of embodiments 1-9, wherein the cell without the nucleus is cryopreserved, cryohybernated, or lyophilized.

Embodiment 11. The cell without the nucleus of any one of embodiments 1-10, wherein the cell without a nucleus is isolated or purified.

Embodiment 12. The cell without the nucleus of any one of embodiments 1-11, wherein the pathogenic antigen is an antigen of a coronavirus.

Embodiment 13. The cell without the nucleus of embodiment 12, wherein the coronavirus is SARS-CoV-2.

Embodiment 14. The cell without the nucleus of any one of embodiments 1-13, further comprising a neutralizing antibody that blocks binding between the pathogen antigen and its natural receptor produced by a host cell.

Embodiment 15. The cell without the nucleus of any one of embodiments 1-14, further comprising one or more immune-modulators.

Embodiment 16. The cell without the nucleus of embodiment 15, wherein the one or more immune-modulators is tethered to a surface of a cell without the nucleus using a linker comprising glycosyl-phosphatidylinositol (GPI) or a B7-1 antigen (B7-1) cytoplasmic tail.

Embodiment 17. The cell without the nucleus of embodiment 15, wherein the one or more immune-modulators is selected from the group consisting of granulocyte-macrophage colony-stimulating factor (GM-CSF), tumor necrosis factor alpha (TNF-alpha), lymphotoxin alpha (LTA), lymphotoxin beta (LTB), TNF superfamily member 4 (TNFSF4), CD40 ligand (CD40LG), fas ligand (FASLG), CD70 molecule (CD70), TNF superfamily member 8 (TNFSF8), TNF superfamily member 9 (TNFSF9), TNF superfamily member 10 (TNFSF10), TNF superfamily member 11 (TNFSF11), TNF superfamily member 12 (TNFSF12), TNF superfamily member 13 (TNFSF13), TNF superfamily member 13b (TNFSF13B), TNF superfamily member 14 (TNFSF14), TNF superfamily member 15 (TNFSF15), TNF superfamily 18 (TNFSF18), ectodysplasin A (EDA), one or more cytokines, and viral antigen proteins.

Embodiment 18. The cell without the nucleus of any one of embodiments 1-17, further comprising one or more homing receptors specific to a target tissue.

Embodiment 19. The cell without the nucleus of embodiment 18, wherein the one or more homing receptors targets endothelial cells, lymphocytes, macrophages, or reticular cells, or a combination thereof, in the lymph tissue.

Embodiment 20. The cell without the nucleus of embodiment 18, wherein the one or more homing receptors is tethered to a surface of a cell in the plurality of cells by a linker selected from a chemical linker, a peptide linker, or a polymer.

Embodiment 21. The cell without the nucleus of embodiment 20, wherein the linker comprises glycosyl-phosphatidylinositol (GPI) or a B7-1 antigen (B7-1) cytoplasmic tail.

Embodiment 22. The cell without the nucleus of any one of embodiments 18-21, wherein the one or more homing receptors is selected from C-X-C chemokine receptor type 3 (CXCR3), leukosialin (CD43), CD44 antigen (CD44), C-C chemokine receptor type 7 (CCR7), L-selectin (CD62L), lymphocyte function-associated antigen 1 (LFA-1), or very late antigen-4 (VLA4).

Embodiment 23. The cell without the nucleus of any one of embodiments 1-23, further comprising a viral antigen.

Embodiment 24. A pharmaceutical formulation comprising:

the cell without the nucleus of any one of embodiments 1-23 or a plurality of the cell without the nucleus of any one of embodiments 1-23; and

a pharmaceutically acceptable: excipient, diluent, or carrier.

Embodiment 25. A method of reducing an infection by a pathogen in a subject, the method comprising: administering to a subject the cell without the nucleus of any one of embodiments 1-23 or the pharmaceutical formulation of embodiment 24, thereby trapping a pathogen having the pathogen antigen in the cell and preventing the pathogen from propagating within the cell.

Embodiment 26. The method of embodiment 25, wherein the pathogen is cleared from the subject in 14 days or fewer following administration.

Embodiment 27. The method of any one of embodiments 26-27, wherein the cell without the nucleus releases the neutralizing antibody, thereby blocking binding between the pathogen antigen of the pathogen and its natural receptor produced by a host cell.

Embodiment 28. The method of any one of embodiments 26-28, wherein the cell without the nucleus presents the viral antigen, thereby immunizing the subject from an infection by the pathogen.

### VII. EXAMPLES

The following examples are included for illustrative purposes only and are not intended to limit the scope of the invention.

### Example 1

### Method of Producing an Anti-Viral Composition for Coronavirus

Lentiviral-mediated transfection of stem cell (e.g., mesenchymal stem cell) with a heterologous nucleic acid encoding an attenuated coronavirus antigen is performed. Next, enucleation of the stem cell by methods described in Example 7 is performed. Enucleated stem cells expressing the attenuated coronavirus antigen at the surface of the cell are verified using flow cytometry. Successfully enucleated stem cells expressing the attenuated corona virus antigen (referred to as “cytoplast” in this example) are isolated and purified according to known methods. Optionally, the cytoplasts are cryopreserved using the methods provided in Example 4. The cytoplasts described above are useful as a vaccine for the preventing of coronavirus infection.

A second anti-viral composition for coronavirus is produced using similar methodology as above, but instead of an attenuated coronavirus antigen, an antibody against coronavirus is expressed in the stem cell. Alternatively, or in addition, a small molecule against coronavirus is loaded into the enucleated stem cell using electroporation (or comparable methods known in the art). The successfully enucleated stem cells expressing the anti-viral antibody against coronavirus and/or the small molecule against coronavirus (referred to as “cytoplast” in this example) are isolated and purified according to known methods. Optionally, the cytoplasts are cryopreserved using the methods provided in Example 4. The cytoplasts described above are useful to treat acute coronavirus infection.

### Example 2

### Preventing Coronavirus Infection in a Subject

The anti-viral composition described in Example 1 expressing the attenuated coronavirus or a peptide fragment of a coronaviral protein is formulated for intravenous administration. The attenuated coronavirus or the peptide fragment of the coronaviral protein may be encoded from mRNA encapsulated in the cytoplasts described herein. In some embodiments, the anti-viral composition is formulated for intramuscular administration. In some embodiments, the subject receives a first and a second dose of the anti-viral compositions. In some embodiments, the second dose of the anti-viral composition is administered at least 1 day, 2 day, 3 day, 4 day, 1 week, 2 weeks, 3 weeks, 4 weeks, 1 month, 2 months, 3 months, or 4 months after the administration of the first does. The formulation is administered intravenously to a subject. For example, administration to a human subject would be performed at least 5 times when the subject is a child. In some embodiments, the formulation is administered to the subject when the subject is age 2 months old, 4 months old, 6 months old, between 15-18 months old, and between 4-6 years old. In this example, the subject becomes immunized from a coronavirus infection.

### Example 3

### Treating an Acute Coronavirus Infection in a Subject

The anti-viral composition described in Example 1 expressing the anti-coronavirus antibody (e.g., neutralizing antibody) or small molecule against coronavirus is formulated for intravenous administration. The formulation is administered intravenously to a subject infected, or suspected of being infected with, coronavirus. In some embodiments, administration is performed more than once. For example administration may be performed every day, every two days, every week, every two weeks, every month, every two months, for a period of time (for e.g., 1 year). In this example, the coronavirus infection is reduced in the subject.

Alternatively, or in addition, enucleated stem cells (e.g., mesenchymal stem cell) without a payload are formulated for intravenous administration. The formulation is administered intravenously to a subject infected, or suspected of being infected with, coronavirus. In some embodiments, administration is performed more than one. For example administration may be performed every day, every two days, every week, every two weeks, every month, every two months, for a period of time (for e.g., 1 year). In this example, the cytoplasts are infected with the coronavirus in vivo and become trapped in the cytoplast. Cytoplasts lacking a nucleus lack the genetic material required for coronavirus replication and propagation, thereby preventing the coronavirus from further infection. In this example the coronavirus infection is reduced.

### Example 4

### Producing Cytoplasts from Mammalian Cells

Cytoplasts can be generated from allogenic or autologous donor-derived cells, and can be used for disease treatment as well as for diagnostics. As a proof of concept, the enucleation efficiency and recovery rate of various types of mammalian cells (e.g., mesenchymal stem cells, neutrophils, fibroblast, and natural killer cells) was determined. After removal of the mammalian cells from the cell culture plates, the mammalian cells were enucleated by density gradient centrifugation using discontinuous Ficoll gradients, high-speed centrifugation. Table 1 summarizes the results of enucleation using a suspension protocol. Enucleation efficiency and cell viability was the highest in both hTERT transformed and primary mesenchymal stem cells (MSCs), as well as in fibroblasts and neutrophils. Table 2 summarizes the results of enucleation using an adherent protocol. Enucleation efficiency was greater than 70% in both mesenchymal stem cells and macrophages. This experiment showed that various types of mammalian cells could undergo enucleation using any of the methods described herein.

TABLE 1
Enucleation efficiency and viability determinations of mammalian
cells using the suspension protocol.

| | | | | Viability | |
|---|---|---|---|---|---|
| | | Enucleation | Recovery | after | Yield per |
| Cell type | | Efficiency | Rate | 24 hours | run |
| MSC cells | AD-MSC | 90%-95% | 60%-90% | 80%-95% | 12-15M |
| | (hTERT) | | | | |
| | UC-MSC | 85%-90% | 60%-80% | 80%-95% | 10-15M |
| | (primary) | | | | |
| | BM-MSC | 80%-90% | 40%-50% | 80%-90% | ~8M |
| | (primary) | | | | |
| NK cells | NKL | 50%-85% | 20%-50% | 50%-75% | ~8M |
| | NK-92 | 70%-90% | 20%-40% | 20%-40% | ~5M |
| Macrophages | RAW | 85%-95% | 40%-70% | 20%-40% | ~15M |
| | 264.7 | | | | |
| Neutrophils | HL-60 | 60%-98% | 20%-40% | 60%-80% | ~15M |
| Fibroblasts | L929 | 70%-90% | 50%-70% | 70%-90% | ~15M |
| | NIH3T3 | 70%-80% | 40%-50% | 70%-80% | ~9M |

Enucleation efficiency = enucleated cells versus total recovered cells;
Recovery rate = recovered cells versus total input cells used for enucleation.
Viability after 24 hours = live cells measured by Trypan blue staining versus total cells;
Yield per run = the number of cytoplasts harvested for each run;
M = million cells
AD-MSC (hTERT) = human hTERT immortalized adipose-derived mesenchymal stem cells;
BM-MSC (primary) = human primary bone marrow-derived mesenchymal stem cells;
NK = natural killer cells.

TABLE 2
Enucleation efficiencies and viability determinations of mammalian
cells using the adherent protocol

| | | | | Viability | |
|---|---|---|---|---|---|
| | | Enucleation | Recovery | after | Yield |
| Cell type | | Efficiency | Rate | 24 hours | per run |
| MSC cells | AD-MSC | 70%-95% | 40%-60% | 80%-95% | 1M |
| | (hTERT) | | | | |
| Macrophages | RAW 264.7 | 85%-95% | 40%-70% | 10%-30% | ~1M |

Enucleation efficiency = enucleated cells versus total recovered cells;
Recovery rate = recovered cells versus total input cells used for enucleation.
Viability after 24 hours = live cells measured by Trypan blue staining versus total cells;
Yield per run = the number of cytoplasts harvested for each run;
M = million cells

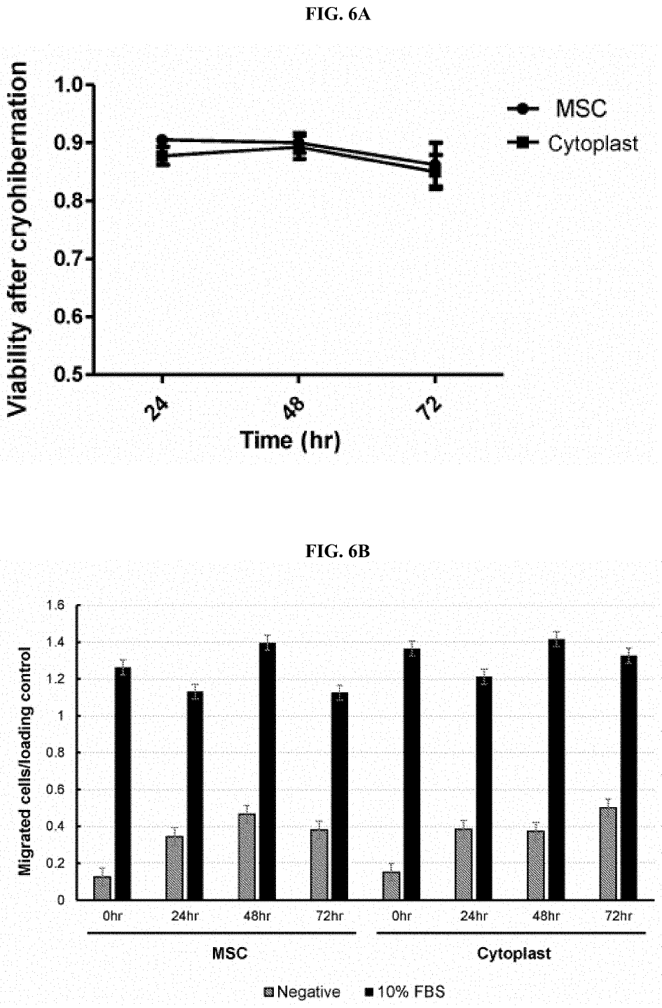

Next, the survival of cytoplasts was determined across 96 hours. Whereas MSC proliferated over-time, cytoplasts did not. Instead, the relative fold change in viable cytoplasts remained fairly constant for 72 hours before declining at 96 hours. Thus, cytoplast survival spanned 3-4 days. As most cell-based therapies are not used immediately, the viability of cytoplasts after cryopreservation was determined. Surprisingly, the viability of cytoplast after cryopreservation was greater than the viability of MSC following cryopreservation. Cytoplasts plated immediately after enucleation and cytoplasts recovered from cryopreservation displayed similar relative cell viability after 24 hours. This experiment showed that cytoplasts survival was not affected by cryopreservation. Additionally, the viability of cytoplasts after cryohibernation was similar to the viability of MSC following cryohibernation (FIG. 6A). Cytoplasts recovered after cryohibernation for various lengths of time were able to undergo induced migration in a Boyden chamber assay similar to MSCs recovered after cryohibernation, (FIG. 6B).

Next, a large-scale production of cells was set up ex vivo, followed by large-capacity density gradient centrifugation and enucleation, which lead to the generation of a therapeutic cytoplast. In one embodiment, the therapeutic cytoplast is loaded with therapeutic cargo (e.g., mRNA, drugs, peptides, etc.) for disease treatment. In another embodiment, the therapeutic cytoplast is prepared for immediate use (e.g., for intravenous injection (IV), intraperitoneal injection (IP), tissue, or in vitro applications) for diagnostic use.

### Example 5

### Cytoplasts Possess Organelles, Interact with the Extracellular Matrix, Perform Cell-Biological Functions, and Deliver Cargo

After determining whether cytoplasts could retain viability after cryopreservation, flow cytometry analysis were performed in order to determine whether the cell surface marker profile of MSC-derived cytoplasts differed from bone-marrow derived MSC. Both MSC-derived cytoplasts and bone-marrow derived MSCs maintained cell surface expression of CD45, CD90, CD44, CD146, and CD166. Cytoplasts attached, reorganized the cytoskeleton, spread on matrix proteins in 2D and 3D culture systems, and formed tunneling nanotubes, which can transfer bioproducts between cells of the same or different origin. Organelle-staining indicated that Golgi, ER, F-actin cytoskeleton, lysosomes, endosomes, microtubules, and mitochondria remain intact in cytoplasts. Furthermore, cytoplasts exhibited homing potential in vitro. Cytoplasts readily migrated on extracellular matrix proteins and migrated directionally towards soluble chemokine gradients (via chemosensing). Notably, cytoplasts transfected exogenously with purified mRNAs produced functional intracellular proteins, which could mimic therapeutic mRNA applications being developed for a variety of clinical uses and disease-states. This also demonstrates that the machineries for mRNA translation and protein synthesis operate normally in cytoplasts in the absence of a nucleus, and thus can be used to produce bioactive molecules with therapeutic value.

Cytoplasts transfected exogenously with purified mRNA encoding known secreted proteins produce functional extracellular proteins in conditioned culture media, indicating that the ER/Golgi and secretory pathways operate normally in cytoplasts in the absence of a nucleus. In addition, treatment of macrophages and endothelial cells with cytoplast-conditioned media containing secreted proteins activated key signal transduction responses in these cells. This provided a proof of concept that cytoplasts could be used as novel vehicles to produce and deliver secreted proteins and biomolecules with therapeutic value. Cytoplasts can be loaded with various cargo including, but not limited to, siRNA, shRNA, mRNA, DNA plasmids, peptides, and chemotherapeutic agents.

### Example 6

### Engineered Cytoplasts Can Express Functional Cell Surface Proteins

Engineered MSCs expressing CXCR4 and engineered MSC-derived cytoplasts expressing CXCR4 express comparable levels of CXCR4, as determined by flow cytometry. To determine whether engineered cytoplasts can express functional cell surface proteins, MSCs and MSC-derived cytoplasts expressing CXCR4 receptors were allowed to migrate towards various concentrations of SDF-1α. MSC-derived cytoplasts engineered to express functional CXCR4 can migrate towards SDF-1α, and cell migration increases with increasing concentrations of SDF-1α. Furthermore, the number of migrating MSC-derived cytoplasts was greater than the number of migrating MSCs expressing CXCR4.

MSC-derived cytoplasts can be engineered to express functional cell adhesion proteins known to mediate cell adhesion to the inflamed vasculature. MSC-derived cytoplasts can be engineered to express cell proteins known to modulate macrophage interactions and phagocytosis of therapeutic cells.

### Example 7

### Engineered Cytoplasts can Function Both In Vitro and In Vivo

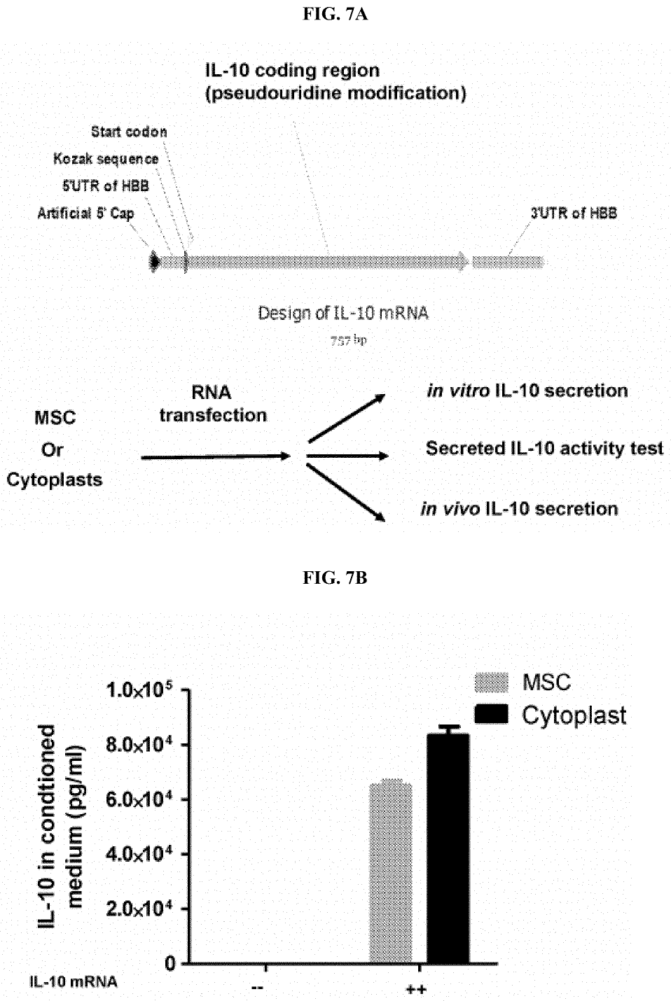

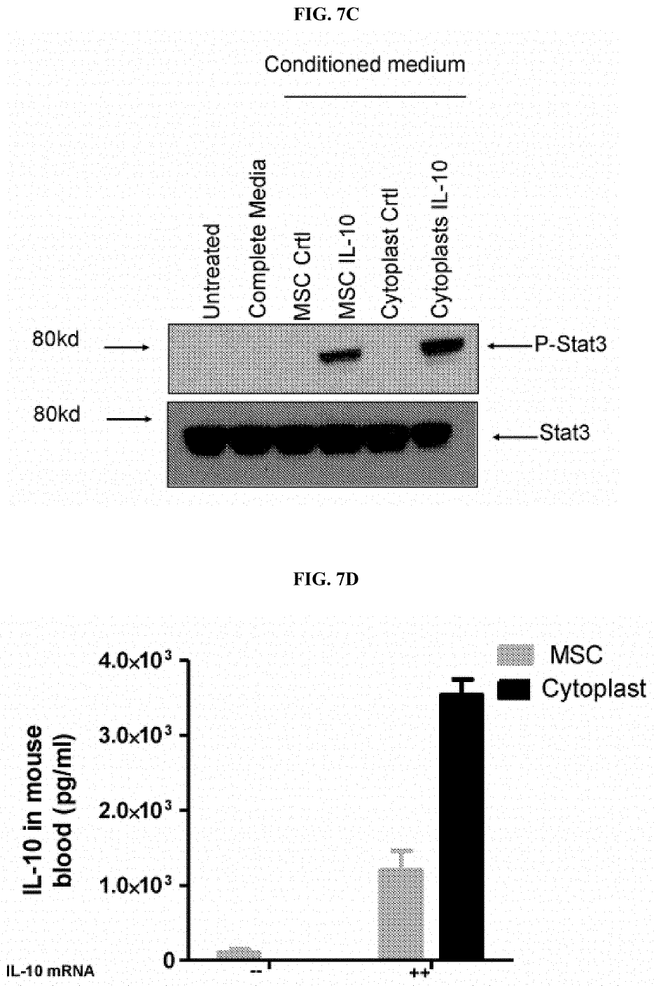

Without wishing to be bound by theory, the examples show that cytoplasts that have been engineered to express a “cargo”, e.g., an exogenous mRNA molecule, can be produced. FIG. 7B and FIG. 7C show that MSC-derived cytoplasts can be engineered to produce and secrete therapeutic levels of a functional anti-inflammatory cytokine interleukin 10 (IL-10) in vitro and in a preclinical mouse model following intravenous injection. FIG. 7B shows that cytoplasts transfected with IL-10 mRNA can secrete high levels of IL-10. To determine whether the secreted IL-10 is active, serum-starved macrophages were incubated with conditioned medium (CM) from untreated MSCs, MSCs expressing IL-10, untreated cytoplasts, and cytoplasts expressing IL-10. Phosphorylated STAT3 was detected in macrophages following incubation with CM from MSCs expressing IL-10 and following incubation with CM from cytoplasts expressing IL-10, whereas no STAT3 activity was detected in macrophages following incubation with CM from untreated MSCs and untreated cytoplasts (FIG. 7C). To determine whether cytoplast-secreted IL-10 can be detected in vivo, C57B1/6 mice were injected retro-orbitally with MSC or MSC-derived cytoplasts expressing IL-10. Two hours post-injection, blood was collected and the levels of IL-10 were determined. Little to no IL-10 was detected in the blood of mice that were injected with untreated MSC (FIG. 7D). As shown in FIG. 7D, higher levels of IL-10 were detected in mice injected with MSC-derived cytoplasts expressing IL-10 as compared to the level in mice injected with untreated MSC.

These data illustrate the potential of genetically engineered cytoplast-based cell therapies to produce and secrete clinically-relevant therapeutic cytokines to treat normal and diseased tissues.

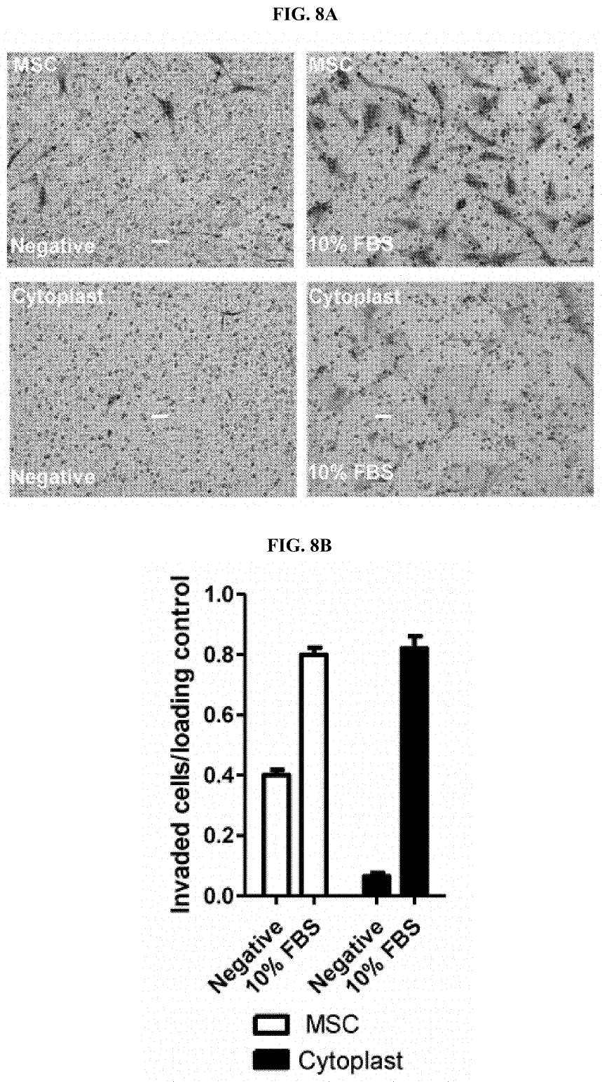

To determine whether MSC-derived cytoplasts can invade through the basement membrane, MSC or MSC-derived cytoplasts were allowed to invade through the basement membrane towards 10% FBS for 24 hours. As shown in FIG. 8A and FIG. 8B, MSC-derived cytoplasts were just was efficient at invading the basement membrane as untreated MSCs in the presence of 10% FBS. Noteworthy, while untreated MSCs were able to invade the basement membrane in the absence of a chemoattractant, MSC-treated cytoplasts were far less able to invade the basement membrane in the absence of a chemoattractant. These data illustrate that MSC-derived cytoplasts can digest and invade through the basement membrane. These data illustrate the innate potential of cytoplast-based cell therapies to penetrate and migrate through complex extracellular matrix barriers to deliver their cargo(s) within tissues.

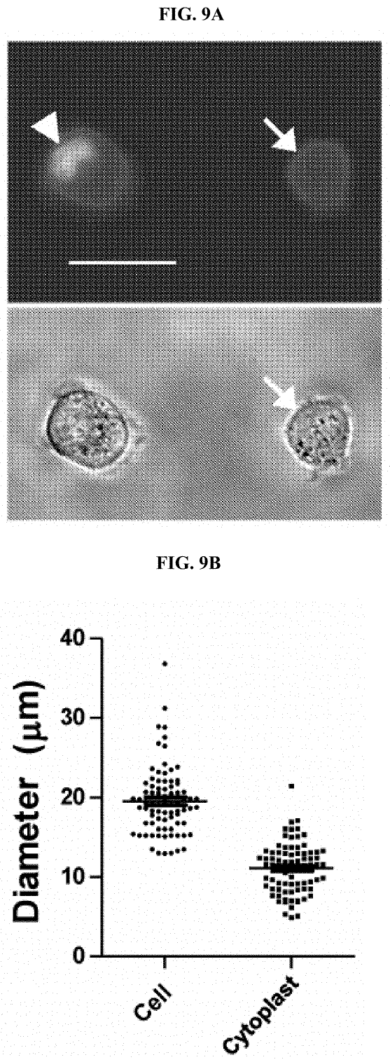

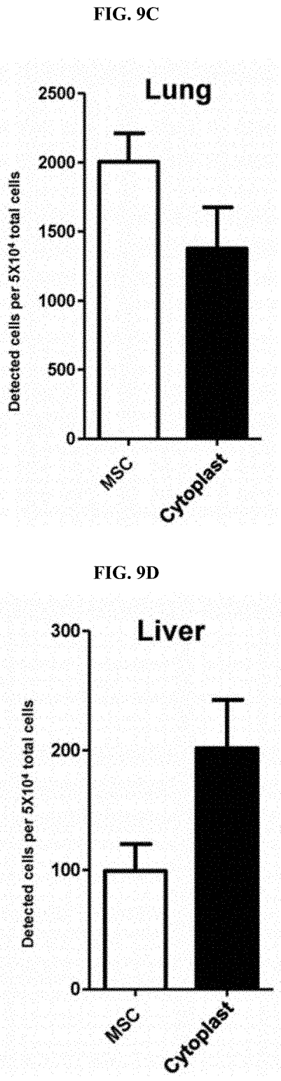

As shown in FIG. 9A and FIG. 9B, MSC-derived cytoplasts have an average diameter of 12 μm, while MSC have an average diameter of 20 μm. To determine the biodistribution of MSC-derived cytoplasts, mice were retro-orbitally injected with MSC or MSC-derived cytoplasts. As shown in FIG. 9C and FIG. 9D, more MSC-derived cytoplasts were detected in the liver than the number of MSC detected in the liver. These data illustrate the potential of cytoplast-based cell therapies to be delivered directly to the circulation to treat a wide range of diseases.

### Example 8

### Exemplary Methods for Generating Cytoplasts

Enucleation of Mesenchymal Stem Cells (MSC)

This protocol was modified from Methods in Cell Biology Volume 14, 1976, Pages 87-93 Chapter 7 Enucleation of Mammalian Cells in Suspension (Michael H. Wigler, Alfred I. Neugut, I. Bernard Weinstein).

Preparation of 50% Ficoll solution: In a glass beaker shielded from light, grams of Ficoll (PM400, GE Healthcare 17-0300-500) were dissolved in an equivalent number of milliliters ultrapure water (Invitrogen 10977-015) by continual magnetic stirring for 24 hours at room temperature. The mixture was then autoclaved for 30 minutes. Once the mixture was cooled, it was stirred again to ensure uniform consistency. The refractive index was measured on a refractometer (Reichert 13940000), and was in the range of 1.4230-1.4290. Aliquots were stored at −20 degrees Celsius.

Preparation of 2× MEM: For each 50 ml quantity, 10 mL 10× MEM (Gibco, 11430-030), 2.94mL exactly Sodium Bicarbonate (7.5%, Gibco, 25080-094), 1 mL 100× Pen-Strep (Gibco 15140-122) and 36 mL ultrapure water (Invitrogen 10977-015) was used. The solution was then filtered through 0.22 um membrane flask (Olympus 25-227) and stored at 4 degrees Celsius.

On the day before enucleation, MSCs were seeded at 2.5 M per 15 cm plate (Olympus 25-203) in 20 mL MSC medium [MEM 1× (Gibco 12561-056); 16.5% premium FBS (Atlanta Biologics S1150); 1% HEPES 1M (Gibco 15630−80); 1% Anti-Anti 100× (Gibco 15240-062); 1% Glutamax 100X (Gibco 35050-061)]. Next, Cytochalasin B (Sigma Aldrich C6762) was added to the 2× MEM (2 μM/mL final concentration).

Preparation of Ficoll gradients: 2× CytoB was added to 50% Ficoll aliquots at 1:1 dilution to make 25% Ficoll stock concentration. Next, 17%, 16%, 15% and 12.5% Ficoll were made by diluting 25% Ficoll with the appropriate volume of 1× MEM buffer (2× MEM containing Cytochalasin B added to ultrapure water at 1:1 dilution). The dilutions were equilibrated in a CO₂ incubator for at least 1 hour covered with loose cap. The Ficoll gradients were then poured into 13.2mL ultra-clear tubes (Beckman, 344059), and incubated overnight (6-18 hours) in the CO₂ incubator.

On the day of enucleation, 12-25M MSC (ideally 20M) were collected into each tube for enucleation. Media was aspirated, and the cells washed once with phosphate buffered saline (PBS) (GIBCO 14190-144). Five mL of TrypLE-Select (Gibco, 12563011) was added to each plate, and incubated up to 5 minutes. When 90% of the cells were detached, 5 mL full MSC media was added, and the cells were collected into 50 ml tubes (3-4 plates/tube). The tubes were then centrifuged at 1, 200 rpm for 5 minutes. The pellet was resuspended in 10 mL PBS. Cells were counted, pelleted, and re-suspended with 12.5% Ficoll. Next, the cell-Ficoll mixture was dropwise passed through a 40 um cell strainer (Falcon 352340) into a new 50 mL tube. Using a syringe, 3.2 mL of cell suspension was slowly loaded onto the pre-made gradients. One mL of 1× MEM buffer was added at the final (top) layer with syringe. The tubes were then loaded into rotor buckets, balanced, and run in the ultracentrifuge (Beckman, L8M) for 60 minutes, 26,000 rpm, 31° C., Accel 7, Deccel 7. At the end of the centrifugation, there were three layers: one near the top of the 12.5% (cytoplasts and debris), one near the 12.5/15% interface (cytoplasts), and a pellet at the bottom of the 25% (karyoplasts). The layers above 15% Ficoll solution were collected into 15 ml conical tubes. The collected layers are then diluted with more than 4 volumes warm serum-free MSC medium (i.e. 3 mL of Ficoll and filled with up to 15 mL media). After gently mixing, the mixture was pelleted for 10 minutes at 1,200 rpm. Following three washes with warm serum-free MSC medium, the cells were resuspended in media according to the experimental protocol, e.g., transfection media vs. migration media vs. serum free media vs. full media. Efficiency of enucleation was determined in a 12-well plate by adding full MSC media with 1:2000 dilution Vybrant® Dyecycle™ Green (Molecular Probes V35004) or 1:5000 dilution Hoechst 33342. A small volume of each layer was added to each well and allowed to attach/stain for 10 minutes in the incubator. The percentage of negative cytoplasts per population was determined by epifluorescent microscopy.

### Cytoplast MRNA Transfection

1 M cytoplasts were suspended with warm 1 ml amino acid-free α-MEM full medium (ThermoFisher 12561056; 16.5% Premium fetal bovine serum (FBS), 1% Glutamax (Gibco 35050061), 1% HEPES (Gibco 15630080)). 1 μg mRNA was diluted with warm opti-MEM and mixed with pipet at least 20 times. 4 μl lipofectamine-3000 (ThermoFisher L300015) was added to 46 μl warm opti-MEM (ThermoFisher 31985062) and mixed with pipet for at least 20 times. The ratio of mRNA and lipofectamine-3000 was 1:4 (w/v). The mRNA and lipofectamine-3000 dilutions were mixed with pipet for at least 20 times and incubated at room temperature for 15 minutes. The mRNA and lipofectamine-3000 mixture was added to the cytoplast suspension, mixed well and incubated at 37° C. for 30 minutes. The suspension was shaken every 5 minutes to prevent cell clumping. After incubation, the cells were centrifuged, and re-suspended in normal α-MEM full medium (16.5% Premium FBS, 1% Antibiotic-Antimycotic, 1% Glutamax, 1% HEPES) or PBS.

### Cytoplast SiRNA Transfection

1 M cytoplasts were suspended with warm 1 ml A/A free α-MEM full medium (16.5% Premium FBS, 1% Glutamax, 1% HEPES). Two μl siRNA was diluted with warm opti-MEM and mixed with pipet at least 20 times. Eight μl lipofectamine-3000 was diluted with 92 μl warm opti-MEM and mixed with pipet at least 20 times. The ratio of siRNA and lipofectamine-3000 was 1:4 (v/v). The siRNA and lipofectamine-3000 dilutions were mixed with pipet at least 20 times and incubated at room temperature for 15 minutes. The siRNA and lipofectamine-3000 mixture was added to the cytoplast suspension, mixed well and incubated at 37° C. for 20 minutes. The suspension was shaken every 5 minutes to prevent cell clumping. After a 20 minute incubation, the cells were centrifuged, and re-suspended with normal α-MEM full medium (16.5% Premium FBS, 1% Antibiotic-Antimycotic, 1% Glutamax, 1% HEPES).

### Generation of Oncolytic Virus Infected Cytoplasts

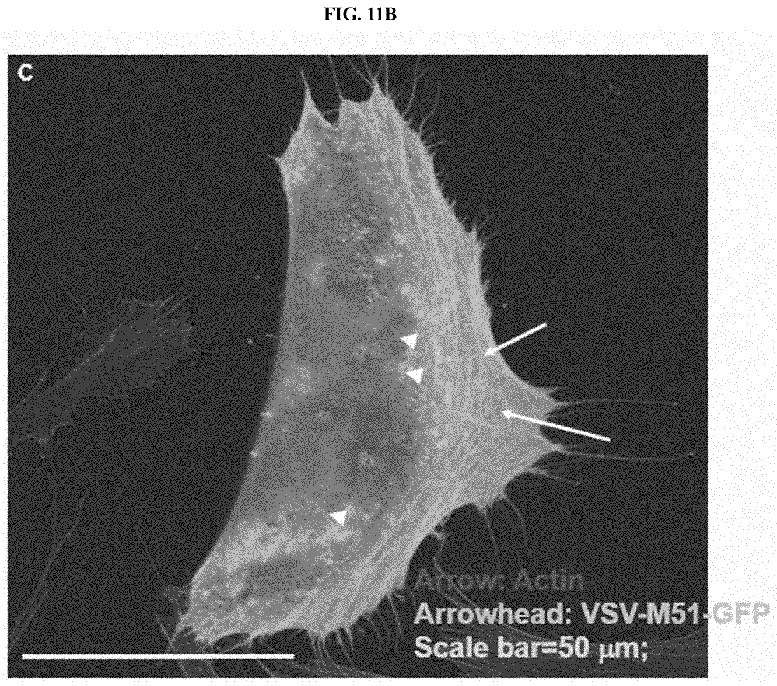

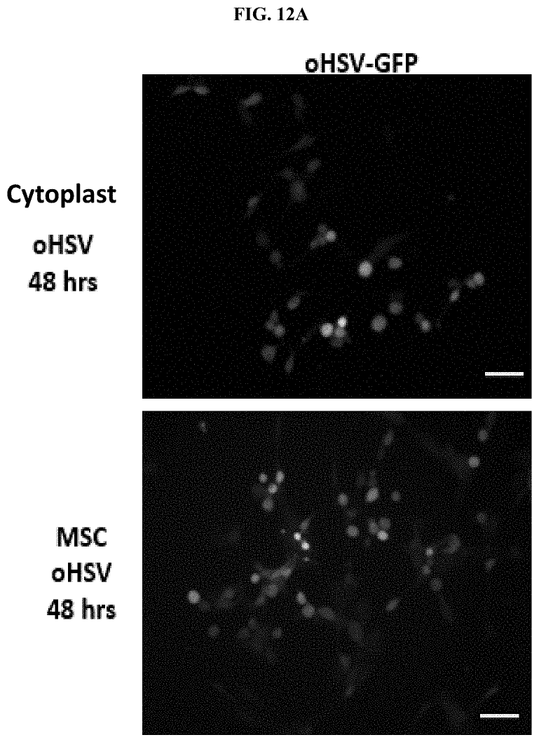

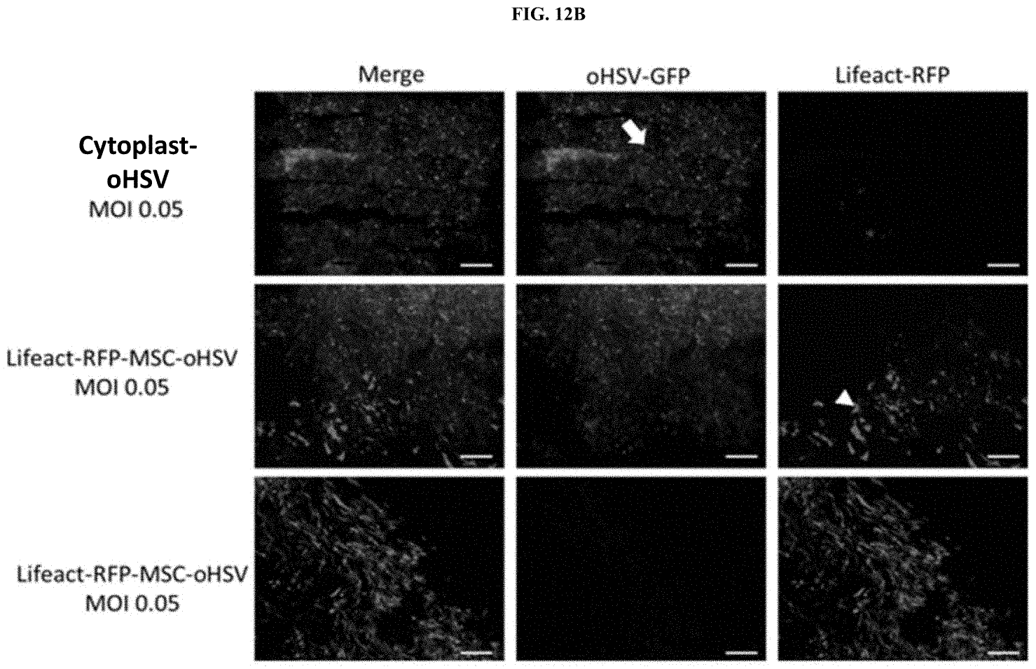

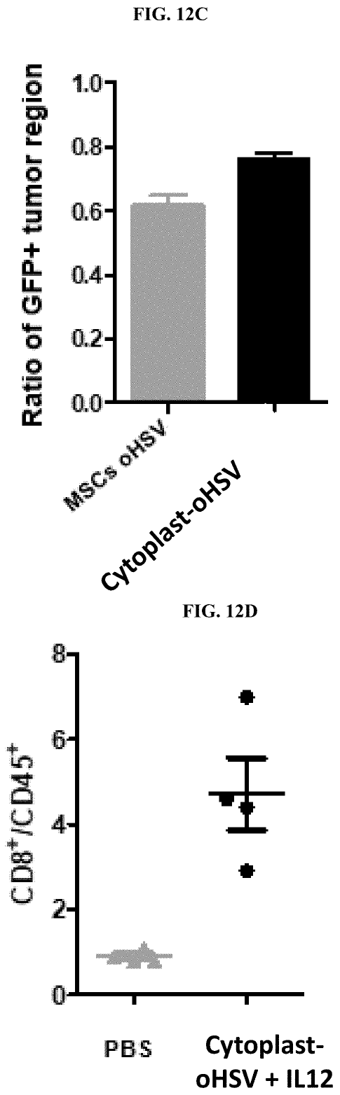

One day before enucleation (usually 18 hrs before enucleation), 2.5*10\hat{ }6 hTERT-MSCs were seeded on a 15-cm dish. Roughly two hours after seeding, the cells were washed once with PBS. Cells were then infected with oHSV-GFP (Imams OV3001) at different MOIs (0.05 or 0.5 for example) with 8 mL serum free opti-MEM. Next, cells were incubated at 37° C. for 2 hours with occasionally shaking. The virus inoculum was then discarded. 20 mL pre-warmed full culture medium (a-MEM, 16.5% Premium FBS, 1% Antibiotic-Antimycotic, 1% Glutamax, 1% HEPES) was added to each well. The cells were incubated at 37° C. until enucleation. FIG. 11 illustrates fluorescent images of introducing polypeptide (VSV-GFP) directly into the parent or reference cell (cell without a nucleus) and into the enucleated cell described herein. FIG. 12 illustrates infecting MSCs with oncolytic Herpes Simplex Virus (oHSV) encoding GFP antigen. FIG. 12C illustrates increased delivery of the cargo (e.g. GFP reporter) to the target cancer cell by the enucleated MSCs. FIG. 12D illustrates increased recruitment of immune cells (e.g., CD8+ effector T cells) to the target cancer cell contacted by the enucleated MSCs described herein.

### Lentivirus Overexpressing Functional Proteins in Cytoplasts

Target cells were plated in one well of 6-well plate at density of 1-2×10⁵ cells/well, or 10 cm plate with 0.5-1 M MSCs. The next day, the concentrated recombinant lentivirus was thawed in a 37° C. water bath and removed from the bath immediately once thawed. The cells were then washed with PBS 3 times. 200 μL serum free medium or 2 mL serum free medium (1:1250 SureENTRY) was added. The target cells were infected in a 6-well plate with MOI 10:1. The next day, the viral supernatant was removed and the appropriate complete growth medium was added to the cells. After 72 hours incubation, the cells were subcultured into 2×100 mm dishes. The appropriate amount of selection drug (i.e. puromycin) was added for stable cell-line generation. 10-15 days after selection, clones were picked for expansion and were screened for positive ones. The selected positive clones were expanded for enucleation. Engineered cytoplasts were prepared as outlined above. The target protein expression on cytoplasts was determined by ordinary biochemical methods or functional assays, e.g., fluorescent activated cell sorting (FACS), western blot, or Boyden chamber assay.

Peptide Loading into Cytoplasts

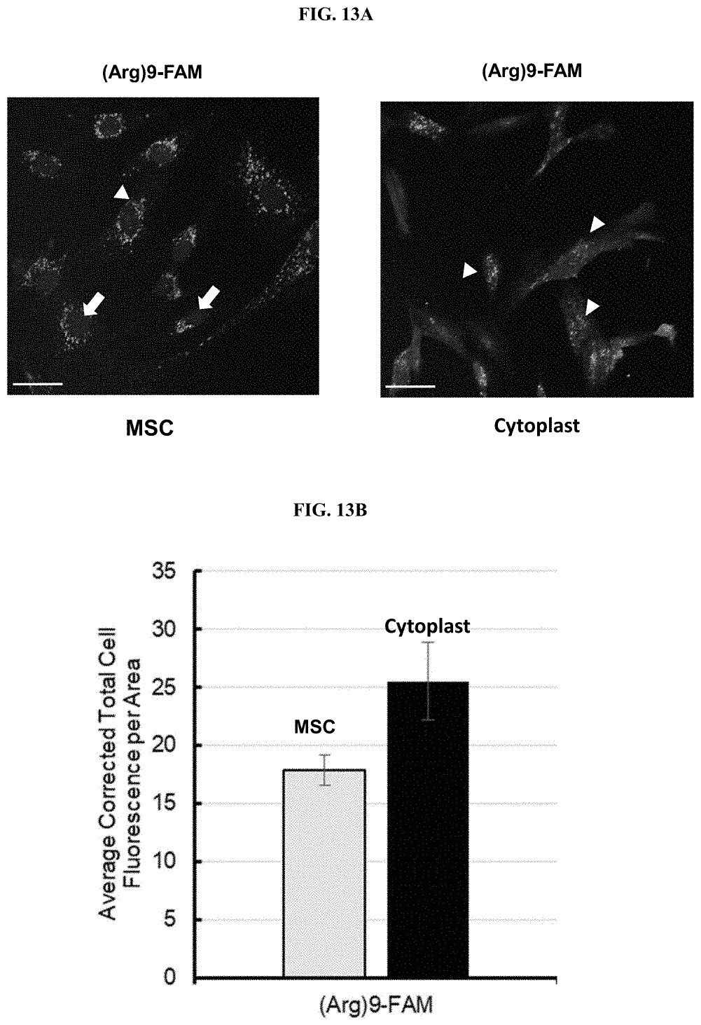

1×10⁵/ml per well were plated onto a 4-chamber glass slide (LabTek II 4-chamber glass slide, 155383) in full MSC media [MEM 1× (Gibco 12561-056); 16.5% premium FBS (Atlanta Biologics S1150); 1% HEPES 1M (Gibco 15630-80); 1% Anti-Anti 100× (Gibco 15240-062); 1% Glutamax 100× (Gibco 35050-061)]. Cells were allowed to attach for at least 1 hour or overnight. Cells were then rinsed with PBS (Gibco 14190-144). Arg9(FAM) (SEQ ID NO: 1154) (10 mM, Anaspec, AS-61207) was diluted in full media to a total concentration of 1:100 (100 uM). Cytoplasts were then incubated for 1 to 2 hours, and rinsed 3 times with PBS. Hoechst 33342 (Invitrogen) was added at a 1:5000 dilution in full media for at least 10 minutes. Cells were then washed with PBS and imaged by epifluorescent microscopy. FIG. 13 illustrates the increased peptide uptake or loading of a polypeptide of interest when co-incubated with the Arg9.

### Example 9

### Cytoplasts Show Better Biodistribution In Vivo

MSCs were cultured in 3D-hanging drops (3D MSCs) then enucleated to generate 3D cytoplasts. The 3D culture protocol of MSC by hanging drops is modified from Curr Protoc Stem Cell Biol. 2014 Feb. 6; 28: Unit-2B.6.(Thomas J. Bartoshl and Joni H. Ylostalo).

Healthy MSCs were harvested from 2D-cultured plates by Trypsin and resuspended in fresh α-MEM (ThermoFisher 12561056) full medium (16.5% Premium FBS, 1% Antibiotic-Antimycotic, 1% Glutamax, 1% HEPES) at 1.43 million cells/ml. The lid of a 15 cm plate was opened completely and 20 ml PBS was added to the plate. A multichannel pipette was used to make droplets on the lid of the plate at 35 μl per droplet (approx. 50,000 cells/droplet). About 100-120 droplets were placed on each lid. The lid was closed and the plate was placed back into the incubator. Droplets were cultured for 2 days, then harvested by cell lifter and collected into 15 ml tubes (approx. 300 droplets per tube). The tubes were centrifuged for 5 minutes at 1,200 rpm. The supernatant was removed and the tubes were washed twice with PBS. All P BS was then removed and 7.5 ml of freshly thawed 0.25% Trypsin-EDTA (ThermoFisher 25200114) was added to each tube. The tubes were incubated in a water bath for 4 minutes. The droplets were gently pipetted with 1 ml pipettes with low-retention tips about 10-20 times and incubated in the water bath for another 4 minutes. The droplets were again gently pipetted with 1 ml pipettes with low-retention tips about 10-20 times until most of the droplets were dissociated. 7.5 ml of full serum medium (GlutaMAX Supplement (Gibco 35050061); Fetal Bovine Serum-Premium Select (Atlanta Biologicals S11550); HEPES (1 M) (Gibco 15630080); antibiotic-Antimycotic (100×) (Gibco 15240062)) was added to each tube and the tubes were centrifuged for 10 minutes at 1,200 rpm. The dissociated cells were washed with 10 ml of full serum medium and the cells were resuspended with 5m1 full serum medium. The cells were passed through a 70 μm cell filter and then the filter was washed with 5 ml full serum medium. The cells were counted and resuspended with pre-treated 12.5% Ficoll at more than 10M/ml. 30-40M cells were used for each enucleation tube. Subsequently, the protocol for enucleation described above was followed.

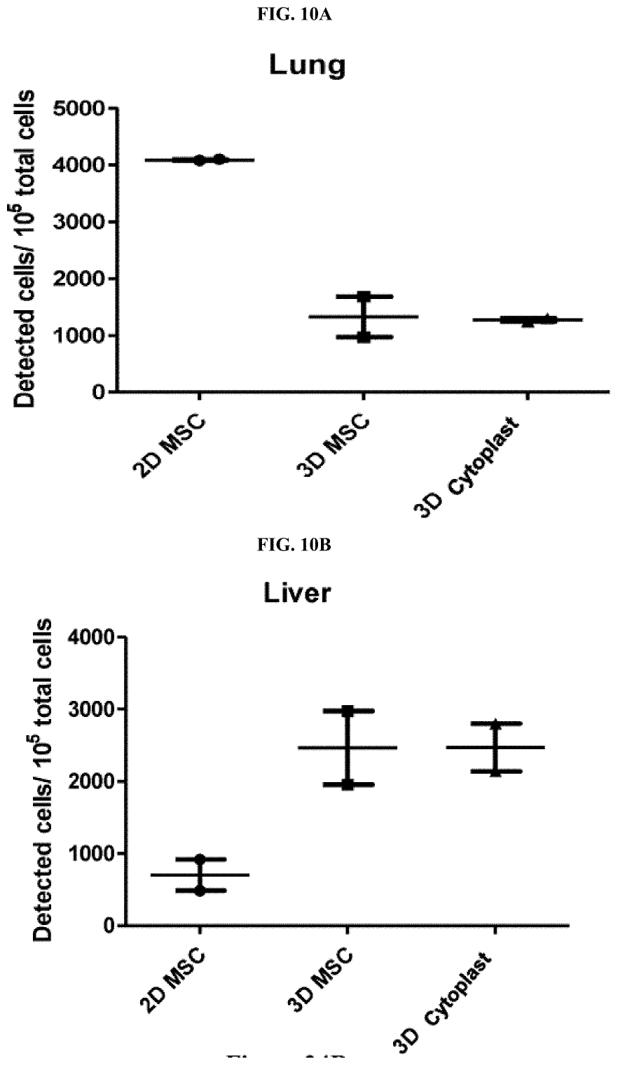

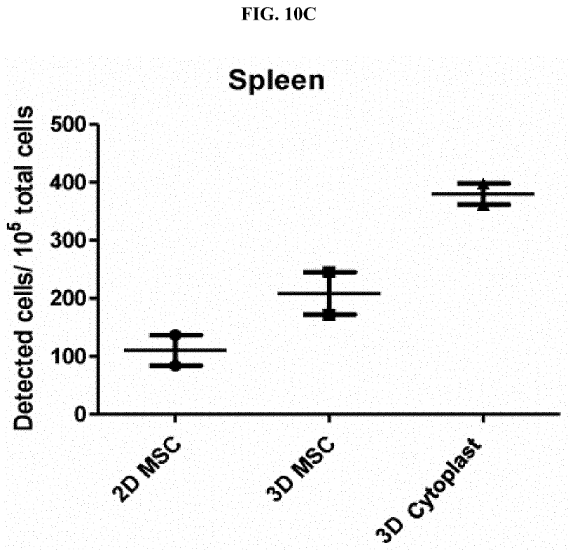

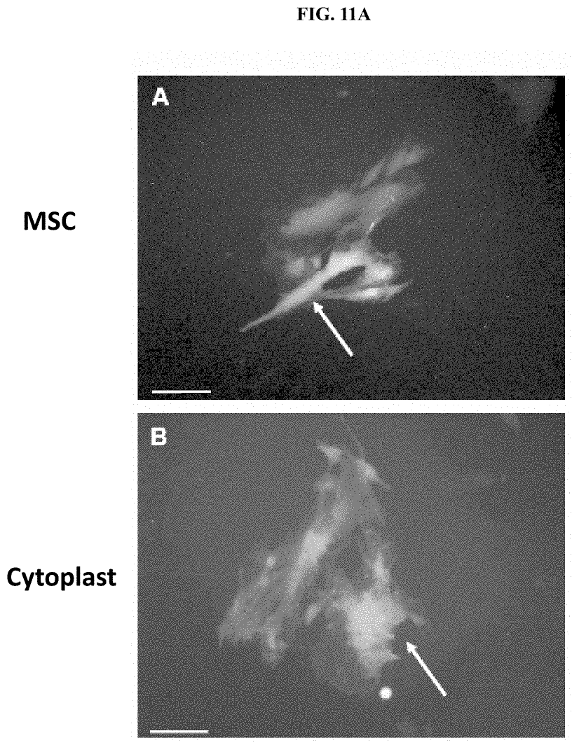

DiD labeled normal 2D-cultured MSCs (2D MSC), 3D MSCs or 3D cytoplasts were retro-orbitally injected into BalB/C mice respectively. Indicated tissues were harvested 24 hours after injection and DiD labeled cells analyzed by FACS. FIG. 10A-10C show the successful generation of 3D-derived cytoplasts from 3D-cultured MSCs and also shows the 3D-derived cytoplasts have less lung trapping and better biodistribution to peripheral organs than 2D-cultured cells after injection into the circulation. This is expected to greatly improve their therapeutic ability to locate and deliver cargo to tissues.

### Example 10

### Methods of Treating a Disease Caused by An Infection

A patient infected with SARS-CoV-2 begins experiencing symptoms of Coronavirus disease 2019 (COVID-19). Respiratory symptoms of COVID-19 include shortness of breath and/or difficulty breathing.

The patient is administered a pharmaceutical formulation containing the cytoplasts described herein expressing an agonist of interleukin 10 (IL-10), or a portion thereof that is sufficient to treat the respiratory symptoms of the COVID-19 in the subject. In this example, the cytoplasts also expresses homing receptors that target the lymph tissue to enable efficient homing of the cytoplasts to the lymphatic system. The cytoplasts also expresses immune-evading moieties, such as a “don't eat me” signally peptide to ensure the cytoplasts are not cleared from the subject before reaching the lymphatic system. Following administration, the respiratory symptoms of the subject are reduced following administration.

### Example 11

### Generation of Oncolytic Virus Infected Cytoplasts

One day before enucleation (usually 18 hrs before enucleation), 2.5*10\hat{ }6 hTERT-MSCs were seeded on a 15-cm dish. Roughly two hours after seeding, the cells were washed once with PBS. Cells were then infected with oHSV-GFP (Imams OV3001) at different MOIs (0.05 or 0.5 for example) with 8 mL serum free opti-MEM. Next, cells were incubated at 37° C. for 2 hours with occasionally shaking. The virus inoculum was then discarded. 20 mL pre-warmed full culture medium (α-MEM, 16.5% Premium FBS, 1% Antibiotic-Antimycotic, 1% Glutamax, 1% HEPES) was added to each well. The cells were incubated at 37° C. until enucleation. FIG. 11A-11B illustrates fluorescent images of introducing polypeptide (VSV-GFP) directly into the parent or reference cell (cell without a nucleus) and into the enucleated cell described herein. Epifluorescent microscopy images of nucleated parental MSCs (top) and MSC-derived cell without nucleus (bottom) infected with VSV-GFP (arrow) at MOI 0.05 at 12 hrs after infection illustrated the introduction of a reporter peptide, GFP, into the MSC. The GFP antigen was clearly and robustly expressed by MSCs without nuclei indicating viral replication and antigen production in enucleated cells. Scale bar=50 μm. FIG. 11B illustrates high magnification epifluorescent image of an MSC-derived cell without nucleus infected with VSV-GFP (arrowheads) at MOI 0.1 at 12 hours after infection. The enucleated cell was also stained for F-actin filaments using rhodamine phalloidin (arrows) and the nuclear stain DAPI to illustrate the lack of the nucleus. FIG. 11 illustrates that cytoplasts can be engineered and transfected with oncolytic virus to express exogenous peptide such as antigenic peptide. FIG. 11 also illustrates that cytoplasts can be infected by virus for viral-trapping purpose.

### Example 12

### Recruitment and Activation of Immune Response to Target Cells Contacted by Cytoplasts

One day before enucleation (usually 18 hrs before enucleation), 2.5*10\hat{ }6 hTERT-MSCs were seeded on a 15-cm dish. Roughly two hours after seeding, the cells were washed once with PBS. Cells were then infected with oHSV-GFP (Imanis OV3001) at different MOIs (0.05 or 0.5 for example) with 8 mL serum free opti-MEM. Next, cells were incubated at 37° C. for 2 hours with occasionally shaking. The virus inoculum was then discarded. 20 mL pre-warmed full culture medium (α-MEM, 16.5% Premium FBS, 1% Antibiotic-Antimycotic, 1% Glutamax, 1% HEPES) was added to each well. The cells were incubated at 37° C. until enucleation. FIG. 12A-12BD illustrates infecting MSCs with oncolytic Herpes Simplex Virus (oHSV) encoding GFP antigen. Epifluorescent microscopy images of MSC and MSC without nucleus illustrate infection with oHSV encoding GFP antigen at MOI 0.05 at 48 hrs after infection. MSCs without nuclei were generated from MSCs 18 hrs after inoculation with oHSV-GFP. Scale bar=50 μm. FIG. 12B illustrates MSCs or MSCs without nuclei expressing lifeact-RFP after infected with 0.05 MOI of the oncolytic herpes simplex virus encoding GFP (oHSV-GFP) then injected into established U87 glioblastoma tumors growing in Nude mice. Images were taken 7 days after the injection. Both MSCs and MSCs without nuclei delivered oHSV to tumor cells as indicated by the strong GFP signal. It was noted that very few MSCs without nuclei could be detected in the tumor after 7 days, whereas a large number of MSCs were present in the center (injection site) and at the outer edge of the growing tumor. FIG. 12C is a bar graph showing percentage of GFP-covered tumor area, which represents the portion of tumor cells infected by MSCs or MSCs without nuclei carrying the oHSV-GFP virus. FIG. 12D is a graph showing the increased ratio of CD8+ effector T cells present in established glioblastoma tumors treated with combination of IL-12 (adjuvant) engineered MSCs without nuclei and oHSV engineered MSCs without nuclei compared to PBS injected controls. FIG. 12 illustrates that the cytoplasts described herein can induce sufficient immune response by recruiting immune cells to the site of the engineered cytoplasts. In such scenario, the cytoplasts and any cargo encapsulated by the cytoplasts (e.g., the virus that is trapped inside the cytoplasts) would be subjected destruction by the recruited immune response.

### Example 13

### Peptide Loading into Cytoplasts

1×10⁵/ml per well were plated onto a 4-chamber glass slide (LabTek II 4-chamber glass slide, 155383) in full MSC media [MEM 1× (Gibco 12561-056); 16.5% premium FBS (Atlanta Biologics S1150); 1% HEPES 1M (Gibco 15630-80); 1% Anti-Anti 100× (Gibco 15240-062); 1% Glutamax 100× (Gibco 35050-061)]. Cells were allowed to attach for at least 1 hour or overnight. Cells were then rinsed with PBS (Gibco 14190-144). Arg9(FAM) (SEQ ID NO: 1154) (10 mM, Anaspec, AS-61207) was diluted in full media to a total concentration of 1:100 (100 uM). Cytoplasts were then incubated for 1 to 2 hours, and rinsed 3 times with PBS. Hoechst 33342 (Invitrogen) was added at a 1:5000 dilution in full media for at least 10 minutes. Cells were then washed with PBS and imaged by epifluorescent microscopy. FIG. 13A-13B illustrates increased peptide uptake or loading of a polypeptide of interest when co-incubated with the Arg9. As shown in FIG. 13A, MSCs (left) and enucleated MSCs (right) illustrate MSCs incubated with 100 μM of the cell-permeable antigen peptide (Arg)9-FAM (6-Carboxyfluorescein, FAM-Arg-Arg-Arg-Arg-Arg-Arg-Arg-Arg-Arg-OH). Scale bar=50 μm. Arrows indicate Hoechst stained nuclei, arrowheads indicate positive (Arg)9-FAM. FIG. 13B illustrates bar graphs represents relative fluorescence intensity measured in ImageJ. Corrected Total Cell Fluorescence=Integrated Density−(Area of selected cell X Mean fluorescence of background readings). Mean±SEM; n=10. Overall, FIG. 13 illustrates that the cytoplasts described herein (e.g., the MSCs without nuclei) can be directly loaded with a polypeptide of interest. For example, an antigen can be introduced into the cytoplasts by co-incubation of the antigen and the Arg9(FAM) with the cytoplasts. These cytoplasts can then function as the vaccine described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. An enucleated cell, comprising:
(a) an oncolytic virus comprising a viral genome that encodes a cancer antigen; and
(b) one or more organelles for release of the oncolytic virus from the enucleated cell.

2. The enucleated cell of claim 1, wherein the caner antigen comprises a tumor antigen.

3. The enucleated cell of claim 1, wherein the oncolytic virus comprises a herpes simplex virus, an adenovirus, an adeno-associated virus, a human papillomavirus, a hepatitis virus, a vaccinia virus, a vesicular stomatitis virus, a poliovirus, a reovirus, a senecavirus, an echo virus, or a Semliki Forest virus.

4. The enucleated cell of claim 3, wherein the oncolytic virus is a herpes simplex virus.

5. The enucleated cell of claim 1, further comprising an immune-modulator.

6. The enucleated cell of claim 5, wherein the immune-modulator comprises a cytokine, a major histocompatibility complex (MHC) class II epitope peptide, a damage-associated molecular pattern (DAMP) molecule, a pathogen-associated molecular pattern (PAMP) molecule, a Toll-like receptor (TLR) agonist, a stimulator of interferon genes (STING) agonist, a retinoic acid-inducible gene I (RIG-I) agonist, or an adjuvant.

7. The enucleated cell of claim 1, wherein the enucleated cell is an enucleated mesenchymal stromal cell or an enucleated mesenchymal stem cell.

8. The enucleated cell of claim 1, wherein the one or more intracellular organelles comprises a endoplasmic reticulum or a Golgi apparatus.

9. The enucleated cell of claim 1, further comprising a homing receptor specific to a cancer cell.

10. The enucleated cell of claim 1, wherein the enucleated cell is not a red blood cell or a platelet.

11. A method of treating a cancer in a subject, the method comprising:
administering an enucleated cell to the subject, wherein the enucleated cell comprises (i) an oncolytic virus, (ii) a cancer antigen, and (iii) one or more organelles, wherein the one or more organelles releases the oncolytic virus, the cancer antigen, or a combination thereof from the enucleated cell in vivo, thereby delivering the oncolytic virus to a cancer cell of the subject to induce death of the cancer cell.

12. The method of claim 11, wherein the caner antigen comprises a tumor antigen.

13. The method of claim 11, wherein the oncolytic virus comprises a viral genome that encodes the cancer antigen.

14. The method of claim 11, wherein the cancer is lung cancer or cancer in lung tissue.

15. The method of claim 11, wherein the oncolytic virus comprises a herpes simplex virus, an adenovirus, an adeno-associated virus, a human papillomavirus, a hepatitis virus, a vaccinia virus, a vesicular stomatitis virus, a poliovirus, a reovirus, a senecavirus, an echo virus, or a Semliki Forest virus.

16. The method of claim 15, wherein the oncolytic virus is a herpes simplex virus.

17. The method of claim 11, wherein the one or more intracellular organelles comprises a endoplasmic reticulum or a Golgi apparatus.

18. The method of claim 11, wherein the enucleated cell further comprises a homing receptor, wherein the homing receptor guides the enucleated cell to the cancer cell of the subject in vivo.

19. The method of claim 11, wherein the enucleated cell is not a red blood cell or a platelet.

20. The method of claim 11, further comprising administering an immune-modulator.
